(19) United States
(12) Patent Application Publication
(10) Pub. No.: US 2012/0043208 A1
(43) Pub. Date: Feb. 23, 2012

(54) APPARATUS AND METHODS FOR HIGH THROUGHPUT BIOMOLECULE SEPARATION AND ANALYSIS

(75) Inventors: Jian Jin, Berkeley, CA (US); Mark D. Biggin, Berkeley, CA (US); Robert A. Nordmeyer, San Leandro, CA (US); Ming Dong, El Cerrito, CA (US); Earl W. Cornell, Antioch, CA (US); Megan Choi, El Cerrito, CA (US); Halina Ewa Witkowski, Walnut Creek, CA (US); Bong-Gyoon Han, Castro Valley, CA (US); Robert M. Glaeser, Berkeley, CA (US)

(73) Assignee: The Regents of the University of California, Oakland, CA (US)

(21) Appl. No.: 13/176,704

(22) Filed: Jul. 5, 2011

Related U.S. Application Data
(63) Continuation of application No. PCT/US2010/020167, filed on Jan. 5, 2010.
(60) Provisional application No. 61/142,595, filed on Jan. 5, 2009.
(60) Provisional application No. 61/160,276, filed on Mar. 13, 2009.

(51) Int. Cl. G01N 33/559; C25B 7/00; G01N 27/447

(52) U.S. Cl. 204/452; 204/605; 204/455

(57) ABSTRACT

A multi-channel gel electrophoresis apparatus for efficiently collecting molecules isolated by gel electrophoresis so they can be further analyzed, identified, or used as reagents or medications. The apparatus using a novel “tagless” strategy that combines multi-dimensional separation of endogenous complexes with mass spectrometric monitoring of their composition. In this procedure, putative protein complexes are identified based on the co-migration of collections of polypeptides through multiple orthogonal separation steps. A majority of E. coli proteins are shown to remain in stable complexes during fractionation of a crude extract through three chromatographic steps.

### CROSS-REFERENCE TO RELATED APPLICATIONS

This application claims priority from U.S. Provisional Patent Application, 61/142,595, filed on Jan. 5, 2009, and U.S. Provisional Patent Application, 61/160,276, filed on Mar. 13, 2009, and International Application No. PCT/US2010/020167 filed on Jan. 5, 2009, all of which are hereby incorporated by reference in their entirety.

### STATEMENT OF GOVERNMENTAL SUPPORT

This invention was made during work supported under Contract No. DE-AC02-05CH11231 awarded by the U.S. Department of Energy. The government has certain rights in this invention.

### BACKGROUND OF THE INVENTION

1. Field of the Invention

The present invention relates to field of biomolecule separation and purification, assays that identify protein complexes from whole cells and high-throughput devices for carrying out such methods.

2. Related Art

Most proteomics analyses employ a basic approach that combines a multi-dimensional separation scheme with a protein identification technique involving mass spectrometry. This reflects the simple fact that complex mixture of proteins must be well separated and simplified prior to identification, and only mass spectrometry has the adequate sensitivity and throughput for such analysis. Polyacrylamide gel electrophoresis (PAGE) is one of the most important and effective protein separation technique and has been incorporated in various multi-dimensional schemes. For example, traditional two-dimensional gel electrophoresis (2DE) has been established for many years as a robust method for separation of complex protein lysates, especially the soluble proteins, obtained from cells. The power of 2DE stems from the fact that both the isoelectrical-focusing (IEF) and the SDS gel electrophoresis are highly resolving, dynamic, and orthogonal chromatographic techniques. The two-dimensional blue native/SDS gel electrophoresis (2D BN/SDS-PAGE) has been the “method of choice” for intact membrane proteins and protein complexes.

Similarly, gel electrophoresis is a main tool for protein purification involving “tagging” strategies. However, a major limitation of PAGE, compared with some other liquid chromatography techniques, is that it is not readily interfaced with mass spectrometry because it is inconvenient to get separated protein bands out of the gel for further processing. To harvest separated proteins, one must cut them out or elute. Large scale “gel-cutting” has been used in proteomics and protein interaction projects and it is an effective but labor intensive approach. A gel-cutting robot, for instance, the “Ettan Spot Picker” from GE Healthcare's Life Sciences (Piscataway, N.J.) exists but its accessibility is generally limited. For elution, there are at least two types of elution devices on the market—“Model 491 Prep Cell” also described in U.S. Pat. No. 4,877,510 hereby incorporated by reference and “Whole Gel Eluter”—both from Bio-Rad Co. (Hercules, Calif.). They are not widely used, partially because they can process only one or two samples a time; sample loss is significant and it is difficult to operate. “The Mini Prep Cell” or “Model 491 Prep Cell” can be used to purify specific proteins or nucleic acids from complex mixtures by continuous-elution electrophoresis. Separated protein bands migrate off the bottom of the gel column and a complicated but not vary effective elution chamber traps and collects the bands. Other than its poor efficiency, it is not suitable for eluting and collecting bands in a dynamic range of protein mass, nor parallel multi-channel processing. Another device, “Whole Gel Eluter” allows simultaneous elution of multiple bands of proteins already separated by a slab gel, and the elution is in the transverse direction, through the thickness of the gel. The collection and recovery of eluted protein bands are difficult and ineffective. In addition, the experiment set-up procedure of this device is tedious and technically challenge; it can process only one sample per run.

It is essential to find an effective way to collect eluted bands. Important issues regarding fraction collection include interruption of electrophoresis, continuity of collection, loss of sample, loss of resolution and dilution, etc. Previously several approaches, although primarily designed for capillary electrophoresis (CE), had been investigated. Three approaches of particular interest are: (1) sweep liquid: liquid was swept through a standard liquid chromatographic detector, resulting in continuous and uninterrupted collection but sample dilution; (2) on-line frit: a frit structure was attached to the outlet end of the capillary to isolate electrical conductivity from the elution buffer flow (Huang, X.; Zare, R. N. Anal. Chem. 1990, 62, 443-446); and (3) coaxial sheath flow: a sheath flow was built around the capillary to confine the sample flow but also provide the electrical connection to ground electrode (Muller, O.; Foret, F; Karger, B. Anal. Chem. 1995, 67, 2974-2980). Each of these methods has its own advantages and disadvantages.

In recognition of this need in proteomics, the US Department of Energy's program, has established as a major goal the development of very high throughput methods to characterize the structures and functions of protein complexes in microbes relevant to its mission. In the Protein Complex Analysis Project (PCAP), our high throughput pipelines include methods that employ various “tagging” strategies and 2D BN/SDS-PAGE approach, and a common step in these methods is the use of SDS-gels for protein separation and purification, followed by protein identification by mass spectrometry.

It would be a significant advance in the field of proteomics if a high throughput gel-eluting tool were developed to interface with mass spectrometry. Genome sequencing projects have identified the complete set of proteins for many organisms. To take advantage of this information, the function of these proteins must now be determined Many proteins are components of homomeric or heteromeric protein complexes and their activity depends on the presence of the other polypeptides in the complex (Alberts, 1998). Protein complexes are further organized in to pathways and interact with other macro molecular complexes. In addition, the composition, stoichiometries, and structures of complexes can be influenced by environmental change. Thus, to correctly determine the functions of all gene products and how they are regulated, it is essential to identify the interactions between individual proteins and thoroughly characterize complexes.

Two principle methods have been used to identify the physical interaction between proteins on a genome wide basis: two hybrid screens and TAP followed by mass spectrometry. Each method has its strengths and weaknesses.

Two hybrid screens are a genetic assay that measures the interaction of two proteins expressed as heterologous fusions in yeast cells. These screens have a higher throughput than TAP and can detect transient interactions with disassociation constants in the μM range. But they cannot detect interactions that require more than two proteins; they have a false positive rate between 50%-90%, even when testing yeast proteins in yeast cells (van Merring et al, 2002; Edwards et al, 2002); and they do not give rise to a pure sample of protein complex.

TAP is a biochemical method in which a protein subunit is tagged with two separate affinity tags separated by a protease cleavage site (Puig et al, 2001). The tagged protein is expressed in vivo at natural or close to natural levels and—after cell lysis—complexes with the double tagged protein are purified over two affinity columns, resulting in very pure complex preparations. The identity of the co-purifying polypeptides is then determined by mass spectrometry. While this method cannot detect protein/protein interactions in the μM range, it has been used to detect hundreds of stable protein complexes in yeast and E. coli (Butland et al, Nature 2005, 433, 531-537). Compared to a curated set of known protein complexes, only 15% of expected interactions were not found in an analysis of about ¼ of the yeast proteome (Edwards et al, 2002), and the false positive rate of this method is regarded as being lower than that for two hybrid screens. To date this method has been limited to the analysis of heteromeric complexes, but with the addition of a simple characterization step, it could be used to detect higher multimeric homomeric complexes as well.

Despite the strength of TAP, it suffers from three deficiencies that are especially problematic given the needs of the DOE's Genomics: GTL project.

The DOE has identified a range of bacteria whose molecular pathways and regulatory networks it wishes to enumerate and model, which will in turn open the way for the use of these organisms for bioremediation and energy production. Many of these organisms, however, cannot yet be modified by genetic or recombinant techniques, and thus a method that does not require genetic/molecular manipulation of the organism would be a great advantage.

The TAP strategy requires that for each protein tagged, a separate strain of bacteria must be cultured, extract prepared, and protein purified, which is intrinsically labor some.

A major objective of the Genomics: GTL program is to characterize the changing interactions between proteins as the local conditions experienced by bacteria alter, and a key part of this projects goal is to analyze stress induced changes in complexes. To compare changes between environmental conditions for many complexes would require enormous precision and reproducibility between growth conditions used for each strain that would be difficult to achieve.

Thus, there is a need for a different approach that does not require the use of affinity tags, has the potential of much higher throughputs, and purifies complexes from a single large culture of cells.

Historically, stable protein complexes were identified one at a time, often as the result of purifying an enzyme activity of interest. In this traditional approach, complexes were inferred when multiple polypeptides co-migrated together with an associated enzyme activity through multiple chromatographic separation steps,²⁻⁴ demonstrated the same sedimentation velocities^{5,6} or electrophoretic mobilities.⁷ More recently, stable protein complexes have been identified using high throughput mass spectrometric detection of collections of polypeptides that are stably associated with heterologous affinity-tagged polypeptides.⁸ In particular, tandem affinity purification⁹⁻¹² has proven to be highly effective in mapping the soluble portion of the yeast^{13, 14} and E. coli¹⁵ interactomes. Despite its undoubted utility, however, TAP suffers from several limitations. For example, this method is restricted to biological systems that are amenable to the genetic manipulations required to introduce the affinity tagged polypeptides into cells. Furthermore, the addition of an affinity tag may destabilize some protein-protein interactions or alter other relevant protein activities. Finally, TAP requires a distinct genetic strain to be constructed for each polypeptide and then each strain must be separately cultured and analyzed. These and other limitations suggest that it may prove difficult to automate this strategy to achieve higher throughput than has been already attained

### BRIEF SUMMARY OF THE INVENTION

The present invention provides a method of protein separation and analysis and a multi-channel gel electrophoresis instrument, capable of high resolution separation, and fast and continuous fraction collection over broad mass or size ranges. The invention employs a strategy of using multiple, short linear gels to achieve separation power similar to a long gradient gel. The fraction is then eluted in continuous and parallel fashion. The method works particularly well on SDS-gels.

Thus, in one aspect, a multi-channel gel electrophoresis apparatus for efficiently collecting molecules isolated by gel electrophoresis so they can be further analyzed, identified, or used as reagents or medications. The device collects biomolecules (protein, DNA, RNA, or pieces thereof) as they migrate off the bottom of gels. It uses a combination of fluid dynamics, electromotive forces, and gravity to increase the efficiency, concentration, and speed at which the bands of molecules are eluted into collecting wells. The device uses multiple parallel channels, harvests molecules at an efficiency of 50% or more, and has been scaled-up and automated for high-throughput. The eluted fractions are delivered directly into multiwell plates, where the molecules can be digested, if necessary, and directly analyzed by mass spectrometry or other techniques. The method can be used in native (or denatured) protein electrophoresis to analyze protein complexes in biological systems.

In one embodiment of the instrument, for a typical 2.5-hour electrophoresis run, each sample can be separated and eluted into 48 to 96 fractions over the mass range from ˜10 KD to 150 KD; sample recovery rate can reach 50% or higher; each channel can be loaded with up to ˜0.5 mg material in a 0.5 mL volume and a purified band typically elutes over 2-3 fractions (200 μl/fraction). For native gel electrophoresis, the sample loading capacity may be limited to ˜50 μg per channel due to protein aggregation. The system can however be used for native gels where some aggregation and dilution are tolerable.

A method for biomolecule size separation using electrophoresis comprising (a) providing a polymerized electrophoresis gel loaded with the biomolecules to be separated and purified; (b) performing electrophoresis on said gel to separate the biomolecules; (c) capturing the separated biomolecules as they migrate off the gel.

The methods provided by the present invention are high throughput methods and can be implemented in production pipelines to rapidly purify and identify the majority of stable protein complexes in a cell. Protein complexes or “molecular machines” perform a variety of discrete and highly specialized processes that modify and dictate systems molecular states, which, in turn, define cellular physiology in response to genetic and environmental stresses. To systems biology, it is essential to identify and characterize the complete inventory of protein complexes in high throughput

The invention further provides a method to rapidly purify and identify the majority of stable protein complexes in a cell without the use of affinity tags or affinity purifications. The “tagless” approach includes taking a crude protein extract prepared from a single large culture of cells, sequentially fractionating the extract by a number of orthogonal chromatographic separation steps (using ion exchange column, hydrophobic interaction column (HIC), sizing-column or native gel electrophoresis, for example). Selected fractions from each column are used as the input of the next step. At the last step, there are several hundreds of parallel sizing or native gel electrophoresis runs, generating 10,000-20,000 fractions that are proteolyzed prior to labeling the peptide products. Identifications of protein contents and their relative abundances in these fractions are obtained by tandem mass spectrometry (MS/MS). Protein complexes are inferred by analyzing elution profiles of all proteins detected and discovering proteins that co-migrate in the multi-fractionation space. In principle, this approach is suitable to detect endogenous protein complexes from wild type cells based on the shared elution profiles of polypeptides that, as components of a protein complex entity, co-migrate through multiple chromatographic steps.

The invention further provides a high throughput native gel system. Such as system shows promise that it can be incorporated into a “tagless” approach to protein purification and isolation to replace the need for a sizing-column, which is inherently low throughput and low resolution.

Tandem affinity purification is the principle method for purifying and identifying stable protein complexes system wide in whole cells. Although highly effective, this approach is laborious, prone to artifacts, and impractical in organisms where genetic manipulation is not possible. Herein is described a novel “tagless” method that combines multi-dimensional separation of endogenous complexes with mass spectrometric monitoring of their composition. In this method, putative protein complexes are identified based on the co-migration of collections of polypeptides through multiple orthogonal separation steps.

A majority of E. coli proteins are shown to remain in stable complexes during fractionation of a crude extract through three chromatographic steps. The inventors also demonstrate that iTRAQ™ reagent-based tracking can quantify relative migration of polypeptides through chromatographic separation media. LC MALDI MS and MS/MS analysis of the iTRAQ-labeled peptides gave reliable relative quantification of 37 components of 13 known E. coli complexes: 95% of known complex components closely co-eluted and 57% were automatically grouped by a prototype computational clustering method.

The assay and method of the invention dramatically improves the efficiency of the purification and identification of protein complexes in cells. In fact, the assay itself, being essentially a single experiment with two sequential procedures (separating co-migrating complexes and analyzing each complex for protein content) allows the capture of information about whole cells that was previously unattainable without months of laborious experiments.

Accordingly, the invention provides an opportunity to generate a database of the protein complexes present (as identified by this method and this assay) in any number of whole cells from any source (e.g. cell lines, tissue, plants and all other sources of whole cells). Presently, the only cell type for which this information is available is a yeast cell, and the information to map the entire protein complex profile of the yeast cell took many many months of experiments and data collection.

A high throughput method of identifying protein complexes in whole cells from any organism comprising: (a) passing cell lysate from whole cells through at least two orthogonal separations under conditions that preserve interactions among polypeptide components of protein complexes in the lysate, (b) collecting polypeptide components in separate elution fractions, (c) proteolytically digesting each fraction separately to produce a plurality of peptides; (d) analyzing the peptides in each fraction for peptide identity and abundance of the peptide in the fraction relative to the other fractions, and (e) identifying co-migrating polypeptides using mathematical analysis based on peptide distribution in the fractions, wherein co-migrating polypeptides identify protein complexes in the cell.

The peptides in each fraction for protein identity and abundance relative to the other fractions may be analyzed by mass spectrometry. In another embodiment, identifying co-migrating polypeptides comprises clustering. In another embodiment, passing cell lysate from whole cells through at least two orthogonal separations comprises passing the cell lysate through a chromatographic separation. In another embodiment, the determining of a structure of at least one protein complex may be accomplished by electron microscopy.

The high throughput method further comprising storing protein complex information from a plurality of whole cells in an interactive database accessible to a plurality of users, wherein the protein complex information comprises substantially all the protein complexes in the cell. In another embodiment, the method further comprising storing monomer and other single protein information.

A high throughput method of identifying protein complexes in whole cells from any organism comprising: (a) providing whole cells, (b) separating cell lysate from said whole cells under conditions that preserve interactions among polypeptide components of protein complexes in the lysate, (c) collecting said polypeptide components in separate elution fractions, (e) analyzing the peptides in each fraction for peptide identity and abundance of the peptide in the fraction relative to the other fractions, and (f) identifying co-migrating polypeptides using mathematical analysis based on peptide distribution in the fractions, wherein co-migrating polypeptides are protein complexes in the cell

From the method described in this invention, any number of researchers could develop a database that stores the information learned by practicing the tagless method of identifying protein complexes in a cell. For example, the database could classify cells by listing the protein complexes found by the method, and reference details of the isolation procedure (e.g. how many and which orthogonal separations were conducted). The database could, in addition, provide a platform for comparing cells, e.g. such as diseased and healthy cells, cells from different tissues, and search on given protein complexes to learn in which cells they are found. Clearly such information is vastly useful in planning any research using a particular cell, and for many uses relating to use of these cells, such as for diagnostic purposes and in development of therapies for treating patients with a condition that can be defined at a cellular level.

### BRIEF DESCRIPTION OF THE DRAWINGS

FIG. 1. System design and operation scheme for Counter Free-Flow electrophoresis. (a) Shows an elution unit and two separate linear gel columns (Gel segments 1 and 2). Each gel column is attached to a running buffer container that contains a Platinum electrode. The upper column (Gel segment 2) has the lowest polyacrylamide concentration gel and space above the gel for sample loading. The lower column contains a higher polyacrylamide concentration gel. (b) Stacking Mode. Shows these three pieces stacked to form an approximation of a gradient gel. Gel segment 1 is inserted into a conduit in elution unit 1, and Gel segment 2 is stacked on top of this. Then, an initial phase of electrophoresis is used to separate proteins from a complex mixture loaded at the top of Gel segment 2. High mobility protein bands migrate into the Gel segment 1 while slow ones remain in Gel segment 2. (c) Single Mode. Illustrates that, after separating (un-stacking) the two gel columns, the buffer container associated with Gel segment 1 is filled, while Gel segment 2 is inserted into a conduit in elution unit 2 (see text and FIG. 1d). Finally, electrophoresis is resumed on both gel columns to further separate and elute the protein bands captured in each gel segment. To stack more than two gel segments in a separation, additional gel boxes designed like Gel segment 1 can be included because they are constructed to allow “self-stacking”. (d) Alternative Single mode. As illustrated, three pieces of linear gel columns of different concentration can be inserted into three gel segment units and stacked to form a single gradient gel. After an initial phase of electrophoresis, protein bands of complex mixture migrate into different segments of the gel column and top two pieces of gel columns can be moved to two different elution units. Continuing electrophoresis, protein bands captured in each segment of gel can be further separated and eluted simultaneously.

FIG. 2A shows a single elution unit. with a single gel column inserted (for clarity the associated upper buffer reservoir is not shown). A capillary tube with a sleeve of PEEK tube is attached to the base plate of the lower (anode) buffer reservoir. Buffer is added to completely fill the reservoir. A conduit of acrylic glass is then inserted from above, fitting over the capillary and PEEK tubes. The tight fit between the PEEK tube and the lower portion of the conduit ensures that the capillary is centered. A gel column consisting of a vertical glass column containing polyacrylamide gel is then inserted down into the conduit as far the taper. Care is taken to avoid trapping air bubbles between the gel column and the taper. After insertion, the buffer level is lowered and maintained at the level indicated by a slow gravity fed inlet and outlet flow so that the electrical contact to the gel is established only through the conducting holes. The elution unit is shown in relation to the fraction collection plate and controlled stage. FIG. 2B shows a multi-channel elution unit with the fraction collection unit. FIG. 2C shows a multi-channel elution unit with a fraction collection unit enclosed in a back pressured container.

FIG. 3. A photograph of the prototype 16-channel Counter Free-Flow electrophoresis and elution apparatus. It includes gel boxes that each house four gel columns of either 3- or 5-cm length (top), four elution units (middle) and two motorized fraction collectors (bottom)

FIG. 4A is a time series of photographs showing the channels during elution and capture of bio-molecules migrating off the bottom of gel column. As shown, blue bands of dye molecules are about to emerge from each lane. When the parameters are properly set, the efficiency capturing bio-molecules off the gel column could be high. In the insert at the lower part, for example, sequential five snap-shots of lane #2 (from left), taken during 3 minutes span, illustrate that most dye molecules were captured by capillary tube. Notice that the dye molecules were sharply focused around the tip means they were drained; and the blue “cloud” was restricted above the tip indicates there was no significant “leakage.

FIG. 4B shows images of gels showing separating and eluting pre-stained protein markers from a two stage multi mode SDS Counter Free-Flow gel. All 10 pre-stained marker protein bands were clearly separated by two short SDS gels and eluted into 2×24 fractions. The fact that the four smaller molecular weight polypeptides (10-25 kD) eluted over the first 7 fractions and that the 37 kD band eluted over only two demonstrate the effectiveness of the second stage 6.5% gel over this size range. The first stage 4.5% gel was similarly effective for the 75 and 100 kD bands. However, the spread of the 50 kD and 150 kD bands over 5 fractions each also illustrates that sample dilution occurs as protein mobility moves out of the gel's optimum fractionation range. The fractions for each column were collected sequentially from top (left), down, then back up (right) at 2 minutes per fraction.

FIG. 4C the order of fractions collected by the actuation-controlled fraction collector stage and controls.

FIG. 5 shows elution of SDS Counter Free-Flow PAGE gels. Left panels (a, b, c): protein contents of collected fractions of a crude extract of D. vulgaris separated using the single mode and different gel concentrations. Right panels (d, e, f): results obtained using two-staged “multi” mode and different gel combinations. The concentrations in the gel columns used are given in each panel. The contents of each eluted fraction were visualized via slab SDS-PAGE and silver staining. The sample input lane is marked as “I,” protein markers as “M” and other lanes according to the eluted fraction number.

FIG. 6A-C are images of gels showing separation characteristics of short linear native gels of 4, 6 and 8%. Separation characteristics of linear native gels of 4, 6 and 8% run in single mode Counter Free-Flow PAGE. The contents of each eluted fraction are visualized via slab native PAGE and silver staining. The lanes are numbered as in FIG. 5.

FIG. 7. Native Counter Free-Flow PAGE induces protein aggregation. A 5% gel, single mode Counter Free-Flow electrophoresis of a desalted and concentrated HIC fraction with lower complexity of proteins than a crude extract. Eluted samples were analyzed by (a) native slab gel and (b) SDS slab gel, showing the major protein contents in each fraction. The input samples labeled I and I′ are HIC fractions desalted but before and after concentration by a 3-kD column, respectively.

FIG. 8. Multi mode run of Native Counter Free-Flow PAGE. Gels of 5% and 8% were used to separate a crude extract of D. vulgaris in the multi mode. The contents of each eluted fraction are visualized via slab native PAGE and silver staining.

FIG. 9. Elution of native PAGE gels. (a): a 5% and 8% “stacked” run of crude extract of D. vulgaris; (b): a 5% gel, “direct” run of desalted and concentrated HIC fraction; (c): corresponding SDS gel of (b) shows major protein contents in each fraction collected; (d): a 8% “direct” run of HIC sample; and (f): corresponding SDS gel of (d). The input samples labeled “I” and “I′” are HIC fractions desalted but before concentration and after concentration by 3 Kd column, respectively.

FIG. 10 shows capturing bio-molecules migrating off the bottom of gel column. As shown, blue bands of dye molecules are about to emerge from each lane. When the parameters are properly set, the efficiency capturing bio-molecules off the gel column could be high. In the insert at the lower part, for example, sequential five snap-shots of lane #2 (from left), taken during 3 minutes span, illustrate that most dye molecules were captured by capillary tube. Notice that the dye molecules were sharply focused around the tip means they were drained; and the blue “cloud” was restricted above the tip indicates there was no significant “leakage.

FIG. 11 is a schematic showing the concept of the “tagless” protein complex identification strategy. A crude cell lysate is fractionated successively by highly parallel, orthogonal purification steps: in the example given ion exchange (IEX), hydrophobic interaction (HIC) and gel filtration chromatography. A rational sampling of fractions from the preceding separation step is submitted to the following separation step, generating thousands of fractions at the last purification step. Selected fractions from the last step are then subjected to proteolytic digestion and iTRAQ reagent labeling, and the products are then analyzed by mass spectrometry to identify polypeptides and measure their relative abundances as they migrate through the separation media; the iTRAQ reagent serves as a quantitative beacon of protein presence. Similarities among polypeptide elution profiles are evaluated using clustering analysis. Putative complexes are defined as sets of polypeptides that cluster at an experimentally established confidence level. In the example shown, two putative heteromeric complexes A and B, composed of three and two co-eluting components, respectively, are shown. Two proteins with no co-eluting partners are also detected. Since the last step separates based on molecular weight, it can be determined if these non co-eluting polypeptides are either monomers or homomeric complexes

FIG. 12. Typical protein size distribution in partially fractionated E. coli lysates. The results of the SDS PAGE (5-20%) and native PAGE (5-15%) analyses of selected fractions of a crude lysate after separation by Mono Q chromatography are shown in panels A and B, respectively. Panel C shows the results of the Native PAGE (5-15%) analysis of fractions collected following gel filtration chromatography (Superdex 200 column) of one the Mono Q column fractions, annotated with an asterisk in Panels A and B. Panel D shows the distribution of total protein concentration across the Superdex200 column, the red arrow marks the estimated position of elution of a 100 kDa species. Both Native PAGE and gel filtration data suggest that the majority of protein, by mass, participates in complexes. A comparison of Native PAGE of the protein fraction annotated with an asterisk in panel B and products of its further separation (panel C) shows that the proportion of high versus low molecular weight species changes little during size exclusion chromatography, indicating that there is only slight dissociation of complexes during this step.

FIG. 13. The experimental workflow of protein identification and quantification. Eluted proteins are sampled at a frequency dependent upon the resolution of the separation step. In the example shown, every other fraction is sampled. The appropriate volume of each fraction is withdrawn so that each fraction is represented in the final four-plex set by the same amount of total protein (˜20 μg). Proteins from each fraction are independently digested with trypsin and labeled with iTRAQ reagent. Four successive fractions, each labeled with a different iTRAQ reagent are combined to form multiplexes, annotated as A, B, C and D at the bottom of the Figure. Each pair of adjacent multiplexes shares one bordering fraction. Each multiplex is analyzed by a reversed phase nanoLC MALDI MS and MS/MS. Elution profiles are generated for each detected protein on the basis of iTRAQ-derived relative abundances within all multiplexes, as described in the Methods

FIG. 14. iTRAQ analysis of co-migrating RNA polymerase subunits. Panel A shows the result of SDS PAGE of a subset of fractions from a Mono Q column elute. The positions of components of RNA polymerase complex are annotated with arrows, of which RpoB, C and A are visible within the mixture of other proteins. Two overlapping four-plexes (Set A and Set B) that shared fraction E5 (annotated with an asterisk) were generated by combining every other fraction labeled with an iTRAQ reagent. Panel B shows the relative abundance of each of the different tryptic peptides from RpoA derived after iTRAQ analysis (thin grey lines). The thick red line represents the mean elution profile for RpoA based upon the average for all peptides. Although there is some peptide to peptide variation, they all closely approximate the mean. In panel C, the mean elution patterns for the five major polypeptide components of RNAP complex are shown. All closely co-migrate under the conditions of this experiment, suggesting that iTRAQ-derived mean relative abundances confidently represent protein elution profiles. For all profiles in panels B and C, the fractions with the maximum levels of peptides are set to a nominal relative abundance of 1.0.

FIG. 15. Reproducibility of tryptic digestion, iTRAQ labeling, and LC MALDI MS/MS analysis. A subset of Mono Q column fractions (see Panel A in FIG. 4) was analyzed in duplicate by independently performing tryptic digestion, iTRAQ labeling, and LC MALDI MS/MS analysis. The mean iTRAQ elution profiles of the three components of pyruvate dehydrogenase complex (LpdA, AceE, AceF) derived from the two experiments are very similar. Panels A and B demonstrate the reproducibility of the methods.

FIG. 16. Reproducibility of chromatographic separation. Mean iTRAQ elution profiles of the polypeptide components of RNA polymerase (RpoA, RpoB, RpoC, RpoD), pyruvate dehydrogenase (LpdA, AceE, AceF) and 2-oxoglutarate (SucA, SucB, LpdA) during anion exchange chromatography in two independent protein separation experiments. Panel A shows the results from the same Mono Q fractionation shown in FIGS. 4 and 5. Panel B show the results from a larger scale Mono Q separation of a different crude extract preparation. The order of elution of complexes in the two different Mono Q experiments is the same, testifying to the feasibility of comparing results between parallel columns at the same step of a tagless fractionation of a single extract or between equivalent column fractionation of two closely related protein extracts.

FIG. 17. The effectiveness of clustering in grouping complex components. Panel A shows the mean iTRAQ profiles of the five main components of RNA polymerase (RpoA, RpoB, RpoC, RpoD, RpoZ) and also the transcription termination/antitermination factor NusA and transcription termination factor Rho. Panel B shows the constituents of a cluster defined by our algorithm that includes four of the five main RNA polymerase subunits and Nus A, but not RpoZ and Rho. The cluster also contained proteins that had likely fortuitously co-eluted with RNA polymerase: YbbN, MetK, GroL and AldA.

FIG. 18. A process of generation of a polypeptide elution profile using LpdA as an example. The ordinate values correspond to the relative abundances of the component polypeptides in each fraction and the abscissa values represent the order in which the fractions eluted. Polypeptide elution profiles are derived from the iTRAQ-based average relative abundance of each polypeptide within each of the separately analyzed four-plexes. Panel A. Relative ratios for the polypeptide abundance in each fraction of each four plex is shown in a different color. Panel B Starting from the beginning of the chromatogram, pairs of adjacent mutliplexes are aligned using the fractions that are shared between them (annotated by an asterisk on the abscissa) as common join equilization points. As a result, five independent partial elution profiles as seen in Panel A are collapsed into a single elution profile with a relative abundance ratio for each data point (fraction) referring to the same reference fraction. In this case, F10 (marked by a red arrow) was arbitrarily used as the reference point. Panel C. The data are normalized by arbitrarily assigning a value of 1.0 to the fraction that contains the highest amount of the polypeptide (the highest relative abundance value) within its contiguous elution chromatogram, in this case fraction I13 (marked by a red arrow).

FIG. 19 Elution profiles of the detected polypeptide components of thirteen known protein complexes defined in the EcoCyc database. The elution profile of each polypeptide component of a protein complex is shown in different color. The number of peptides detected in the apex fraction for each polypeptide is shown in brackets after the polypeptide code name. For the sake of clarity of presentation, apex values for some of the closely co-eluting polypeptides were slightly altered from their actual value of 1.0. The information on TAP-derived interactions of polypeptides detected in this study refers to the work of Butland et al.¹⁵

FIG. 19A. The two DNA gyrase subunits GyrA and GyrB are shown. The DNA gyrase complex is known to be unstable³³ and, in our hands, GyrA and GyrB did not migrate together through the Mono Q column chromatogram. Reciprocal TAP analysis, however, did detect an interaction between GyrA and GyrB. DNA gyrase is one of the three cases in our current analysis where TAP identified an interaction that the tagless pilot experiment did not.

FIG. 19B. The two components of the Tol-Pal Cell Envelope Complex, TolB and PaL, are shown as well as the very abundant outer membrane lipoprotein (Lpp) that is known to interact in vivo with TolB (http://biocyc.org/ECOLI/NEW-IMAGE?type=ENZYME&object=EG11008-MONOMER). TolB, PaL and Lpp proteins clustered using algorithm employed in this study. Pal protein was also detected in two other stretches of the Mono Q column fractions, possibly indicative of its independent interaction(s) with other partners that were not detected. As described in the body of the paper, a quantitative comparison among relative abundances of a polypeptide within disparate segments of a non-contiguous elution profile cannot be made; hence, each local apex was arbitrarily assigned a value of 1.0.

FIG. 19C. All three known components of the DNA K system complex, DnaJ, DnaK, and GrpE were detected. These polypeptides displayed complex but highly overlapping elution profiles, possibly indicating the presence of multiple sub complex forms. TAP analysis detected reciprocal interactions between DnaK and GrpE and one way interaction between DnaJ->DnaK but no DnaJ-GrpE interactions.

FIG. 19D. Two of the ATP synthase F1 complex subunits, AtpA and AtpD, were detected. They shared an apex of elution and belonged to the same cluster. In TAP analysis AtpA, but not AtpD, was utilized as a bait and its interaction with AtpD was detected.

FIG. 19E. The DNA polymerase III holoenzyme subunits DnaE, HolE and DnaX were detected and belonged to the same cluster. In the TAP analysis, DnaE<->HolE and DnaX<->HolE were detected as reciprocal interactions while DnaE->DnaX was detected as a one-way interaction where DnaE served as a bait but not with DnaX as a bait.

FIG. 19F. Both of the known components of the flavin reductase/sulfite reductase-(NADPH) complex, CysJ and CysI, were detected and showed very similar elution profiles. Neither polypeptide was used as a bait in the TAP study.

FIG. 19G. The five main components of RNA polymerase were detected, RpoA, RpoB, and RpoC RpoD and RpoZ, together with the transcription termination/anti termination factor NusA and transcription termination factor Rho. As discussed in the main text, these components had similar profiles, but NusA and Rho appear to migrate more sharply, perhaps reflecting the chromatographic properties of a sub form of the RNA polymerase complex. Interestingly, orthogonal TAP analysis detected all these components interacting with each other in a reciprocal manner except for Rho, which was only seen as a prey interacting with RpoA, RpoB and RpoD but not with RpoC.

FIG. 19H. All three components of the chromosome partitioning complex, MukB, MukE and MukF, were observed. MukE and MukF co-elute in broad peaks, whereas MukB forms a somewhat narrower chromatographic peak. Reciprocal interactions between MukE and MukB were detected by TAP, as was a one way interaction between MukB->MukF; MukF was not used as a bait in the TAP study.

FIG. 19I. All three components of pyruvate dehydrogenase, LpdA, AceF, and AceE, were detected. Their elution profiles were highly similar and shared the same elution maxima. LpdA and AceE, however, also showed smaller secondary elution peaks in other parts of the MonoQ chromatogram. Neither of these proteins was examined as a bait in the TAP study.

FIG. 19J. All three components of 2-oxoglutarate dehydrogenase, LpdA, SucB, and SucA were detected. All three polypeptides displayed two elution maxima, one of them overalapping with that of pyruvate dehydrogenase complex (Panel I). TAP analysis detected reciprocal interactions between SucA and SucB while LpdA (not utilized as a bait) was observed as a prey with both SucA and SucB serving as baits.

FIG. 19K. Two components of the Glycine cleavage complex, LpdA and GcvP, were identified and albeit they were detected in the same fractions, they did not share the apices of elution. The elution maxima of LpdA in fractions H10 and I13 is explained by its participation in two additional complexes: pyruvate dehydrogenase (panel I) and 2-oxoglutarate dehydrogenase (panel J). Further study is required to verify whether the presence of LpdA and GcvP in the same fractions is caused by their interactions. Neither GcvP nor LpdA were studied as baits by TAP.

FIG. 19L. The two components of Aspartate carbamoyltransferase, PyrB and PyrI, were both detected and their elution profiles clustered. The shape of the elution peak suggests that the complex elutes with at least two maxima, with the major one outside the examined fraction set of Mono Q column eluate. PyrI and PyrB were not used as baits in the TAP study.

FIG. 19M. Both subunits of glycyl-tRNA synthetase, GlyS and GlyQ, were detected and eluted within the same fractions but their elution profiles did not overlap. Interaction between GlyS and GlyQ was not detected in the TAP study.

FIG. 20 SDS PAGE analysis of E. coli protein complexes purified using the tagless strategy. The purification scheme consisted of two common steps: gel filtration (Sephacryl S400 320 mL column) and anion exchange chromatography (Mono Q, 20 mL column) that were followed either by another gel filtration step (Superose 6, 24 mL column) for pyruvate dehydrogenase (Panel A) or hydrophobic interaction chromatography step (Source 15HPC, 1.7 mL column) for RNA polymerase (Panel B) and 60 kDa chaperonin (Panel C).

FIG. 21. Tagless survey of large D. vulgaris protein complexes that bind to Q-Sepharose resin from a 400 L culture preparation. MonoQ column fractions from 6 ammonium sulfate precipitation cuts were each analyzed by native PAGE (4-15% acrylamide): ammonium sulfate saturations of A. 0-38%; B. 38-48%; C. 48-53%; D. 53-57%; E. 57-63%; F. greater than 63%. Arrows show the 14 protein complexes that were sufficiently purified for EM analysis after further fractionation: 1. Putative protein (DVU0631); 2. Phosphorylase (DVU2349); 3. Hemolysin-type calcium-binding repeat protein (DVU1012); 4. Phosphoenolpyruvate synthase (DVU1833); 5. Proline dehydrogenase/delta-1-pyrroline-5-carboxylate dehydrogenase (DVU3319); 6. Pyruvate carboxylase (DVU1834); 7. Inosine-5′-monophosphate dehydrogenase (DVU1044); 8. RNA polymerase (DVU1329, DVU2928, DVU2929, DVU3242); 9. Predicted phospho-2-dehydro-3-deoxyheptonate aldolase (DVU0460); 10. Putative protein (DVU0671); 11. Ketol-acid reductoisomerase (DVU1378); 12. Pyruvate-ferredoxin oxidoreductase (DVU3025); 13. 60 kDa chaperonin (GroEL, DVU1976); 14. Riboflavin synthase (DVU1198, DVU1200).

FIG. 22. Tagless survey of large D. vulgaris protein from a 400 L culture preparation that did not bind to Q-Sepharose resin. These proteins were analyzed by SEC and the fractions then separated by SDS PAGE. The arrow shows alcohol dehydrogenase, which was sufficiently purified for EM analysis by further fractionation. Size markers for the SDS PAGE are shown at the left of the gel and the positions of size makers on the SEC column are shown at the top of the gel.

Table 3: Non-Ribosomal Proteins Identified by in the Course of Tagless Protein.

Table 4: Tagless Strategy-Detection of Reciprocal Protein-Protein Interactions Previously Identified by TAP.

Table 5: Results of Clustering Analysis of Elution Profiles of Non-Ribosomal Proteins

Table 6. Biochemical identity and composition of large macromolecular complexes purified from Desulfovibrio vulgaris Hildenborough by the tagless strategy. Homologs from other bacteria listed in the rightmost column are members of the same Pfam families as the D. vulgaris protein.

### DETAILED DESCRIPTION OF THE PREFERRED EMBODIMENT

### Introduction

Important issues that needed to be resolved in fraction collection included preventing interruption of electrophoresis, maintaining the continuity of collection, and reducing sample loss, resolution loss, and dilution of protein bands.

To facilitate a direct interface between protein separation and isolation by polyacrylamide gel electrophoresis and protein identification by mass spectrometry, a multi-channel system was developed for continuous fraction collection as protein bands migrate off the bottom of gel columns. It was constructed based on a scheme that uses multiple short linear gel columns to achieve separation power similar to a long gradient gel, and an elution technique that allows continuous and simultaneous fraction collections of multi-channels at low costs. Fast and high resolution separation and fractionation of complex protein mixture can be achieved on this system while running SDS-PAGE gels.

In a 2.5-hour electrophoresis run, for example, each sample can be separated and eluted into multiple (e.g., 48 to 96) fractions over the mass range of ˜10 KD to 150 KD; sample recovery rate can reach 50% or higher; each channel can be loaded with up to 0.5 mg material in 0.5 mL volume and a purified band typically elutes over 2-3 fractions (200 μl/fraction). Similar results could be obtained when running native gel electrophoresis on this instrument, but protein aggregation, mainly caused by sample over-loading and stacking, may limit the loading capacity to about 50 μg per channel.

### DESCRIPTIONS OF THE EMBODIMENTS

The goal of this work was to develop an easy to use multi-channel system that is effective in separating a complex mixture of proteins while automatically capturing separated bands into a liquid fraction collector, over a broad mass/size range. Such a system can be built based on a simple scheme.

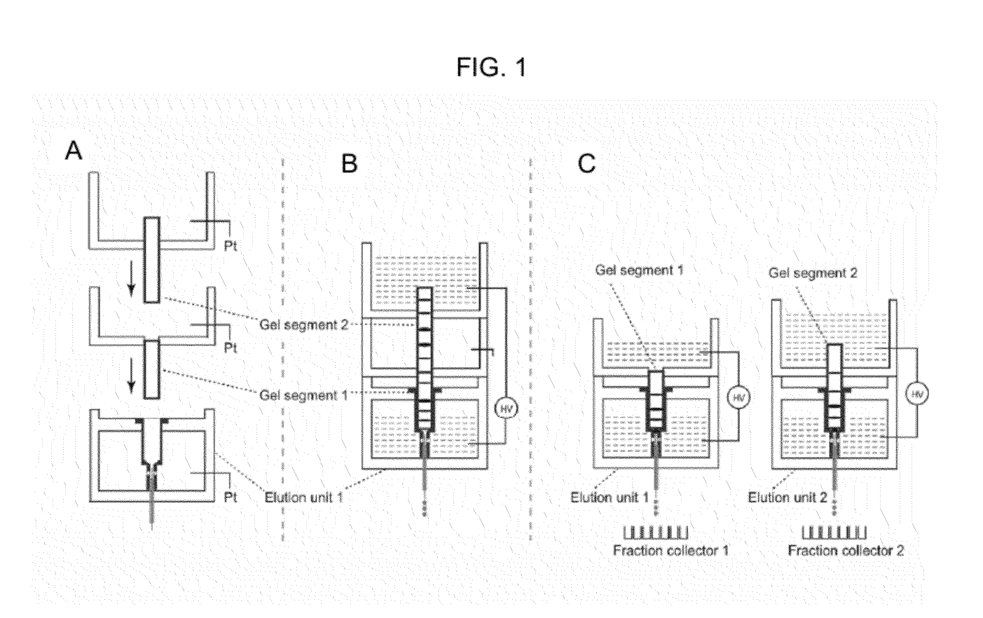

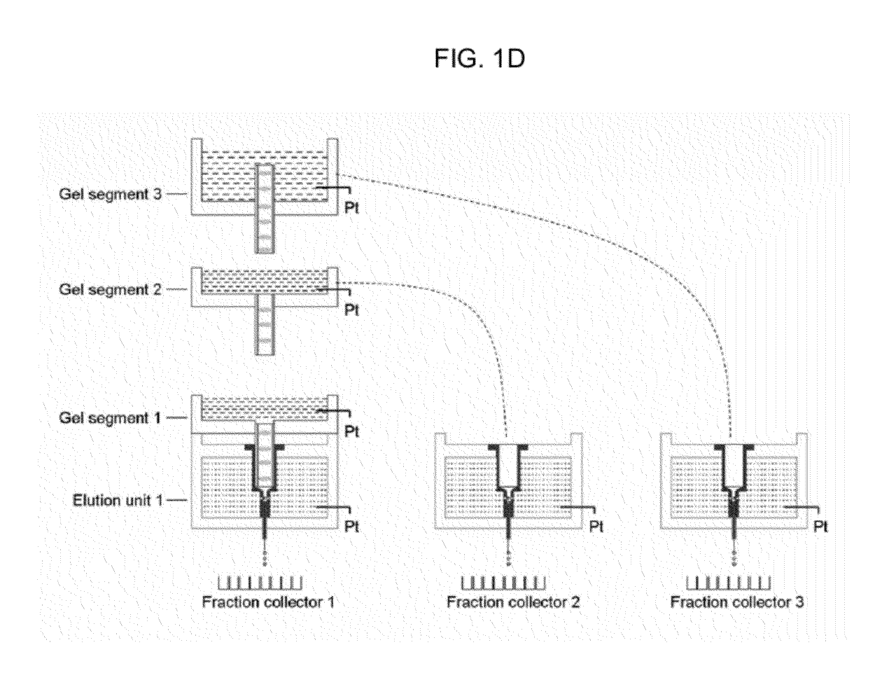

FIG. 1 illustrates an example of such a scheme. As illustrated, three pieces of linear gel columns of different concentration can be stacked to form a single virtual gradient gel column. After an initial phase of electrophoresis, protein bands of complex mixture migrate into different segments of the gel column and top two pieces of gel columns can be moved to two different elution units. Continuing electrophoresis, protein bands captured in each segment of gel can be further separated and eluted simultaneously.

In one embodiment, as shown in FIG. 1, two or three short (2-3 cm long) linear native 4, 6 and 8% cross-linked polyacrylamide gels are stacked in gel columns for an initial run of electrophoresis, giving rise to a separation comparable to a 4-12% gradient gel. Bands of separated bio-molecules can be subsequently eluted and collected on separate devices. This modular approach also allows a specific band of interest from the entire electropherogram to be separated and eluted by a single short gel.

As used herein, by the term, “gel column,” it is meant, the gel itself or the gel and the gel column tube or channel containing the gel together. For example, a gel column is prepared in a glass tube using a rubber stopper and a piece of thin plastic film to seal the bottom of the tube, and is referred to as a “gel column.” Thus, “gel column” can mean both the column gel itself in the gel column tube or the gel and gel column tube containing the gel.

Thus, in one embodiment, the multi-channel system as in FIG. 1A, the multi-channel system comprising (1) an elution unit having conduit or a plurality of conduits, each conduit having an upper and a lower tapered region, and further having a polymerized linear gel column in the upper region of said conduit, and at the lower region featuring conducting holes and a capillary tube inserted at the bottom of the tapered region; (2) a gel segment container having a tube length in which a gel column can be formed and polymerized, or a plurality of tube lengths, wherein the tube and the conduit fit together end to end such that the gel columns in the tube and the conduit are stacked, (3) a metal electrode connecting the elution unit and the gel segment container, and (4) running buffer to conduct electrophoresis.

The plurality of linear gel columns can each have different polyacrylamide concentrations, to achieve separation power similar to a typical gradient gel thereby enabling continuous fraction collections of multiple gel columns. In one embodiment, the lower gel column is inserted into the conduit in the elution device prior to electrophoresis, where it remains until all fractions are collected. In FIG. 1, a multi mode operation is shown. Two ˜2 cm long linear native 5 and 8% polyacrylamide gels are set in glass tubes and the resulting gel columns are stacked one on top of the other to mimic a 5-8% gradient gel. After an initial (typically, around 30 minutes) electrophoresis run, the top gel column is moved and placed on another elution unit (unit 2 in FIGS. 1C and 1D). Electrophoresis is then continued on both units to further separate protein bands. The proteins that as a result elute from the bottom of each gel column are collected in parallel on fraction collectors (FIGS. 1C and 1D).

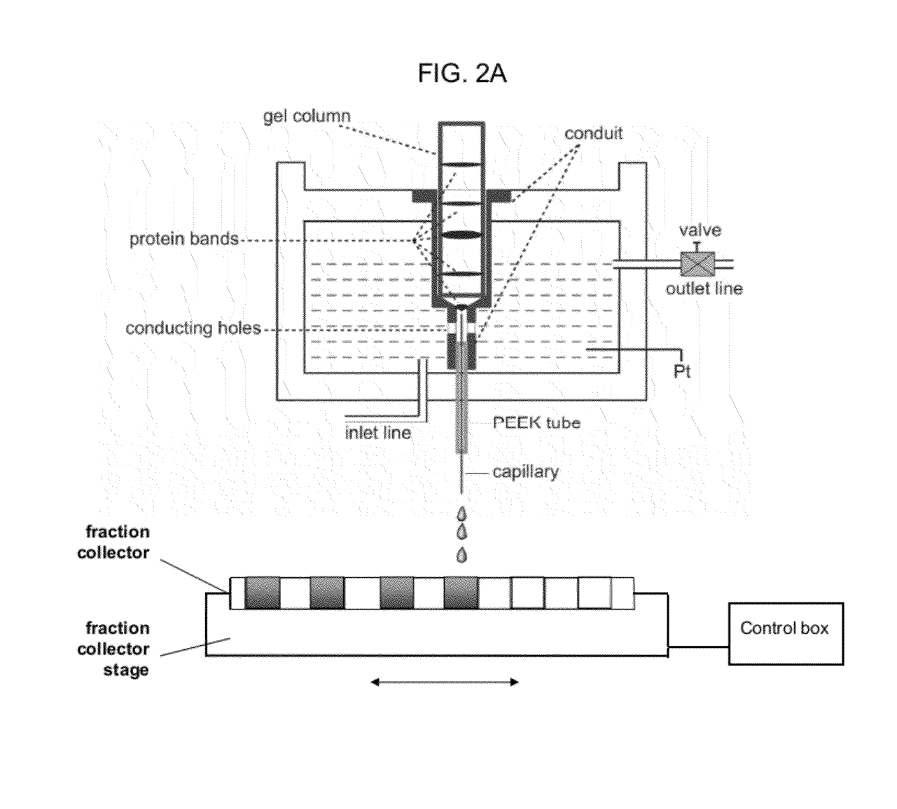

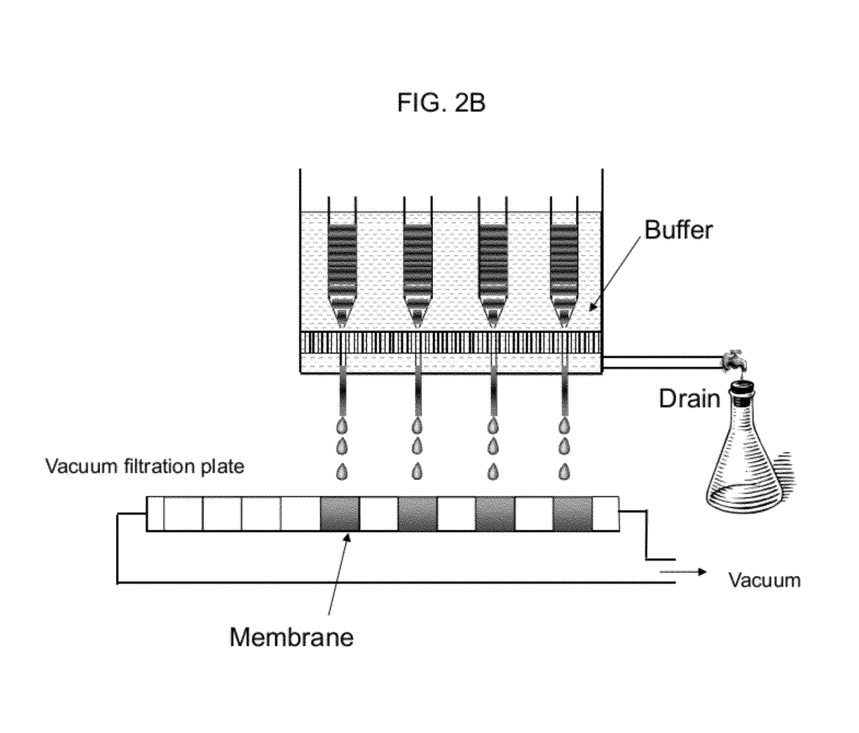

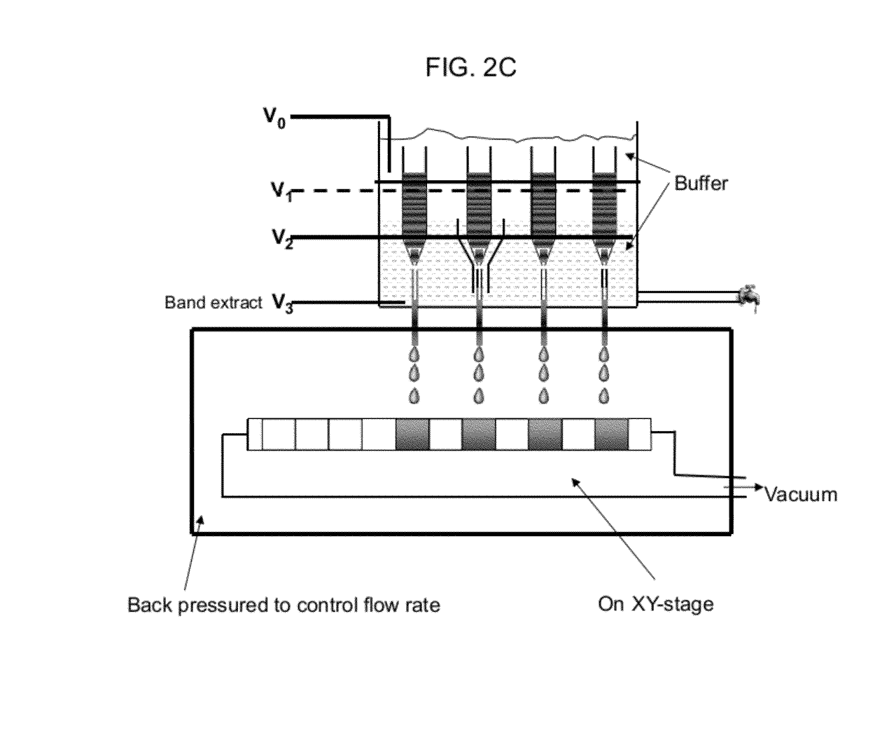

Elution Unit. Referring now to FIG. 2A, in another embodiment, the multi-channel system comprising an elution unit having a conduit with an upper region that is of sufficient width or diameter to hold a gel column which tapers to a lower region that snugly fit a capillary tube with a sleeve inserted into the conduit from the bottom end. In one embodiment, the upper region is shaped to snugly fit a gel column. The conduit is fitted onto a sidewall of a closed buffer container which has an opening on the top of the container to insert the gel column in the upper region of the conduit. The buffer container also has a small opening on the opposite sidewall to allow a capillary tube or the like to be inserted into the tapered end of the lower region of the conduit. The lower region of the conduit featuring conducting holes through which the running buffer can flow. The elution unit can be constructed with sides or a container above the conduit as a buffer reservoir.

To simultaneously capture protein bands off multiple gel columns, a “free-flow” technique was developed. In one embodiment, the elution unit comprising a machined conduit of acrylic glass and a fused-silica capillary tube. As shown in FIG. 2, the conduit provides the interface between electrophoresis and the collection of eluted biomolecules. The gel column can be easily inserted into the upper cup of the conduit and the taper (a funnel) at the bottom of the cup provides physical support to the gel. This arrangement reduces the diameter of eluting bands from 7 mm to 3 mm over a vertical distance of 2 mm as they move down the taper.

Below the taper is the final portion of the conduit, a straight tube with an outer diameter (od); and in the middle of the length of this tube, four holes are drilled perpendicular to the central axis of the tube. In one embodiment, the straight tube is 12-mm long, with a 4.5-mm outer diameter (od); in the middle of the length of this tube, four 1.0 mm diameter holes are drilled perpendicular to the central axis of the tube, and the inner diameter (id) of the straight tube is 3 mm above the holes and only 1.53 mm below the holes. These holes, termed conducting holes herein, allow electrical currents to flow between the gel column and the anode and running buffer to flow in. The increased electrical field within the straight tube provides additional acceleration to eluting biomolecules once they enter the gel-free buffer solution.

A narrow-bore glass capillary tube with a sleeve (e.g., a 50 mm long PEEK tube, 0.5 mm id and 1/16^{th} inch od) is inserted into the bottom of the conduit. The PEEK tube ends about 1 mm below the four holes and the capillary tube 2 mm below the taper. When charged molecules reach the tip of the capillary tube they are subjected to an inward drag force generated by the counter-flow of buffer solution. As the rate of the counter-flow increases, it overcomes the electrical force and sweeps the biomolecules down into the capillary tube and deposits them into a fraction collector below. The buffer flow can be gravity-driven, and thus does not require expensive pumps. The buffer flow is controlled by adjusting the length and inner diameter of the capillary tube. The relative vertical position of the capillary tube within the straight tube is an important parameter that affects the capture efficiency of eluted biomolecules (see below for further details). Using this approach, many channels can be operated simultaneously.

The present elution unit uses electrophoresis buffer solution as the media to establish electrical connection between the gel column and the ground electrode, but also the bulk flow of buffer solution to drain separated bands migrating off the gel column. The flow is gravity-driven and the rate can be controlled by adjusting the length and inner diameter of the capillary tube. The relative position of the capillary tube within the straight tube is an important parameter that affects the capturing efficiency of eluted bio-molecules (more details below).

The elution unit, which also serves as the lower buffer container, was formed by attaching a base plate holding four capillary tubes (320 μm id, 450 μm od and 15-25 cm long [part # TSP320450, Technologies, Phoenix, Ariz.]) and an O-ring gasket to a buffer container body, which includes a Pt electrode (anode) and a buffer inlet and outlet (FIG. 2 shows an example for a single gel elution unit). At the top of the elution unit, there are four precisely machined holes in which plastic conduits are inserted that receive the gel column from the segment above. A buffer inlet is attached to the bottom of the base plate so that fresh buffer is supplied to the capillaries directly. The outlet is attached to a side wall at a height that defines the level of buffer in the chamber during electrophoresis, which is about 5 mm above the taper in the conduit to ensure that the bottoms of the gel columns are submerged in buffer (FIG. 2). The buffer level is maintained either by connecting the inlet to a large (4-L) glass buffer container sitting on a lab jack that is set slightly higher than the elution unit or by using a small, inexpensive dual-channel peristaltic pump. The excess flow not consumed by the four capillaries is drained through the outlet and disposed. There is also a pinch-valve on the outlet line, which is closed to raise the buffer level to cover the tops of the conduits only when the gel columns are being inserted from above.

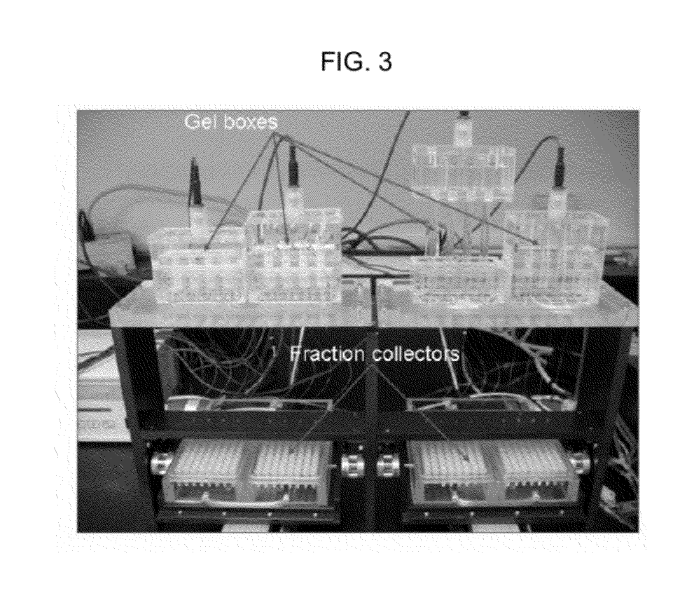

Multi-Channel Apparatus. Based on the scheme described above, a prototype, 16-channel instrument (See FIGS. 2A and 3) has been constructed and tested. We used gel segment containers that each house four short (3 cm long) glass tubes for forming the lower and middle gel segments in the “multi” mode (described below), and boxes that house four longer (5 cm or 12 cm) glass tubes for either the upper most gel segment in the multi mode or the only gel column in the “single” mode (described below) (FIGS. 1 and 3). Each box includes an upper run buffer container, which includes a Platinum electrode for use as a cathode when needed, and the four glass tubes, spaced 18 mm center-to-center (FIG. 2A shows a simpler version with just one tube per box). The bottom plate of the buffer container in each box has four precisely machined (clearance) holes. The inner diameter of the holes (at their base) tightly matches the outer diameter of the glass tubes. When the glass tubes are inserted from the bottom and glued, the tight fit ensures the tubes will be straight and evenly spaced. For the lower (short) gel boxes, the top of each tube ends about 5 mm below the top of the hole (FIG. 2A). Above this, the inner diameter of the holes is slightly larger and there is a shallow taper at the top of the hole. This structure makes it easy to insert additional gel boxes from above and provides a good seal at the interface. For the upper gel boxes, the glass tube protrudes 2 cm above the hole, providing a large length of tube above the top of where the gel will be set for loading samples. Gels can be polymerized in the glass tubes over night. Prior to stacking multiple gel boxes together, the space above each gel (in the hole on top of the lower gel boxes) is filled with run buffer to prevent air bubbles from being trapped between the gel columns. The upper gel box is also filled with run buffer, leakage being prevented at the “ducking” interface because the cross-linked polymer gels seal the glass tubes. Protein leakage is also prevented since the interface is electrically floated, therefore, the electrical field remains confined within the tube and there is no other force to drive protein radially. After the initial electrophoresis run and subsequent un-stacking of the gel boxes, the gaps above the lower gel columns are filled with run buffer again and the electric field is turned on for 30-60 seconds to drive protein molecules remaining in solution into the lower gel column. Finally, the buffer containers for each separated box are filled up and electrophoresis is continued. The glass capillary tubes in the elution unit base plate are attached to a holder, under which a motorized fraction collector using standard 9 mm well spacing 96-well plates is located.

In other embodiments, the elution unit, gel segment boxes and gel column tube can be made of any polymer or glass material that is inert and will not react to the electrophoretic current. In a preferred embodiment, the elution unit and gel segment boxes comprise Lucite or acrylic material and the gel column tubes comprise glass. In other embodiments, the electrode is any metal that can be used as a conducting electrode including platinum,

The present multi-channel system further comprises a power supply, and manual or digital control of the power supply and electrophoresis conditions. Typical electrophoresis condition is 20-30 volts/cm, with a power limit of 1-2 watts/column. Power supplies for electrophoresis applications can be obtained commercially such as the VWR® Power Supply Model 202 (VWR Catalog #93000-746; VWR, West Chester, Pa.) which has four sets of color-coded output terminals allow multiple gels to be run simultaneously.

A motorized fraction collector using standard 96-well plates is located below the elution unit. The distance between the capillary tube and the fraction collector can vary. In one embodiment, the fraction collectors are about 15-20 mm below the capillary tube. In one embodiment, there are two identical but individually addressable fraction collectors, each of which supports two 4-channel electrophoresis units. The fraction collector can be on an XY stage that is pneumatically or digitally controlled.

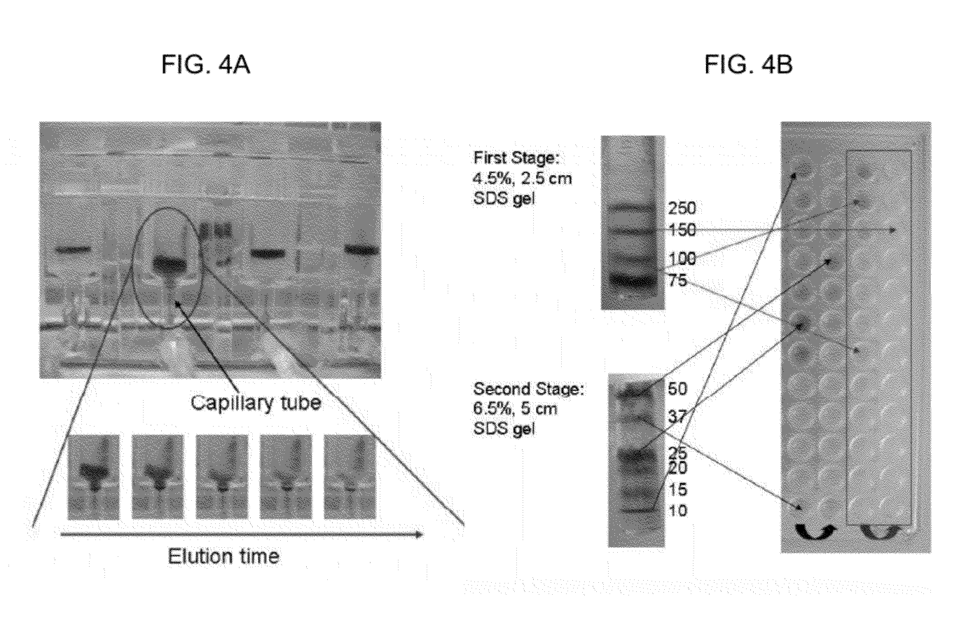

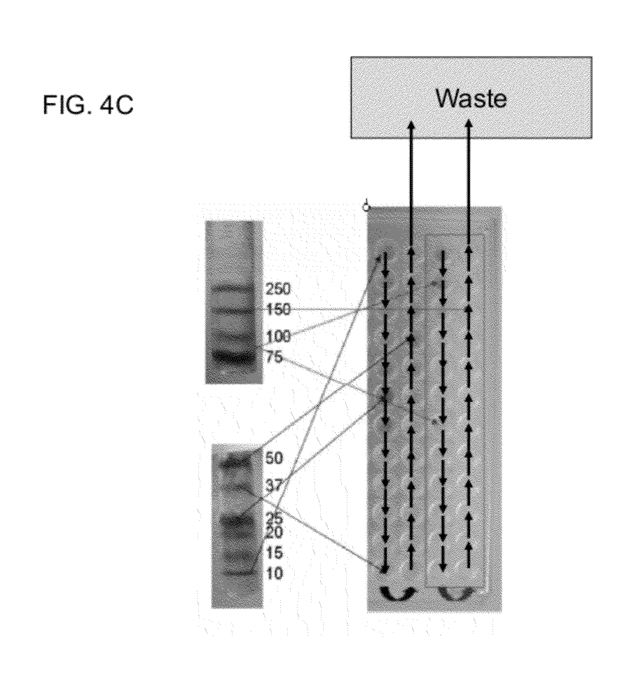

Referring now to FIG. 3 and FIG. 4C, the fraction collectors are an X stage with a pneumatic control on the Y-axis. Four samples from a four-channel system are acquired at a time so 12×2 samples are taken. When the operator is ready to start acquiring samples they “Start” the sequence. FIG. 4C shows in arrows the sequence for two of the four tips of a four-channel system. Since all four tips drip into the plate at the same time the sequence for each of them is the same. The movement of the plate up and down under the tips is accomplished using a motor. The Left right motion is done with a pneumatic actuator. The software control can be based on the following steps shown in Table 1 if the fraction collection plate has 12×8 wells.

TABLE 1
Actuation Controls for Fractionation Collectors

| Action | Object | Value | Description |
|---|---|---|---|
| Output | Move 1 | Left | Move the plate so that the samples drop |
| | | | into the “Left” wells |
| Move To | 1 | 0 | Move the motor to the first of 12 rows |
| | | | on the plate |
| Wait (sec) | | 90 | Wait 90 seconds |
| Loop | | 11 | Loop over the next 11 rows of the plate |
| Move By | 1 | 1 | Move the motor so the plate position |
| | | | changes by one well |
| Wait (sec) | | 90 | Wait 90 seconds |
| End | | | End Loop |
| Output | Move 1 | Right | Move the plate so that the samples |
| | | | drop into the “Right” wells |
| Wait (sec) | | 90 | Wait 90 seconds |
| Loop | | 11 | Loop over the next 11 rows of the plate |
| Move By | 1 | −1 | Move the motor so the plate position |
| | | | changes by minus one well |
| Wait (sec) | | 90 | Wait 90 seconds |
| End | | | End Loop |
| Move To | 1 | −5 | Move to waste location |

Computer software was made using is C# code on a Windows computer to control the “Y” stage pneumatic actuator that toggles between a “Left” and a “Right” position. There were two separate acquisition setups that could be controlled. The “Object”s are “Move 1” and motor “1” for setup one and “Move 2” and motor “2” for setup 2.

As shown in FIGS. 2B and 2C, the elution unit can further comprise a vacuum and/or back pressure valve to control the flow rate. These valves can be controlled manually, pneumatically or digitally.

Modes of operation. The instrument can be operated in two different modes. In the “single” mode, the sample is loaded above a single-piece, long gel column, and the proteins are separated and eluted directly into the fraction collector. Since there is only one segment of gel used in this mode, it can achieve effective separation over a finite mass range only, the range being chiefly determined by the concentration of polyacrylamide used. In the “multi” mode, two or three segments of gel columns, each with a different gel concentration, are stacked on top of one another during the initial electrophoresis run, the gels of lower concentration being placed above those of higher concentration. After the faster migrating protein bands have entered the lower gel segment, the upper segments are removed and put onto other elution units where the electrophoresis of slower migrating proteins is completed and the separated bands eluted and collected. The single mode is fast and effective if the application targets a specific band. The single mode can also be used where multiple gel columns of different percent acrylamide target a broader band range of polypeptides in cases where sample consumption is less of an issue. The multi mode is more efficient in separation and sample use but needs more control and parameter optimization in operation. For example, the condition and time of when to separate the two stacked gel columns must be empirically determined. The multi mode set-up functions similarly to that of a traditional analytical mode for protein separation.

Gel Preparation and Electrophoresis. In one embodiment, SDS or native PAGE gels can be cast in the gel column tubes. In one embodiment, the bottom of the glass tube column is sealed using thin plastic film, then gel solution is poured into the column to the desired length and section of water-saturated solvent is added on top to separate gel solution from air and to maintain a flat gel surface and polymerized. It is preferred that the gel solution is made fresh each time. A typical gel solution that can be used is solutions of 30% acrylamide (Bio-Rad, Cat. #161-0156), 375 mM TrisHCl buffer (pH 7.8), 0.1% (v/v) TEMED and 0.03% (v/v) APS.

When using the gel columns in stacking mode, upon solidification of the gel columns, a stacking gel solution is added. The amount of stacking gel solution used depends on the desired length and butanol added again. Rinse gel surface with Millipore water once the gel is solidified to get ride of excess butanol. Electrophoresis running and loading buffer is added to the glass tubes and multi-channel system prior to an electrophoresis run. If multiple gel segment containers are used, then running buffer needs only to be added to the top-most container and the elution unit as buffer will flow through the gel column tube from the top container, thus also completing the electrophoresis circuit.

The maximum field that could be applied for electrophoresis was 30 volts/cm. Going beyond this threshold resulted in local over-heating, causing gas bubbles to form between the bottom of the gel column and the taper, which interrupts electrophoresis.

Our current Counter Free-Flow PAGE protocols could be further optimized for specific needs. To improve resolution of low-molecular weight proteins or peptides, one could use higher acrylamide concentrations and/or longer gels, increase the counter-flow rate, and collect smaller fractions. To reduce dilution in the high molecular range, one might use lower acrylamide concentrations in the upper gel segment in the multi mode and increase the electric field. However, there are limits to the optimization of certain variables. For example, separation properties will become less reproducible as acrylamide concentrations approach 3% or less. Similarly, higher electric fields are more likely to generate air bubbles at the interface between the gel and the conduit, which will change the electrical field, sometimes completely interrupting electrophoresis

The eluted fractions are delivered directly into multiwell plates, where the molecules can be digested, if necessary, and directly analyzed by mass spectrometry or other techniques. The method can be used in native (or denatured) protein electrophoresis to analyze protein complexes in biological systems. In one embodiment, the fractions collected from the elution units can be further processed by other chromatography means such as Hydrophobic Interaction Chromatography (HIC), Size Exclusion chromatography, Hydrophobic interaction separation, or Chromatofocusing.

Sample Preparation. Crude DNA extracts (˜5 mg/ml, typical) are prepared with an addition sample volume of sample loading buffer, e.g., 60 mM Tris, 460.8 mM, 60% glycerol, 0.03% Bromophenol Blue. For SDS samples, the loading buffer may contain additional SDS and samples were denatured at 95 degree C. for 10 minutes prior to sample loading. For sample with high salt, such as HIC fractions, a desalt column can be used to remove salt and buffer exchanged into 125 mM TrisHCl pH 6.8 buffer. To concentrate desalt HIC samples, an HIC column such as Millipore's 3K Amicon Ultra column (UFC800324) can be used, reducing sample volume to 200˜600 μl per fraction.

Capturing Eluted Proteins. Multiple geometrical and dynamic parameters can affect the overall collection efficiency and ultimate resolution of this device; but it was found that the most critical ones were flow rate of the elution buffer and the relative location of capillary tube in the conduit.

As described above, a fraction collection stage with fraction collector containers or multi-well plates is used for capturing protein bands migrating off the gel column. The user can determine how many fractions are needed to capture and how much fluid volume should be collected in each fraction. For example, in the present embodiment, about 120 μL are captured in each fraction.

In one embodiment, given the structure and geometry of the current collection device, the optimal flow rate is set to about 125 μl/minute and the position of the capillary set to 2 mm below the tapered section. The flow rate and the position of the capillary set are empirically determined by monitoring the collection process of the band of the loading dye. The dye molecules used in the sample loading buffer form a blue band (1˜2 mm wide) that always emerges first from the bottom of gel column. When the elution parameters are less optimal, dye molecules will bypass the tip of capillary tube, and eventually leak out from the conducting holes. With optimal settings, leakage is minimal or not visible (see FIG. 4A). Loading an excess amount of dye molecules and speeding up their migration in the gel, however, does result in more leakage. In general, the leakage decreased as the flow rate increased, and the trade-off was the dilution of proteins in the collected fractions. In another experiment where the capillary was placed at a slightly less optimal location, the total proteins eluted into 48 fractions after native gel electrophoresis was measured and found it was nearly 50% of total the protein loaded (data not shown).

This fraction collection technique does not concentrate separated bands as some other application might require. In one sample process, proteins in fractions selected for mass spectrometry analysis will be captured by the PVDF membrane of a 96-well plate (for example, MultiScreen-HV, Cat. MAHVN4510, Millipore Co. (Bellerica, Mass.)) format and digested thereafter. Therefore, a slight sample dilution is tolerable. In fact, we could even increase flow rate to capture more eluted molecules, resulting in more dilution but still having no impact on our mass spectrometry analysis. A true advantage of the current version of counter free-flow approach is that it is quite easy to construct and to operate in a multi-channel format.

For an application where sample dilution is critical, the coaxial sheath flow (Muller, O.; Foret, F; Karger, B. Anal. Chem. 1995, 67, 2974-2980) and the sweeping approach (Hjerten, S.; Zhu, M.-D. J. Chromatogr. 1985, 327, 157-164) would be advantageous. In fact, we have investigated applying these approaches in our system, but the results were less satisfactory. The main problem was that significantly more engineering efforts, in the areas of design architecture, precision machining and fabrication as well as controls, for example, must be made, especially considering the size of the gel column (not the typical capillary tube) used in this work. It should be pointed out that if a smaller size gel column is used in applications where much less sample is involved, we can simply reduce the size of the upper cup or employ an adaptor and decrease flow rate to accommodate.

Regarding the “stacking” mode of operation, to obtain reproducible separation results, namely, the same protein to be eluted into same or neighboring fractions from run to run, much QA/QC work is needed. For example, the gels must be cast with highly reproducible quality and run in same speed in order to allow reproducible separation between the two gel pieces. These require locking-down all critical operation parameters, which involves a large amount of testing and evaluation. For this reason, we are running the system more frequently in “single” mode at present.

The entire system can be set-up for conducting electrophoresis and fraction collection in various settings including the laboratory or a cold room if sample preservation is a concern. Alternatively, other controls or attachments for temperature control are contemplated to be added by one having skill in the art.

Choice of iTRAQ-Based MALDI MS/MS for Protein Elution Profiling

A major challenge in establishing the feasibility of our proposed tagless strategy was to select a suitable mass spectrometry method. Because of the large number of fractions to be analyzed it was critical to adopt an approach that minimized the number of MS/MS analyses as this could otherwise become a serious rate limiting step. It was also essential to adopt a method that was able to quantitate relative abundances of polypeptides in different fractions.

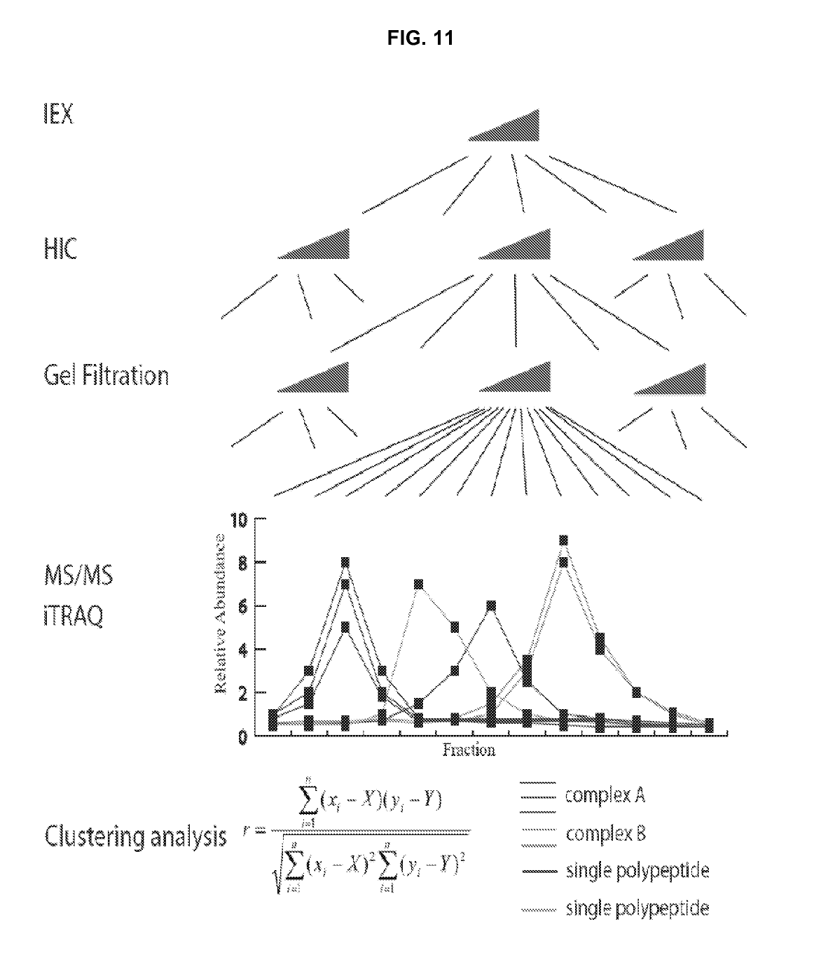

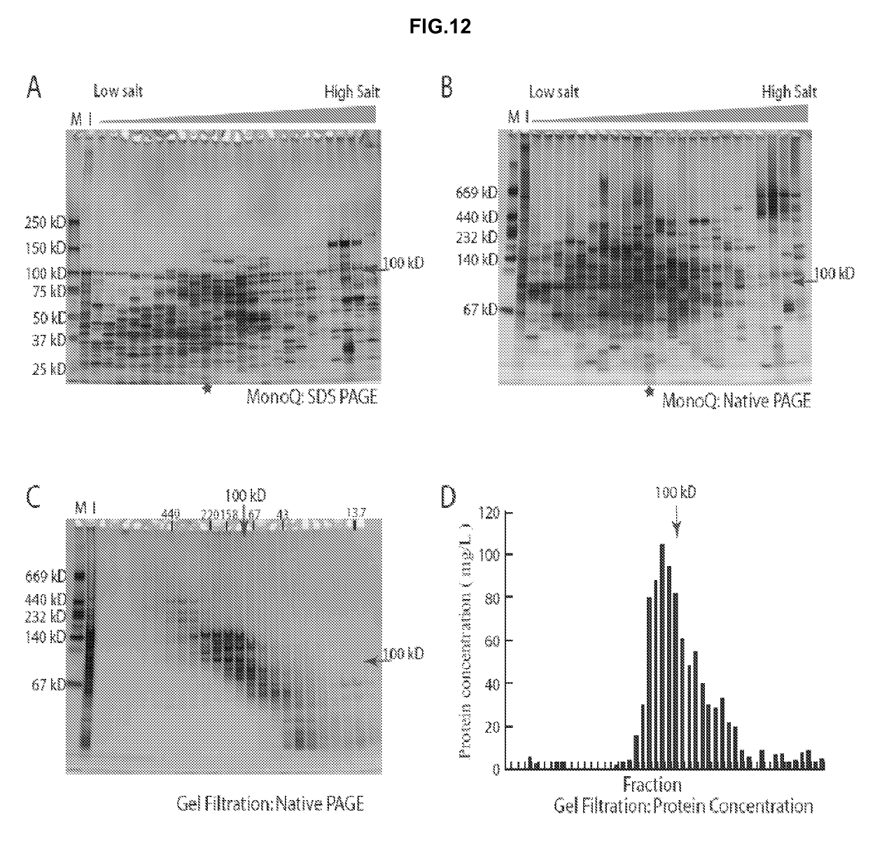

The inventors chose a LC MALDI MS workflow, rather than a LC electrospray ionization (ESI) workflow, as this decouples the LC step from the MS and MS/MS steps, and thus allows repeated interrogation of archived MALDI sample plates. In the context of our proposed analysis of a series of closely related fractions of similar content (FIG. 11), information generated in the course of MS/MS runs on preceding fractions can then be used to design more efficient MS/MS data acquisition strategies for the fractions that follow thus reducing the overall time of MS/MS analysis.

To track changing relative abundances of polypeptides between fractions, the inventors chose an isotopic dilution method that employs the primary amine-directed iTRAQ reagent as the label.^{17, 23} The iTRAQ labeling methodology is the most robust high throughput means of quantifying protein relative abundances by MALDI TOF MS/MS and offers an accuracy and precision comparable with the label-free ESI-based²⁴⁻²⁷ and MALDI-based²⁸ methods. Furthermore, unlike other potential mass spectrometry labeling methods,²⁹ iTRAQ multiplexes four samples in one analysis, further reducing the number of MS/MS analyses required.

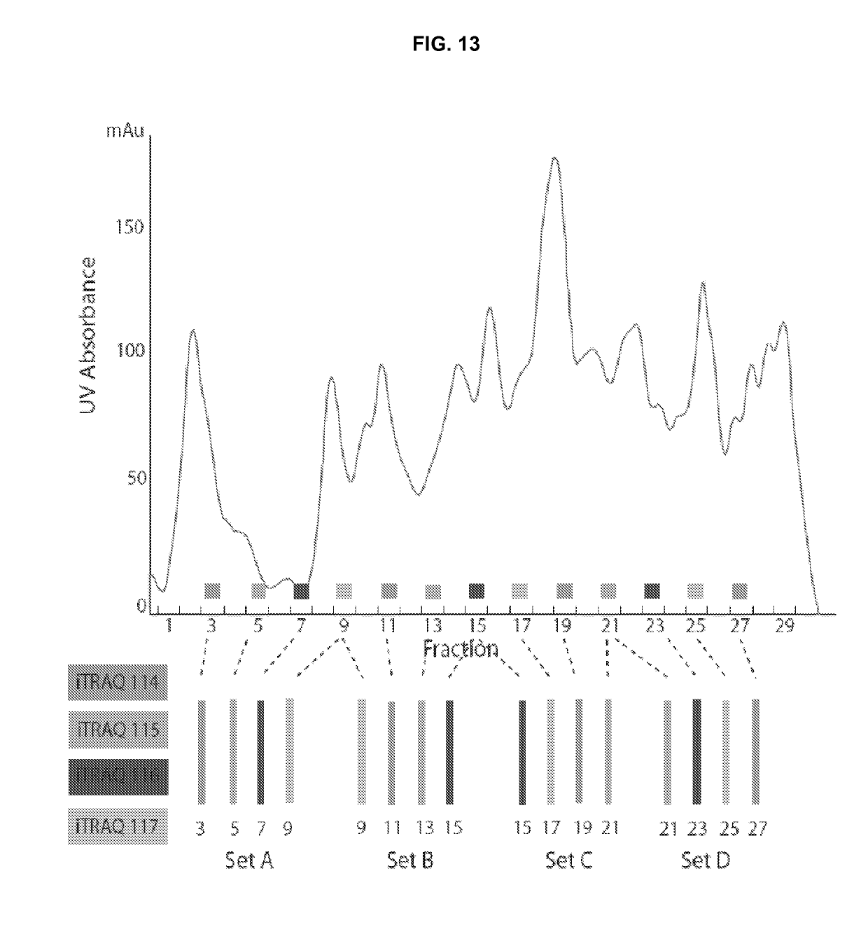

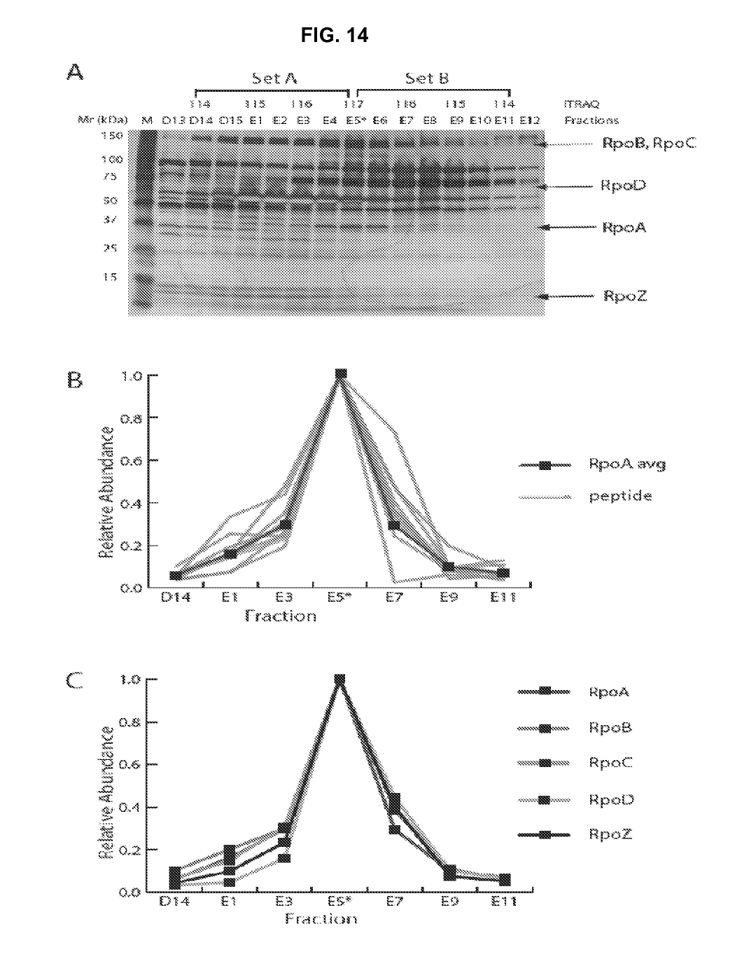

The iTRAQ reagent was originally developed for comparing relative levels of peptides in protein expression profiling experiments. The inventors have adapted it in the following way for our purposes (FIG. 13). A subset of fractions from a single chromatographic separation step is analyzed at a frequency based on the resolution of the chromatography method. For example, every other fraction across a column may be selected. Small aliquots from these fractions are then digested with trypsin and labeled with one of the four different iTRAQ reagents such that each fraction within a multiplex set is represented by one of the four iTRAQ reporter ions at m/z of 114, 115, 116 or 117. Each set contains four sequential fractions from the protein column elute. Every pair of adjacent multiplexes shares a border fraction and hence, all multiplexes representing a single protein separation step are strung together via a series of shared fractions (FIG. 13). LC MALDI TOF MS/MS analysis of each multiplex set produces sequence-specific gas phase product ions from which a peptide is matched to its parent polypeptide. Concurrently, the parent polypeptide's relative abundance, within the analyzed fraction set, is calculated based on the intensities of four iTRAQ reporter ions. A similar approach of using either iCAT™ or iTRAQ reagent-labeling to follow protein gradient distribution profiles under conditions of sedimentation was recently introduced by Kathryn Lilley et al.³⁰⁻³²

Reproducibility of iTRAQ-Based Protein Elution Profiling

In spite of this encouraging result, if a full scale implementation of the tagless strategy is to be successful, iTRAQ-based quantitation and column chromatography will have to be sufficiently reproducible so that data from different fractions, multiplex sets, columns, and days can be compared as part of a large single dataset. Therefore, reproducibility of the tagless method was examined at three levels: (a) reproducibility of mass spectrometric data acquired on a single instrument with the same spotted samples; (b) reproducibility of tryptic digestion, labeling and other sample preparation steps; (c) reproducibility of replica chromatography separations of protein mixtures.

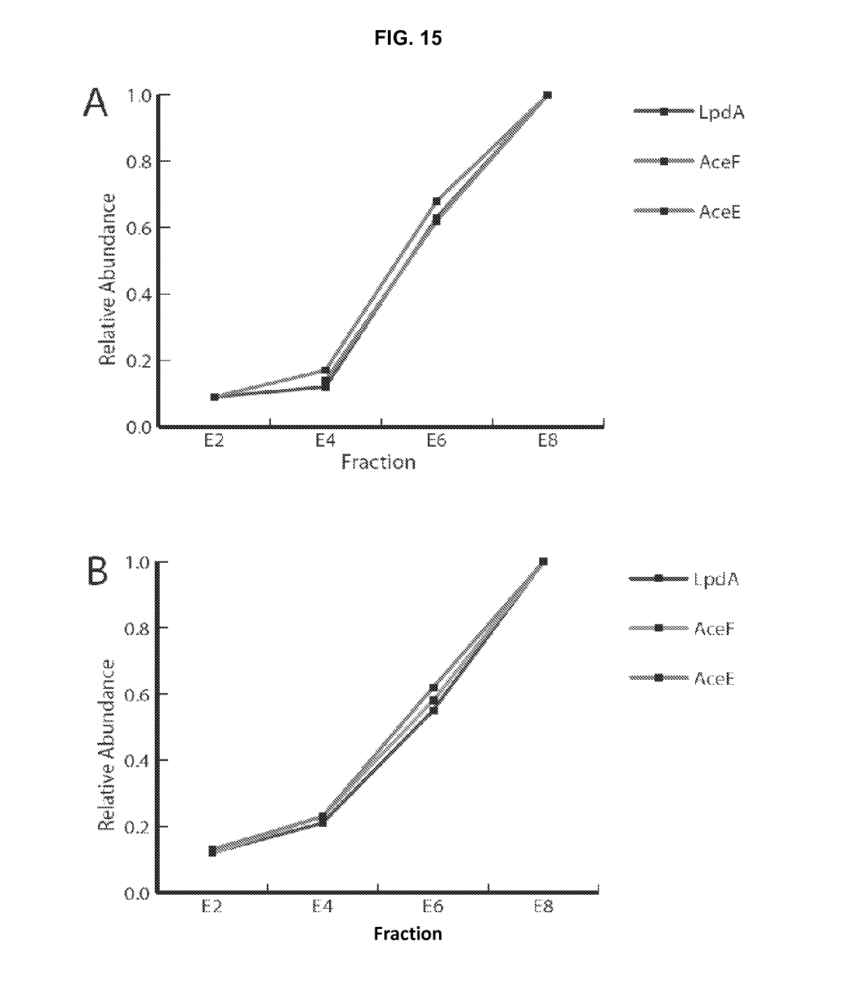

Repeated analysis of the same LC MALDI plate gave essentially the same iTRAQ ratio values for relative abundances of polypeptides, indicating high analytical reproducibly of the mass spectrometers employed in this study (data not shown). Duplicate proteolytic digestion and iTRAQ reagent-labeling performed on the same set of fractions and followed by separate LC MALDI MS/MS analysis produced very similar elution profiles for components of the pyruvate dehydrogenase complex. In both experiments, subunits AceE, AceF and LpdA had similar elution profiles between fractions E4 and E8, suggesting that sample preparation was fairly reproducible (FIG. 15).

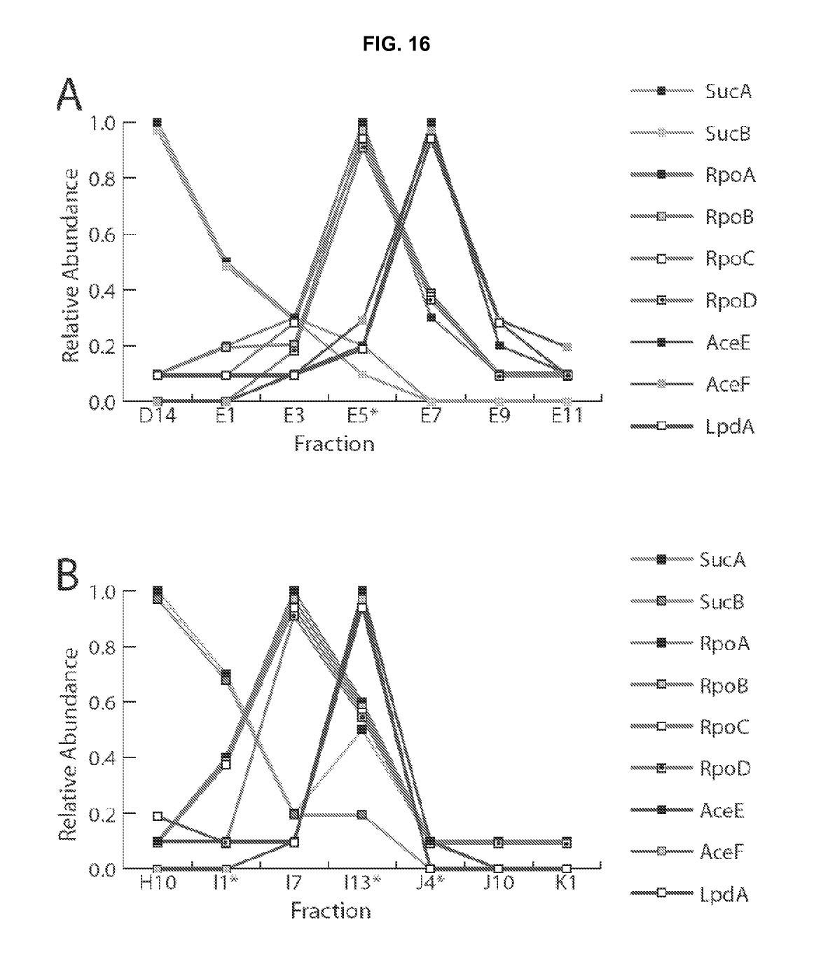

To establish the reproducibility of chromatographic separations, two protein fractionation experiments were compared. Both used gel filtration followed by anion exchange chromatography of an E. coli lysate but were carried out at different scales using differing amounts of crude extract and different size columns. Despite these significant differences, the RNA polymerase, pyruvate dehydrogenase and 2-oxoglutarate dehydrogenase complexes all eluted in the same order and maintained very similar elution patterns (FIG. 16 panel A and B). Thus this result demonstrated that it should be possible to compare protein elution profiles between parallel columns at the same step of a tagless fractionation of a single extract or between equivalent columns in different tagless fractionations of extracts derived, for instance, from cells grown under dissimilar conditions to detect differences in protein complex composition.

Identifying known protein complexes. Next it was necessary to more thoroughly test the feasibility of identifying protein complexes by tracking polypeptide elution profiles using the iTRAQ approach by examining profiles of members of known protein complexes. This was accomplished by assaying 15 fractions grouped into five linked multiplex sets from across the larger scale Mono Q chromatography fractions described above. All fractions were initially analyzed utilizing 4700 Proteomics Analyzer (Applied Biosystems) and then the first 10 fractions encompassed by three four-plexes were re-spotted and re-analyzed using 4800 Proteomics Analyzer (Applied Biosystems). A total of 103 non-ribosomal polypeptides were identified on the basis of at least one peptide (Table 3). Then the literature was consulted to learn how many known protein complexes and protein-protein interactions were to be expected among the polypeptides that were detected. The inventors ignored the fact that for some complexes, usually lower abundance ones, only a subset of polypeptides were identified and instead focused on whether those polypeptides that the inventors could detect were identifiable as co-migrating in the iTRAQ data. According to the EcoCyc database (found online at the BIOCYC website), 35 of the polypeptides the inventors detected with the tagless strategy were constituents of 13 known protein complexes, comprising 37 components (Table 2). of 13 known protein complexes, comprising 37 components (Table 2).

TABLE 2
Components of Known E. coli Complexes Identified by the Tagless Strategy

| Complex | Polypeptide | Polypeptide | Uniprot | | Polypeptide | Sequence | Clustered^{d} | Apex shared^{e} | Co-elution^{f} |
|---|---|---|---|---|---|---|---|---|---|
| ID^{a} | ID# | code name | accession# | ID category^{b} | MW (Da) | coverage (%)^{c} | Yes or No | Yes or No | Yes or No |
| A | 37 | GyrA | P0AES4 | [ID2+] | 96,964 | 25.4 | N | | |
| A | 38 | GyrB | P0AES6 | [ID2+] | 89,950 | 5.1 | N | | |
| B | 54 | Lpp | P69776 | [ID1] | 8,323 | 15.4 | Y | Y | Y |
| B | 62 | PaL | P0A912 | [ID2+] | 18,824 | 21.4 | Y | Y | Y |
| B | 91 | TolB | P0A855 | [ID2+] | 45,956 | 4.4 | Y | Y | Y |
| C | 15 | DnaJ | P08622 | [ID1] | 41,044 | 2.7 | N | Y | Y |
| C | 16 | DnaK | P0A6Y8 | [ID2+] | 69,115 | 37.0 | N | Y | Y |
| C | 34 | GrpE | P09372 | [ID2+] | 21,798 | 25.4 | N | Y | Y |
| D | 6 | AtpA | P0ABB0 | [ID2+] | 55,222 | 1.8 | Y | Y | Y |
| D | 7 | AtpD | P0ABB4 | [ID2+] | 50,325 | 6.5 | Y | Y | Y |
| E | 14 | DnaE | P10443 | [ID2+] | 129,905 | 1.2 | Y | Y | Y |
| E | 17 | DnaX | P06710 | [ID2+] | 71,138 | 4.2 | Y | Y | Y |
| E | 43 | HolE | P0ABS9 | [ID1] | 8,846 | 9.2 | Y | Y | Y |
| F | 11 | CysI | P17846 | [ID2+] | 63,998 | 4.2 | N | Y | Y |
| F | 12 | CysJ | P38038 | [ID2+] | 66,270 | 8.5 | N | Y | Y |
| G | 61 | NusA | P0AFF6 | [ID2+] | 54,871 | 16.8 | Y | Y | Y |
| G | 75 | Rho | P0AG30 | [ID2+] | 47,004 | 18.6 | N | | Y |
| G | 79 | RpoA | P0A7Z4 | [ID2+] | 36,512 | 27.7 | Y | Y | Y |
| G | 80 | RpoB | P0A8V2 | [ID2+] | 150,632 | 25.3 | Y | Y | Y |
| G | 81 | RpoC | P0A8T7 | [ID2+] | 155,160 | 28.4 | Y | Y | Y |
| G | 82 | RpoD | P00579 | [ID2+] | 70,263 | 6.2 | Y | Y | Y |
| G | 83 | RpoZ | P0A800 | [ID2+] | 10,237 | 44.0 | N | | Y |
| H | 58 | MukB | P22523 | [ID2+] | 170,230 | 25.2 | N | Y | Y |
| H | 59 | MukE | P22524 | [ID2+] | 28,178 | 8.6 | Y | Y | Y |
| H | 60 | MukF | P60293 | [ID2+] | 50,597 | 9.3 | Y | Y | Y |
| I | 1 | AceE | P0AFG8 | [ID2+] | 99,668 | 44.2 | Y | Y | Y |
| I | 2 | AceF | P06959 | [ID2+] | 66,096 | 47.8 | Y | Y | Y |
| I | 53 | LpdA | P0A9P0 | [ID2+] | 50,688 | 38.0 | N | Y | Y |
| J | 87 | SucA | P0AFG3 | [ID2+] | 105,062 | 25.2 | N | Y | Y |
| J | 88 | SucB | P07016 | [ID2+] | 44,011 | 39.5 | Y | Y | Y |
| J | 53 | LpdA | P0A9P0 | [ID2+] | 50,688 | 38.0 | Y | Y | Y |
| K | 25 | GcvP | P33195 | [ID1] | 104,376 | 0.9 | N | | Y |
| K | 53 | LpdA | P0A9P0 | [ID2+] | 50,688 | 38.0 | N | | Y |
| L | 72 | PyrB | P0A786 | [ID1] | 34,427 | 3.2 | Y | Y | Y |
| L | 73 | PyrI | P0A7F3 | [ID1] | 17,121 | 6.5 | Y | Y | Y |
| M | 31 | GlyQ | P00960 | [ID1] | 44,716 | 3.3 | N | | Y |
| M | 32 | GlyS | P00961 | [ID2+] | 76,813 | 23.4 | N | | Y |

^{a}Protein complex data based upon the content of the Encyclopedia of E. coli K-12 Genes and Metabolism (http://biocyc.org/ECOLI/new-image?object=Protein-Complexes).
^{b}Protein identification based on one and two or more peptides for [ID1] and [ID2+] categories, respectively. MS/MS spectra of polypeptides matched to a single peptide are shown in Supplemental FIG. 3.
^{c}Unique non-overlapping peptides were used to calculate protein sequence coverage defined as the ratio between the sum of amino acids encompassed by the confidently matched peptides (% CI > 95%) and the number of amino acids in a polypeptide sequence. For polypeptides observed in more than one four-plex, the best four-plex data are shown.
^{d}Elution profiles of all detected non-ribosomal proteins were compared using a modified Pearson's algorithm. “Y” means that at least two complex components were clustered at a threshod of 0.92.
^{e}At least two complex components shared at least one apex of elution.
^{f}At least two complex components eluted in the same fraction.

According to a large scale TAP analysis of protein-protein interactions in E. coli¹⁵, 21 of the polypeptides the inventors detected with the tagless strategy were expected to participate in 24 reciprocal pair-wise interactions (Table 4). There was a significant overlap between these two sets of polypeptides since many of the components of known protein complexes were also detected by TAP methodology.

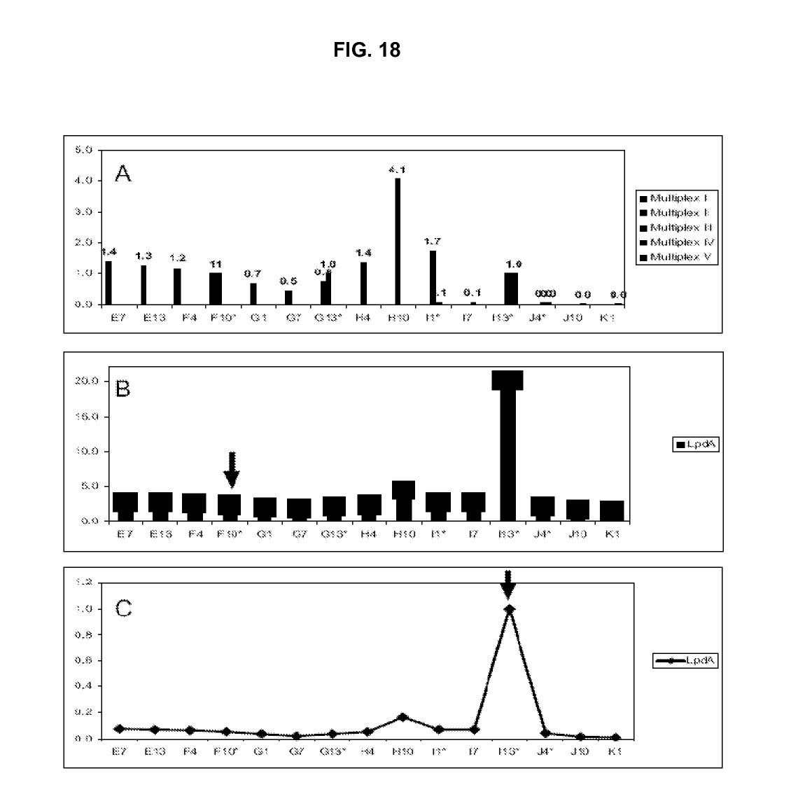

An ad hoc approach was employed to classify co-migrating polypeptides in the iTRAQ data based on polypeptides that showed maximum concentrations in the same fraction (elution apices). Of the known complex components from the EcoCyc database, the great majority (78%) shared the same elution apex. An additional 16% were detected in the same fractions, and hence 95% of the expected protein complex components demonstrated close co-elution (FIG. 18). Only DNA gyrase components GyrA and GyrB showed completely disparate elution profiles (FIG. 18A), but this is not surprising as DNA gyrase is known to be unstable. ³³ Of the pair wise interactions defined by TAP, 63% of partners verified by reciprocal TAP shared elution apices and additional 25% were found in the same fraction, bringing the detection of closely co-eluting partners to 88%. Thus, the iTRAQ strategy does allow a range of different complexes to be detected.

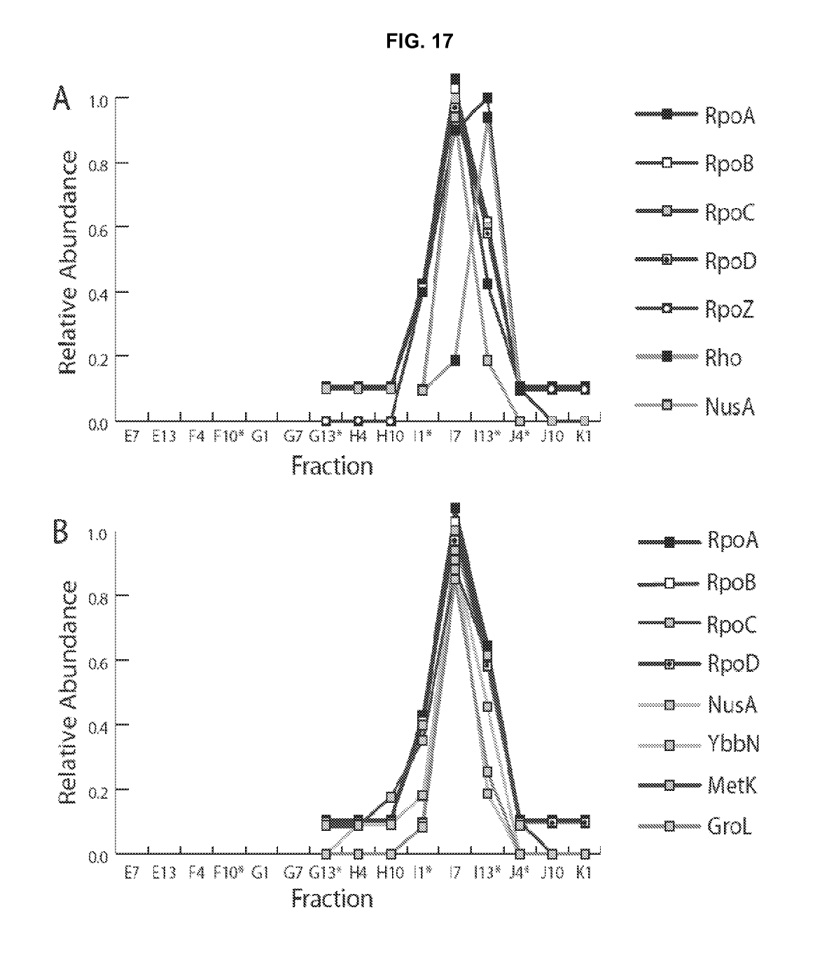

Despite the broad similarity in elution profiles for known complex components, some intriguing differences between their profiles were seen in a few cases. For example, RNA polymerase components NusA and Rho demonstrated much narrower elution peaks than the core RNA polymerase subunits RpoA, B, and C (FIG. 17A), which likely is due to the different chromatographic properties of the known distinct forms of RNA polymerase causing the core subunits that participate in all forms of polymerase to have broad profile while particular NusA and/or Rho containing sub-form(s) instead fractionate more discretely. Thus, even if an agreement between the anticipated and observed elution profiles of known complex components is not complete, it cannot be assumed that this represents an artifact of the tagless methodology or some error in our methods. Rather, an observed discrepancy may reflect biologically relevant differences.

Discovery of complexes by automated cluster analysis. The above analyses used ad hoc criteria to judge if polypeptide elution profiles were sufficiently similar to suggest that they are members of the same protein complex. However, adaptation of the tagless strategy in a high-throughput modality which generates many more fractions will require automated statistical analyses that can identify putative protein complexes and provide confidence estimates on the likelihood of that prediction. Towards accomplishing this goal, a prototype algorithm for automatically detecting complexes based on the clustering methods used, to detect co-regulated genes in expression microarray data³⁴ was tested.

In general, the elution profile of a polypeptide can be plotted as an intensity map in a multi-parameter grid space, where the coordinates of each grid specify a fraction and its intensity indicates the relative abundance of the polypeptide. For example, in a two-step protein complex separation scheme, the map could be plotted exactly like a 3-D geological map representing hills and mountains. The task of finding co-migrating polypeptides is then reduced to co-localizing “hill and mountain” peaks within a grid of the N-dimensional map. From the data analysis point of view, each peak is a subset of registered data points and detecting co-localized peaks can be achieved by performing clustering analysis of the subset over the entire collection of protein elution profiles.

The results of such clustering analysis were compared to the manually curated groupings of co-migrating polypeptides. Our current clustering algorithm correctly grouped 69% and 87% of the polypeptides sharing the same apex of elution that were manually classified as members of either EcoCyc known complexes or reciprocal TAP-defined interactions, respectively (Table 2 and Table 4). The differences between the ad hoc and computational methods of grouping polypeptides reflected differences in the criteria used. The manual evaluation was based solely on shared fraction and shared apex elution, whereas the clustering algorithm also took into account additional features such as peak shape, peak resolution and the presence of multiple apices within a contiguous portion of polypeptide elution profile. These additional constraints resulted in the exclusion of some of the known complex components from certain clusters. For example, the RNA polymerase components RpoA, RpoB, RpoC, RpoD, and NusA were included in the same cluster, but RpoZ and Rho were not (FIG. 17B). Likewise, two components of a chromosome partitioning complex, MukE and MukF, were placed in the same cluster, but another component, MukB, was not because it eluted in a narrower peak (FIG. 18 H). The “failure” to cluster all complex components as one entity might actually provide important insights into the previously discussed heterogeneity between distinctly eluting “sub-complexes”. Therefore, the future development of the clustering algorithm will necessitate the incorporation of more sophisticated tools, for example, flexible, multi-tiered stringency scales to allow more robust recognition of potential co-eluting polypeptides while preserving detailed information about chromatographic peak shape and resolution.

The data inputted into the clustering analysis was not limited to members of known complexes, but included all 103 non-ribosomal proteins. Not surprisingly, given the crude and complex nature of the chromatography fractions analyzed, clusters that included members of known complexes frequently contained additional polypeptides that seemed unlikely to be uncharacterized members of these complexes (Table 5). For example, YbbN, MetK and GroEL clustered with members of the RNA polymerase complex (FIG. 17B), but have not been associated with this well-studied complex before. Instead, the inventors suspect that the majority of these additional cluster members resulted from fortuitous co-elution within the Mono Q column chromatogram. Incorporation of additional protein separation steps into a full scale tagless separation scheme should greatly reduce the frequency of such “opportunistic co-eluting”, especially since clustering can then take into account co-migration across not one but three of four dimensions. With much more information available, it should also be possible to better distinguish between potential sub-complex forms and identify novel complexes and complex components.

Purification of Protein Complexes

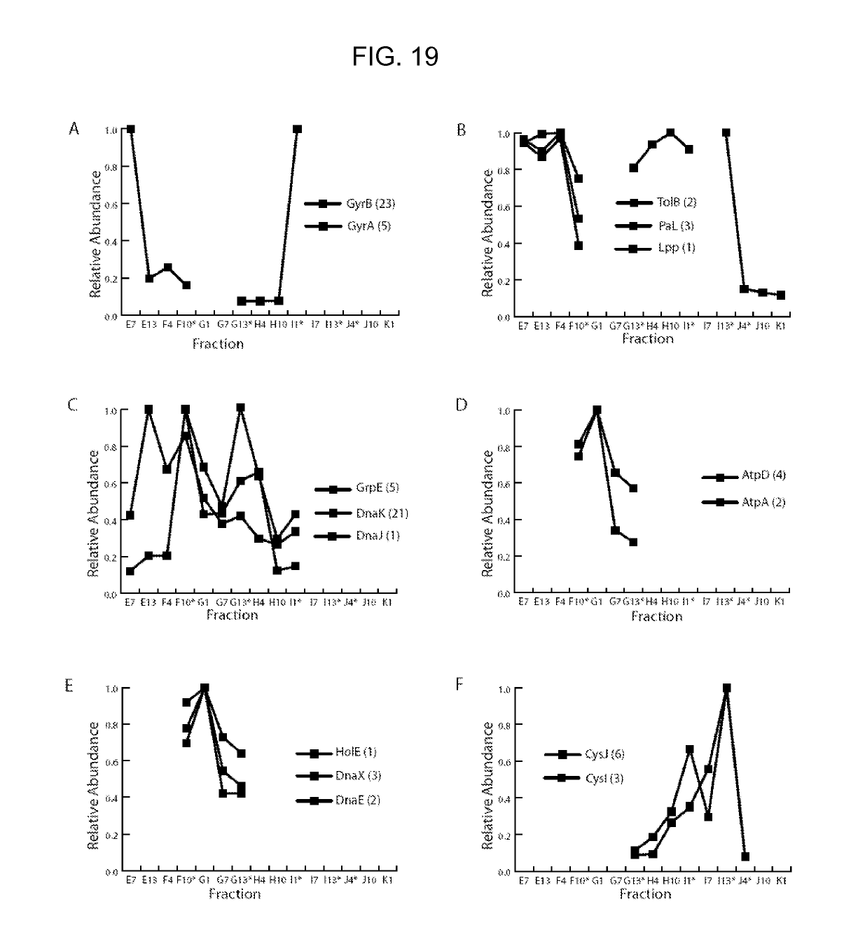

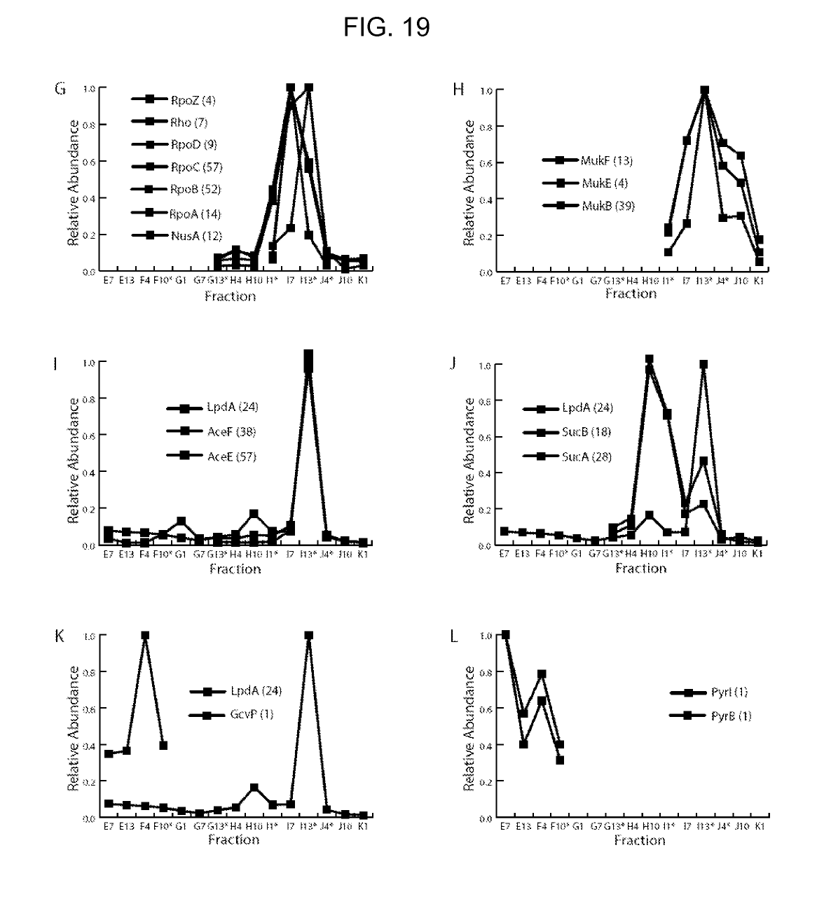

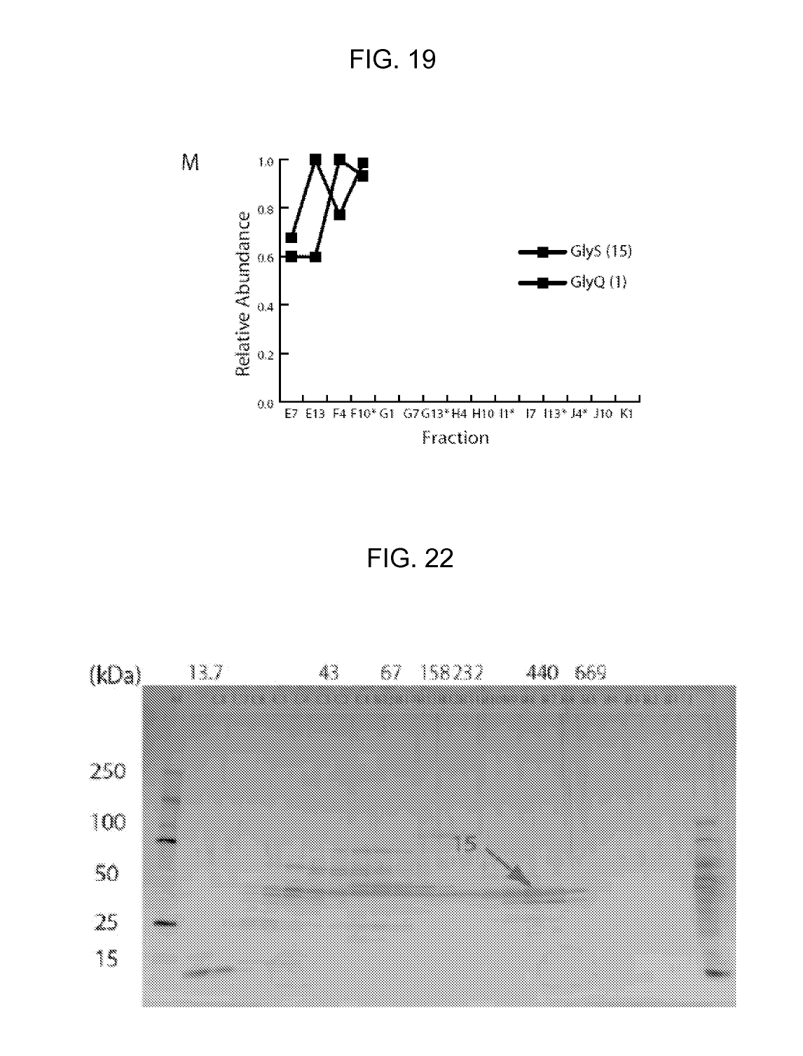

The tagless strategy was able to identify putative protein complexes without a need for complete purification. However, these results indicate that a majority of high and moderately abundant stable protein complexes can be purified to near homogeneity by an optimized tagless fractionation method employing four orthogonal separation steps and scaling up the amount of starting material. Even with the pilot fractionations employed here, i.e., using only anion exchange and size exclusion chromatography, three complexes (pyruvate hydrogenase, RNA polymerase and GroEL) have been purified to apparent homogeneity from E. coli cell lysate (FIG. 19). More extensive fractionation of extracts from the sulfate reducing bacteria D. vulgaris has, to date, purified 3 heteromeric and 25 homomeric complexes from only a small portion of the total fractionation space³⁵. These purified complexes are amenable to further characterization methods such as single particle electron microscopy and Small Angle X-ray Scattering. For example, the 17 Å structure of one complex identified and purified by the tagless scheme has already been obtained.¹⁹

The inventors have established proof of principle evidence for the feasibility of employing a tagless strategy for protein complex identification and purification. The inventors estimate that at least around 50% of bacterial polypeptides participate in complexes that are sufficiently stable to survive the multiple chromatographic steps. The range of complexes identified by the tagless strategy is likely to be comparable to those identified by TAP experiments. Out of 24 TAP-detected reciprocal interactions,¹⁵ only three (MetK-SecA; MetK-DnaJ and GyrA-GyrB) had completely dissociated during purification (Table 4). In addition, there is good reason to believe that those complexes that are disrupted by the use of an affinity tag, and therefore are not detectable by TAP, will be identifiable by a tagless approach. Relative quantification using iTRAQ reagents allowed co-migration of polypeptides to be determined and the chromatographic separation appeared sufficiently reproducible such that results across multiple parallel chromatograph columns, each separating different subsets of total cellular protein, could be meaningfully compared. Even a relatively simple clustering algorithm was effective at automatically detecting members of protein complexes using data from only two dimensions of separation. Several of the more abundant complexes were purified to greater than 70% homogeneity.

The samples analyzed by iTRAQ LC MALDI MS/MS were derived from a subset of the protein fractions of a two dimensional scheme and represented only approximately ten percent, by mass, of the of water soluble E. coli proteins. Thus, even at this current pilot scale it is likely that around one thousand of polypeptides would have been detected had all fractions from the scheme been analyzed by mass spectrometry. The remaining two thousand or so water soluble proteins that would not have been detected are in most cases likely to be of lower abundance. Hence, by starting with large amount of crude protein extract and employing four, rather than two, orthogonal chromatography separation steps, it should be possible to detect the great majority of these lower abundance polypeptides. Of the two constraints inherent to analysis of low abundant species, i.e., dynamic range challenges and availability of material, the former is currently being addressed by performing extensive protein separation involving multiple chromatographic steps. The latter constraint is not a major obstacle since biomass for our target organism D. vulgaris is currently produced on a scale of 400 l scale (4×10¹³-4×10¹⁴ cells) that delivers ˜10 g soluble protein (˜200 μmol of total protein, assuming an average polypeptide MW of 50 kDa). Within this mixture, a low abundance polypeptide expressed at the level of 10 copies per cell will constitute ˜670 pmol material that corresponds to a 3.3×10⁻⁶ portion of total protein. The current yield after the four protein complex separation steps, tryptic digestion and iTRAQ-labeling is estimated at ˜0.5%. Assuming the same level of recovery of low abundance complex components and anticipating a spread of protein complex elution during a 4-step fractionation into 50 fractions, 3.35 pmol of the low abundance protein will be recovered at a level of 67 fmol per fraction or ˜130 fmol per iTRAQ multiplex, assuming the worst case situation when only two fractions within a four-plex might contain a protein complex. This scenario brings us within the current practical detection limits of MALDI TOF/TOF instrument. With expected increase in the sensitivity of mass spectrometers over the next five to ten years, nearly all complexes should be detectable with such a fractionation. The inventors have now established a four dimensional fractionation at this lager scale and are now optimizing each fractionation step (unpublished data). While the success of discovery of any specific low level protein complex will be highly dependent on the extent of its separation from other species, efficiency of digestion and labeling and quality of MS/MS, in principle detection of low abundance complexes is within the realm of possibility.

In another embodiment, the gel apparatus is used to separate the proteins in the tagless survey of proteins. In another embodiment, the apparatus and steps carried out are automated and software-controlled by a computer. A major advantage of the tagless approach is that by its design it is intrinsically more amenable to automation than TAP as it consists of fewer types of operations and is highly repetitious. For example, no genetic manipulation of the organism is required and only one large culture of cells need be grown. With the automation of the sample preparation and chromatographic separations and development of a data analysis pipeline that is coupled to real time control of the mass spectrometer to eliminate redundant and time consuming analysis of peptides from the same protein, and the expected future increase in the speed of MALDI MS/MS instruments, it should be possible to achieve much higher throughput identification of protein complexes than is currently possible.

In other embodiments, additional methods to establish the accuracy and veracity of putative complexes identified by the tagless strategy will be needed. In one embodiment, an increase in the number of fractionation steps and the use of more complex clustering algorithms that employ quantitative data on the migration of polypeptides across four chromatographic dimensions to reduce the occurrences of “opportunistic co-eluting” of unrelated proteins seen in the pilot study. At least in some model organism a subset of putative complexes could and should be verified by reciprocal TAP analysis. In general, it is critical to cross-verify the predictions made by any method to identify protein complexes system wide using a combination of biological and analytical techniques.

In conclusion, the tagless protein complex identification strategy is a discovery as well as a purification tool. Its great strengths lie in the ability to analyze native systems and in the potential of highly automated high throughput execution. The inventors expect that a combination of tagless- and immunoaffinity-based complex isolation strategies will greatly expand the amount of information about the biology of organisms and provide orthogonal confirmation of the overlapping results.

Any figures or details that can not be easily viewed in this patent application can also be found online at the website of a published journal article that relates to the invention. The reference, A “Tagless” Strategy for Identification of Stable Protein Complexes Genome-wide by Multidimensional Orthogonal Chromatographic Separation and iTRAQ Reagent Tracking, J. Proteome Res., 2008, 7 (5), pp 1836-1849, is accordingly fully incorporated by reference in its entirety herein.

### Example 1

### Multi-Channel Electrophoresis System

Based on the scheme described above, a prototype, 16-channel instrument (see FIGS. 2A and 3) has been constructed and tested. It has gel blocks with short (3 cm long) gel columns, which could be used as the lower and middle piece of gel columns, and blocks with longer (5 cm or 12 cm) gel columns as the top and sample loading section. Each block contained a row of four glass tubes (7 mm id), spaced 18 mm center-to-center and glued into an acrylic block as illustrated in FIG. 1, allowing processing of 4 samples at a time. The elution device and the lower buffer container were formed by attaching a base plate holding four capillary tubes and an O-ring gasket to a buffer container body, which included a Pt electrode and buffer inlet and outlet. On the top surface of the buffer container, there were four precisely machined holes for installation of those plastic conduits. The other ends of these glass capillary tubes (300 μm id and 15-25 cm long) were attached to a holder, under which a motorized fraction collector using standard 96-well plates was located. There are two identical but individually addressable fraction collectors; each supports two 4-channel electrophoresis units.

### Example 2

### SDS and Native Page Gels

To cast either SDS or native PAGE gels, first the bottom of the column was sealed using thin plastic film, then gel solution was poured into the column to the desired length and a 5 mm long section of water-saturated butanol was added on top to separate gel solution from air and to maintain a flat gel surface. The gel solution was made fresh each time using stock solutions of 30% acrylamide (Bio-Rad, Cat. #161-0156), 375 mM TrisHCl buffer (pH 7.8), 0.1% (v/v) TEMED and 0.03% (v/v) APS. Upon solidification of separation gel (usually about 3-4 hours), for stacking, 4% stacking gel solution, using 125 mM TrisHCl (pH 6.8) instead, of desire length was poured on top of the gel and butanol added again. The length of stacking gel was kept, at least, twice as the sample's to be loaded, with a minimal of 1 cm. Rinse gel surface with Millipore water once the gel was solidified to get ride of excess butanol. Gels were run in 1× running buffer (10 mM Tris, 76.8 mM Glycine; for SDS gels, add 0.2% (v/v) SDS). Typical electrophoresis condition was 20-30 volts/cm, with a power limit of 1-2 watts/column

### Example 3

### Fraction Collection of Samples

Crude Desulfovibrio vulgaris (D. vulgaris) extracts (˜5 mg/ml, typical), were prepared with an addition ⅓ sample volume of 6× sample loading buffer (60 mM Tris, 460.8 mM, 60% glycerol, 0.03% Bromophenol Blue). For SDS samples, the loading buffer contained additional 18% (v/v) of SDS and samples were denatured at 95 degree C. for 10 minutes prior to sample loading. For sample with high salt, such as the HIC fractions, a desalt column from GE HealthCare (PD-10 Column, Cat. 17-0851-01) was used to remove salt and buffer exchanged into 125 mM TrisHCl pH 6.8 buffer. To concentrate desalt HIC samples, Millipore's 3K Amicon Ultra column (UFC800324) were used, reducing sample volume to 200˜600 μl per fraction. HIC samples were prepared with ⅓ sample volume of 6× Stacking Gel Sample Loading Buffer (375 mM Tris HCl (pH 6.8), 60% (v/v) Glycerol, 0.036% (v/v) Bromophenol Blue.).

To monitor and evaluate separation and elution results of this instrument, fractions were sampled and analyzed by traditional slab technique. 12.5 μl from each fraction sampled was mixed with 3 μl of 6× sample loading buffer (375 mM TrisHCl pH 6.8, 60% glycerol, 0.03% Bromophenol Blue, add 18% of SDS for SDS gel) and loaded onto a slab gel (Bio-Rad's Criterion Tris-HCl Gel, 4-15% (cat. 345-0029) for native samples, 4-20% (cat. 345-0034) for SDS samples). Native gels were run at 200V in 1× Gel Running Buffer (0.01M Tris, 76.8 mM Glycine), SDS gel at 200V in 1× gel running buffer with 0.2% SDS until the dye front reached the bottom of the gel. Gels were stained using Invitrogen's Silver Quest staining kit (LC6070).

### Example 4

### Elution of SDS Page Gels

To demonstrate that this instrument works, we first used it to separate and elute a mixture of denatured proteins by SDS gel electrophoresis. FIG. 4 gives an example of protein bands collected by this instrument. After an initial 32-min run, as shown in two gel columns, 6 bands of pre-stained protein standards (from Bio-Rad Co.) of 50 kD or smaller entered the lower, second gel segment (6.5%) and effectively separated, while the other 4 bands of 75 kD and higher remained in the upper, first gel segment (4.5%). As shown in the scheme shown in FIG. 1D, the top gel segment then removed to another elution device, and completed electrophoresis and elution on both units. From the collected fractions, it is not difficult to see the pattern corresponding to the separated bands in gels.

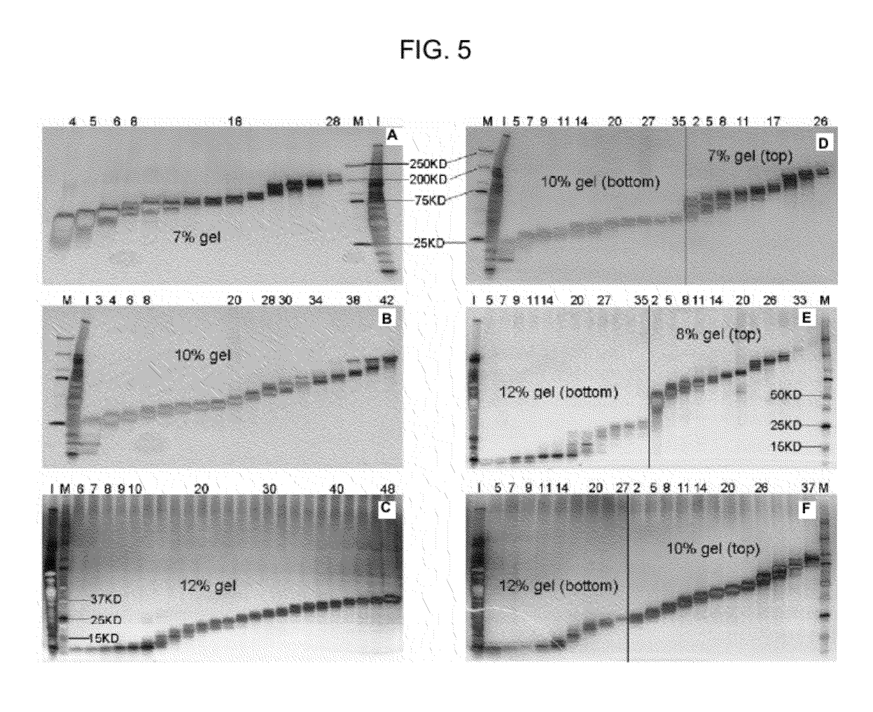

Another test was performed using crude extract of D. vulgaris. FIG. 4 (a, b and c) were obtained using the “direct” mode. Identical samples of crude extract of protein mixture were loaded to 7%, 10% and 12% linear SDS gels. FIG. 5 (d, e and f) were obtained from two-staged (7%+10%, 8%+12%, and 10%+12%) gels in “stacking” mode. A common feature, displayed in all gels, was that each fraction contained only a narrow band of proteins of input mixture and bands of neighboring fractions partially overlap, proving this instrument worked as designed. Another two features were obvious: (1) at the lower mass side, proteins might migrate too fast and be partially resolved only, that is, many bands were eluted into a single fraction; and (2) at higher mass side, the migration might be too slow and a single band could be stretched over many fractions. Clearly, for high speed and high resolution separation, a 2 cm long 12% gel is very good for proteins up to 25 kD, 10% gel is excellent from 25 kD to 100 kD, and 7 or 8% gel is good for 100 kD and up. To cover a broader range, it is necessary to use the “stacking” mode. As demonstrated, excellent results could be obtained by a two-staged 10% and 12% gel from 10 kD to 100 kD (FIG. 5 (f)); a combination of 8% and 12% can extend that range to 150 kD (FIG. 5 (e)). The 7%+10% combination in fig-5 (d) shifted the coverage to 25 kD to 150 kD only. A three-staged gel system might be needed if one wants to cover the entire range (˜a few kD to approximately 200 kD) for denatured proteins. The capacity of this system was relatively high, with good results being obtained when up to 500 μg of protein were loaded per gel column. It should be pointed out that it is necessary to optimize the separation parameters such as gel concentration and length if one wants a most efficient system that targets a specific mass range.

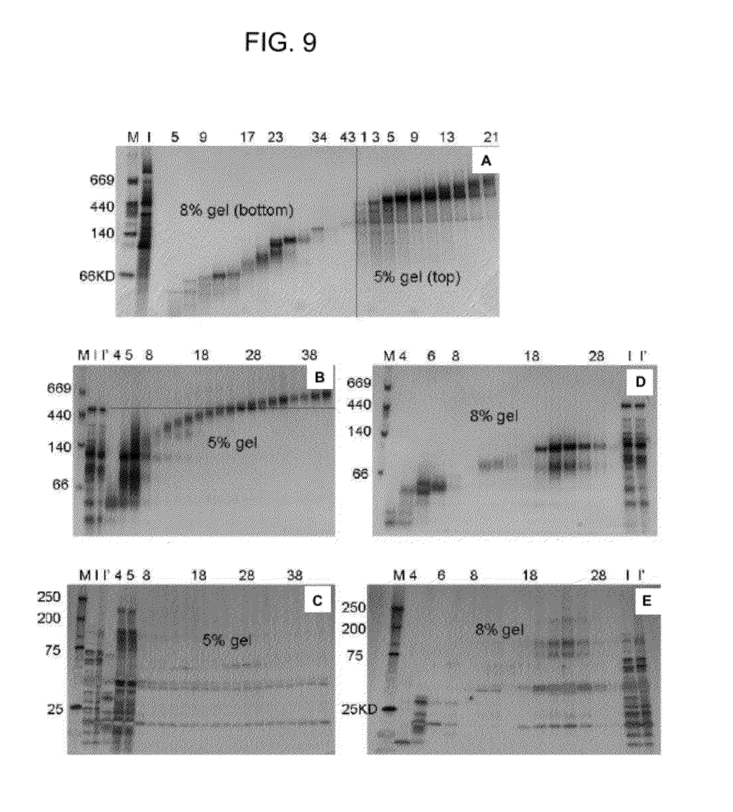

We have found that the protein separation reproducibility of this system is similar to other PAGE instruments, based on approximately 100 test runs performed over 2 years. In each run, the system was disassembled and reassembled with new buffer and gels, and multiple samples (identical replicates or different) were loaded and fractionated. For the same running conditions, the dye front arrival times, from lane-to-lane and day-to-day, typically varied by only 1 to 2 minutes (see FIG. 9) and the position shift of corresponding protein bands eluted and collected was only plus or minus one fraction (FIG. 10).

### Example 5

### Elution of Native Gels

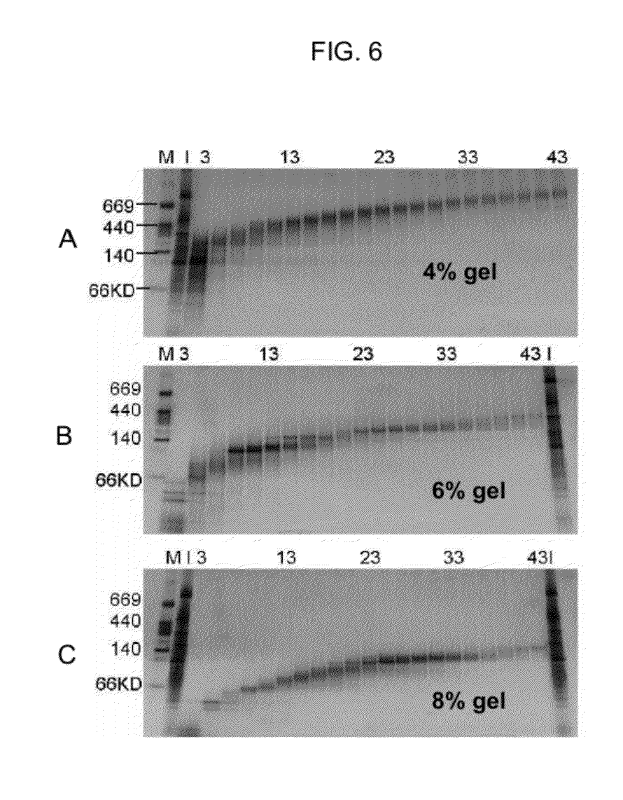

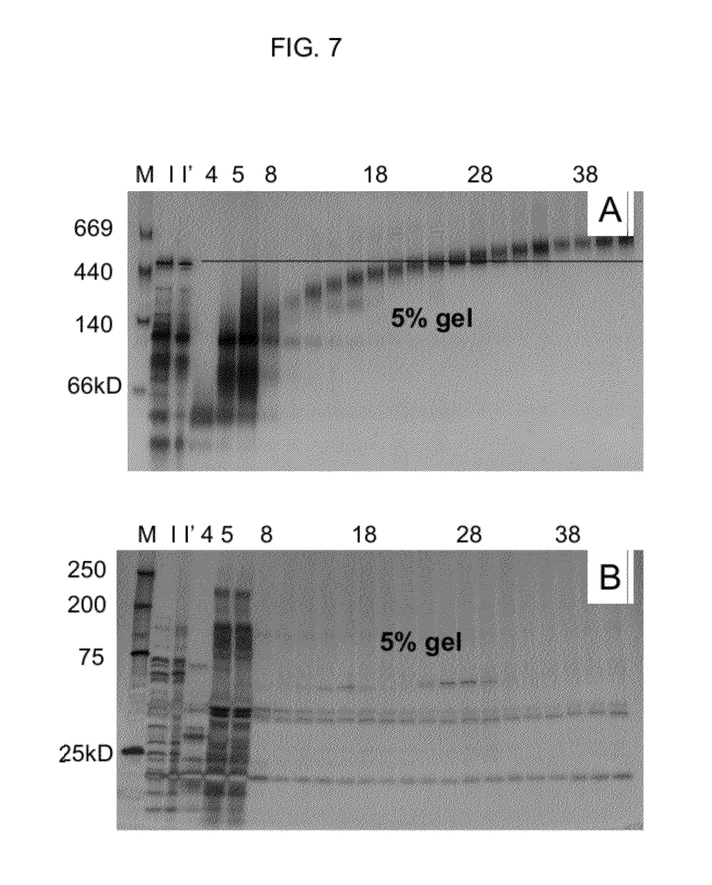

To understand the separation characteristics of linear, native polyacrylamide gels in our free flow electrophoresis system, we first developed protocols for casting and operating different percent gels in the single mode. FIG. 6 shows typical results using 2-cm long gels. The 4% gel provided good separation for protein complexes of masses 400 kD and above, the 6% gel for 150-350 kD, and the 8% gel for 45-180 kD. However, it was also noticed that native gels suffer from what appears to be a protein aggregation problem that reduces their effectiveness, even when the amount of protein loaded is limited to 50 μg. Using a sample that contained only a few large molecular weight protein complexes, a smear of apparently large molecular weight proteins are eluted that do not correspond to any input proteins and many of them are larger than any input protein (see FIG. 7). SDS slab gel analysis of the same eluted protein fractions showed that these “novel” large proteins mostly consisted of small molecular weight polypeptides found in the input sample, and thus appear to be aggregates created during native electrophoresis in the free flow device.

This suggested that a combination of 2 cm long gels of 5% and 8% might just cover the mass range of 30˜450 kD, where most of our proteins and protein complexes were located. FIG. 7 illustrates results of such a combination, including the ones obtained with samples of HIC fractions. In our “tagless” scheme, HIC fractions, all in large volume (2.5 ml per fraction) of high salt buffer, are inputs for native gel electrophoresis. They were desalted and concentrated to 200-600 μl, without apparent loss. This allowed loading of an entire HIC fraction into single gel column.

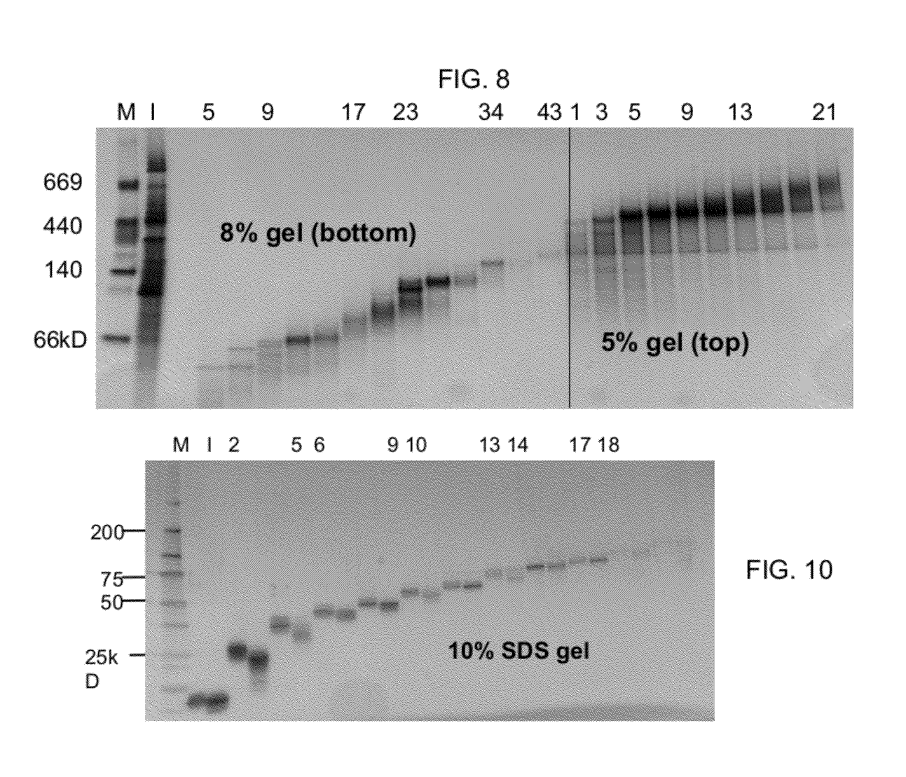

Clearly, the 8% portion worked as anticipated (see FIG. 8 (a and b)) but the 5% appeared to lack resolution in the 200-300 kD range while showing good resolutions in 400-600 kD, suggesting that a 5.5% gel might be needed. The range of fractionation seen is consistent with that in the single mode gels (see FIG. 7 (a)). However, over much of the size range, the resolution of the native free-flow gels is lower than that of the SDS free flow gels (compare FIG. 8 with FIG. 5). Due to the difficulty of correctly judging when to separate the two stages for elution, there is also an apparent preferential loss of proteins between 140 and ˜400 kD. For these reasons, native counter free-flow electrophoresis appears less effective than SDS counter free-flow, except for single mode applications targeting specific polypeptides.

Further optimization of the concentration and length of the upper gel column should achieve desired separation across the entire mass range. Also, for studying membrane protein complexes, the lower gel must be further optimized to increase resolution of smaller proteins as the membrane proteins and complexes tend to be much smaller.

We have noticed an extra tail trailing from fraction #32 and up in FIG. 9. Since the input sample did not contain protein or protein complexes bigger than the ones in fraction #28, we believe some aggregation must have occurred during the native gel electrophoresis. This is supported by the SDS gel in FIG. 9 (c), where the tailing lanes contained only the three most abundant proteins across all lanes. Aggregation is a well known possibility in native gel electrophoresis, especially when stacking gel is involved. Several possible ways for reducing or eliminating such aggregation were investigated, including the use of mild detergent in gel, reducing amount of protein loaded and native gel electrophoresis without stacking. It was found that the mild detergent had very little impact; the resolution without stacking was too poor; and the sample over-loading was the main cause of the aggregation. In fact, to reduce the aggregation, the sample load must be reduced by at least 10-fold, which makes it unsuitable for processing HIC fractions obtained in our current “tagless” scheme described in Example 6.

### Example 6

### High Throughput “Tagless” Strategy for Protein Separation and Purification

Proteins called based on co-eluting—the “guilty by association” principle. A complex must survive a comprehensive and complete separation and its components detected by mass spec. Complexes must be validated and confirmed by other assays.

This method can be high throughput, generic and sensitive—simultaneous identification and purification of many complexes, sensitive protein detection and large amount of material available.

A major rate limiting step in current mass spectrometry is sample preparation. A variety of methods have been used. In one approach, the purified complex is denatured and the constituent polypeptides separated prior to tryptic digestion (e.g. Gavin et al, 2002). Such methods, though, are inherently slow and difficult to automate. The present strategy is to employ a liquid chromatography “shot gun” approach, in which all the polypeptides in a fraction are digested with a protease and then reverse phase or two dimensional HLPC is used to separate peptides prior to analysis by MS/MS mass spectrometry (e.g. Butland et al, 2005).

The tagless strategy would basically comprise the following steps: A crude protein extract is fractionated successively by different chromatographic methods, such as by size exclusion chromatography, ion exchange chromatography, hydrophobic interaction, chromatofocusing. Selected fractions representing the full repertoire of proteins from each column are fractionated on the next column, and the process is repeated. Usually in these experiments after each column separation is performed, only those fractions that contain the protein being assayed are pooled and used for subsequent rounds of purification. If, however, fractions were to be separately taken that collectively represented the full repertoire of proteins present on a column and each were fractionated in parallel by a second chromatography method, and this process were to be repeated successively, a large number of fractions would be produced that would contain purified or partly purified and separated proteins and protein complexes. It is estimated that with an optimized strategy, it should be possible to detect by mass spectrometry the majority of water soluble stable complexes present at least 10 molecules per cell by analysis of around 10,000-20,000 chromatographic fractions.

The multi-channel gel electrophoresis system is planned to fill a large role in separation and fractionation of proteins and protein complexes.

The tagless strategy involves the analysis of sets of neighboring fractions. It would be prohibitively slow with current protocols to exhaustively analyze all detectable peptides in each fraction by MS/MS sequencing. To overcome this problem, a MALDI TOF/TOF mass spectrometer is used as the principle screening tool and link that to intelligent rapid data analyses algorithms that use information from each fraction and its neighbors to greatly reduce the number of peptides sequenced. By identifying ions in 1D MS spectra that derive from polypeptides whose identities have been determined in an earlier fraction, many ions can be eliminated from further MS/MS analysis. A critical advantage of MALDI over ESI that suits it for the present purposes is that it provides a ready means to archive samples, allowing quick and repeated return to the same fraction.

The high throughput method once implemented would allow screening a large number of samples that contain anywhere from a few to 20-30 polypeptides. However, it is unlikely that this pipeline will be 100% efficient in identifying all components of heteromeric complexes, and it will not provide quantitation on the relative stoichiometry of their polypeptide constituents. Therefore, once fractions containing sets of co migrating polypeptides have been defined, a set of more standard low throughput mass spectrometry methods will be used to provide a more complete characterization of the putative protein complexes. Once established, our combined high throughput screen and final polishing pipeline will be generally useful for many applications in high throughput mass spectrometry.

### Example 7

### Protein Separation

All separations were performed at 4° C. and protein elution was monitored by UV at 280 nm. E. coli lysates were prepared as previously described.¹⁹ Protein extracts at 20-50 mg/ml were separated by gel filtration on either a 1.6 cm×60 cm (120 ml) or a 2.6 cm×60 cm (320 ml) Sephacryl S-400 column equilibrated with buffer A (25 mM HEPES, 10% glycerol, 0.01% NP-40, 2 mM DTT) containing 100 mM NaCl; either 50 or 500 mg protein was loaded for the small- and large-scale experiments, respectively. The high-molecular-weight fraction from each column, represented by the first of the two major UV peaks eluting from the sizing column ( 1/7- 1/10 of the total protein eluted), was further separated by anion exchange chromatography using either an 8 ml or a 20 ml Mono Q column. The columns were developed with a NaCl gradient (from 100 mM to 600 mM) in buffer A that spanned 25 column volumes. For 8 ml and 20 ml columns, the flow rate was 2 ml/min and 4 ml/min with the collection of 25% and 10% column volume fractions, respectively.

A portion of the Mono Q fractions was subjected to a further gel filtration purification step using either a 1.0 cm×30 cm (24 ml) Superose 6 or a 0.46 cm×10 cm (1.7 ml) Source 15PHE 4.6/100 PE column. The Source 15PHE column was first equilibrated with buffer B (25 mM HEPES, 10% glycerol, 2 mM DTT) with 1 M (NH₄)₂SO₄. After sample loading, the column was developed with a linear gradient from Buffer B with 1 M (NH₄)₂SO₄ to Buffer B without (NH₄)₂SO₄.

Chromatography fractions were analyzed by SDS PAGE using the Criterion Precast gel system (Bio-Rad) 4-15% SDS PAGE gradient gels and 4-20% Native PAGE gradient gels were used. Gels were stained using a SilverQuest™ silver staining kit (Invitrogen).

### Example 8

### Protein Digestion and Labeling with iTRAQ Reagents

Selected portions of the anion exchange chromatography eluates were sampled for mass spectrometry analyses at a frequency of 25% or 50% column volumes. Specifically, one in two or one in six fractions were assayed, a total of seven and fifteen fractions for the small- and large-scale experiments, respectively. The protein content of the fractions was estimated by using the Bradford assay.²⁰ This information was used to ensure that protein digestion and derivatization for each experiment were performed at similar protein concentrations. Equal fraction volumes were digested and labeled when their respective protein concentrations were within 100% of each other. Otherwise, fraction volumes with equal protein concentrations were used as the starting material. Briefly, the proteins in each fraction were precipitated with acetone (6× volume excess), solubilized in 100 mM triethylammonium bicarbonate buffer (TEAB, pH 8.5) containing 0.1% SDS, reduced with tris-(2-carboxyethyl)phosphine (TCEP), alkylated with methyl methanethiosulfonate (MMTS) and digested with porcine trypsin (Pierce) at 37° overnight. The resulting tryptic peptide mixtures were derivatized with iTRAQ reagents in the TEAB buffer/80% ethanol for 1 hour at room temperature. The manufacturer's protocol for iTRAQ reagent labeling was followed, however, an approximate 4-5× higher iTRAQ reagent:protein ratio was used at the protein scale of ˜20-25 μg. Post-labeling, four consecutive Mono Q fractions, each tagged with a different iTRAQ reagent, were combined to generate a multiplexed sample; consecutive multiplexed samples shared one common fraction. The sample volume was reduced to ˜10-20 μL on a SpeedVac prior to one-step cation exchange chromatography which was carried out using the resin-containing cartridge and buffers provided by the manufacturer.¹⁷ The elutes that contained the peptide mixtures were concentrated to a volume 10-20 μL and stored at −20° C. prior to MALDI LC MS/MS analysis.

### Example 9

### LC MALDI MS/MS

A Pepmap C18 trap column and a nano-column (100 μm i.d., 15 cm length, Dionex/LC Packings), were used for desalting and reversed phase (RP) peptide separation, respectively. A 30 minute linear gradient from 2% B to 40% B was run at 500 nl/min flow rate, utilizing solvents A: 2% AcCN/0.1% trifluoroacetic acid (TFA) and B: 85% ACN/5% isopropanol, 1.0% TFA using an Ultimate LC System (Dionex/LC Packings). Reversed phase-separated peptides were collected directly onto a stainless steel MALDI target utilizing Probot (Dionex/LC Packings) spotting robot. Column elute was combined, in a mixing tee, with MALDI matrix (α-cyano-4-hydroxycinnamic acid, 6 mg/ml in 80% ACN/0.1% TFA/10 mM dibasic ammonium phosphate), containing 25 fmol/μl Glu-fibrinopeptide (GluFib) for internal calibration, delivered at 1 μl/min. Peptides were analyzed on a 4700 and 4800 Proteomics Analyzer mass spectrometer (Applied Biosystems/MDS Sciex) in the positive ion mode. The 4700 and 4800 Proteomics Analyzers were equipped with TOF/TOF™ ion optics and a 200 Hz NdYag laser.²¹ For collision-induced dissociation (CID), the collision cell was floated at 1 kV (4700) or 2 kV (4800), the resolution of the precursor ion selection was set to 200 and 300 FWHM for the 4700 and 4800 analyzers, respectively and air was used as the collision gas at 5×10⁻⁷ Torr. Automated acquisition of MS and MS/MS data was controlled by 4000 Series Explorer Software. Internal one-point calibration utilized m/z of monoisotopic molecular ion of GluFib that met the following acceptance criteria: S/N 50, mass error 50 ppm; when the acceptance criteria were not met, default calibration based on a plate model algorithm (Applied Biosystems) was employed.

Typical mass accuracy was within 10 ppm and 50 ppm for the internal and default calibration, respectively. Automated MS/MS data analysis was performed utilizing GPS Explorer software 3.5 with MASCOT 2.1.0 (Matrix Science) software for protein identification and quantitation of iTRAQ reporter ions. The following criteria were employed for generation of MS/MS peak list: S/N 5, m/z 50 to −20 from a precursor molecular ion, 50 peaks per 200 Da, a maximum number of peaks 80. E. coli taxonomy within Swiss Prot protein database, release 48.0 of 13 Sep. 2005 and release 49.6 of 2 May 2006, was interrogated for the data sets generated on 4700 for all 15 fractions and on 4800 for the first 10 fractions, respectively.

The following search parameters were utilized: precursor mass tolerance 50 ppm; fragment mass tolerance 0.15 Da; tryptic digestion with 2 missed cleavages; fixed modifications: S-MMTS, K-iTRAQ and N-term iTRAQ; variable modifications: deamidation (Asn and Gln); Met-sulfoxide. GPS Confidence Interval (C.I. %) of 95% was used as the acceptance criteria and hence identification of each polypeptide was based upon at least one peptide that scored above a threshold value set by the Mascot search engine to indicate identity or extensive homology of proposed sequence at p<0.05. The reported protein list was manually updated to reflect the UniProt protein entry names and accession numbers (release 53.2 of 26 Jun. 2007); EcoCyc database²² (http://ecocyc.org/) was utilized to facilitate this process. Average relative ratios were calculated for each polypeptide using the GPS Explorer 3.5 algorithm without invoking a “bias” correction option. Only peptides that were completely labeled with iTRAQ at N-termini and lysines and whose individual relative ratios were different from zero were considered while calculating protein average. The outliers were automatically excluded.

### Example 10

### Evaluation of Quality of Quantitation Data and Biological Reliability of Measurements

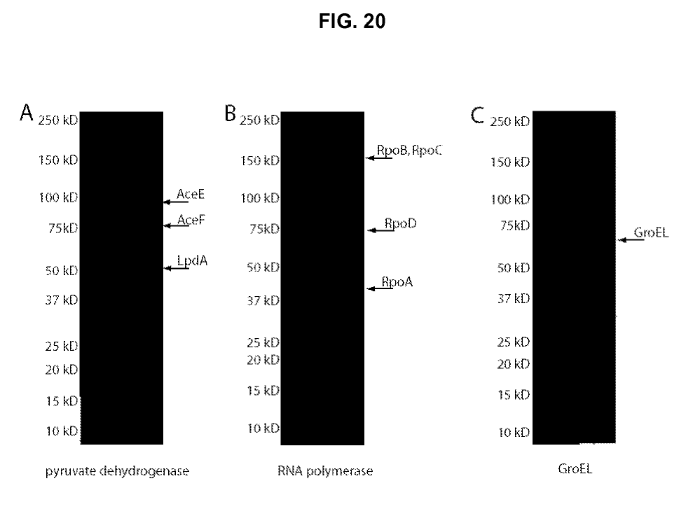

To evaluate the extent of side reactions, the data were re-analyzed by interrogation of the same database using the same parameters as described above with the exception of iTRAQ settings, this time specifying a flexible rather than a fixed modification type and allowing for tyrosine derivatization. Only a limited number of under-derivatized peptides was revealed and no hits carrying iTRAQ-labeled tyrosine were found. In order to minimize the number of overlapping precursors, precursor ion selection for MS/MS data acquisition was performed at the resolution as high as possible without significantly jeopardizing sensitivity and a filter of a minimum of 200 resolution between a target precursor and potential non-related molecular ions was applied. Nevertheless, given the complexity of the sample and the limitation of the TOF/TOF precursor ion selection window it is inevitable that some of the quantitation data might have been adversely affected by interfering ions. A potential presence of multiple precursors was not addressed by the GPS software and no systematic examination of all the data was undertaken to evaluate the extent of the possible problem. However, a limited number of MS and MS/MS spectra, predominantly those derived from proteins represented by a small number of peptides, were examined manually and in the great majority of cases, no significant level of unexplained (product ion) signals was observed (FIG. 20). The inventors have also examined a number of outliers among multiple peptides representing abundant proteins and only a small number of them could be possibly explained by the presence of a detectable interfering ion close to the intended precursor (data not shown). Reproducibility of the methods described in this study is presented and discussed infra. The aim of the study was to compare the results of the tagless strategy with the known information on protein complexes and protein-protein interactions in the model organism, E. coli. It was not intended to be a discovery study and hence no attempt was made to validate any unknown, putative, complexes that might have been detected using a clustering algorithm (Table 5).

Not shown is evidence of identification of category [ID1] polypeptides that were matched on the basis of a single peptide: annotated MS/MS spectra. Each MS/MS spectrum was accompanied by the following information: polypeptide ID # (see Table 3), polypeptide code name and entry name, peptide sequence, experimental m/z of molecular ion and an error of mass measurement (in ppm). Theoretically expected masses of product ions are shown in the tables (in insests) and fragments that were detected are highlighted. The spectra were processed by Data Explorer 1.9 tools: baseline correction and noise filtering. The mass errors of the reported peptides were consistent with errors of other confidently identified peptides detected at the same MALDI target spots.

### Example 11

### Deriving Polypeptide Elution Profiles

The final average relative ratios for the individual polypeptide components of each multiplexed set were normalized to the same fraction volume. Separate multiplexes were aligned and elution profiles for each polypeptide (over the entire chromatographic run) were drawn using the following procedure. The absolute values of the relative ratios measured for each polypeptide in a fraction that was shared between two adjacent multiplexed samples were equalized using the value of the precedent fraction as a reference point. The relative ratios of the same polypeptide in the remaining three fractions of the multiplexed sample were then adjusted to maintain the original ratio. Finally, relative polypeptide abundance was determined by arbitrarily assigning a value of 1.0 to the apex of each polypeptide elution peak and normalizing all other data points accordingly. When multiple apices of a contiguously eluting polypeptide were seen, the highest value within the original peak profile was used as a reference point and assigned a value of 1.0. By definition, all other apices had values that were less than 1.0 and their relative ratios corresponded to the abundance of the same polypeptide in fractions that were collected at different times. In this schema, local differences in apex values for different polypeptides were a consequence of the arbitrary method that was used to calculate elution profile and hence, they had no physical meaning. After normalizing and scaling, elution profiles were plotted as 2-D graphs where the ordinate values corresponded to the relative abundances of the component polypeptides in each fraction and the abscissa values represented the order in which the fractions eluted.

### Example 12

### Polypeptide Clustering

To identify putative protein complexes, a comparison of polypeptide elution profiles was performed within all the fractions where the polypeptide was observed. Average relative ratios calculated for each polypeptide by the GPS Explorer 3.5 algorithm that were normalized and scaled, as described above, were employed for clustering analysis. The first step was to identify all valid profile peaks using the following process: (i) find the center, left and right edges for all elution peaks for all polypeptides using a simple peak detection algorithm developed in our laboratory (ii) filter out the noise. The latter was accomplished by examination of the peak intensity ratios relative to the highest peak in the same polypeptide profile (R1) and relative to the intensities of its own left and right edges (R2). If any of the ratios, R1 and/or R2, were below the threshold (R1≦0.15 and R2≦1.20), the peak was classified as noise. The R1 and R2 threshold values are dependent on the data complexity and quality and might need further tuning in the future as the data size grows. Once a set of elution peaks of all polypeptides was established, Pearson correlation coefficients between any two peaks that overlap significantly were calculated. In this work, Pearson correlation coefficients were used as a measure of similarity between two peaks, see the formula below where (x₁, x₂, x₃, . . . xₙ) and (y₁, y₂, y₃, . . . yₙ) are normalized intensities of peaks x and y across fractions 1, 2, . . . n, and X and Y are their average intensities across all n fractions, respectively.

$$r = \frac{{\sum }_{i = 1}^{n} ( {x}_{i} - X ) ( {y}_{i} - Y )}{\sqrt{{\sum }_{i = 1}^{n} {( {x}_{i} - X )}^{2} {\sum }_{i = 1}^{n} {( {y}_{i} - Y )}^{2}}}$$

The clustering analysis routine was based on an algorithm originally developed for evaluation of gene expression profiles (http://genetics.stanford.edu/˜sherlock/cluster.html). This algorithm was customized to accommodate our polypeptide elution profile data. Mathematical averages of coefficient values of clustered peaks were used as the metrics for similarity measurement. Based on these criteria, a putative complex is called if the average Pearson coefficient of a cluster of polypeptides exceeds a threshold value of 0.92.

### Example 13

### A Proof-of-Principle Demonstration

To provide a first proof-of-principle that the strategy allows protein complexes to be detected, the inventors first quantitated the relative levels of five RNA polymerase subunits across a series of Mono Q anion exchange fractions.¹⁶ In this and subsequent iTRAQ analyses, fractions were sampled at a frequency such that they were separated by at least one fraction and by no more that 25%-50% of a column volume as this was found to provide sufficient resolution to detect co-migration of polypeptides belonging to known complexes. The fractions themselves were quite heterogeneous, being derived from only two chromatography steps and contained a broad mixture of many polypeptides (FIG. 4A). None the less, despite this crude fractionation, between six to thirty tryptic peptides were detected for the five known subunits of RNA polymerase. The iTRAQ quantification showed that the individual peptides derived from a given polypeptide gave similar, albeit, not overlapping relative concentration profiles across the fractions (FIG. 4B). The inventors also observed similar variation between peptides in model studies on standard proteins (unpublished data), suggesting that this variation resulted from differential rates of peptide generation during tryptic digestion and/or losses during sample processing, rather than being indicative of structural heterogeneity within the proteins present in each fraction. While relative abundances of some of tryptic peptides varied significantly from the mean, elution profiles based upon their relative ratios differed from the average elution profile in amplitude but not in localization of apices (FIG. 4B). Hence, if only few peptides are detected due to low polypeptide abundance, it is desirable to monitor the same set of tryptic peptides representing the polypeptide of interest in all fractions that are analyzed. When a sufficient number of peptides are detected, the mean data for multiple peptides should be the best guide to the relative abundance of each polypeptide. Indeed the averaged profiles for all five polymerase components were very similar to each other (FIG. 4C), consistent with the known tight association of these five polypeptides.

The above results suggest that iTRAQ quantitation is sufficiently accurate to detect co-migrating complex components. Since the fractions analyzed contain far more proteins than the highly purified fractions envisioned being assayed in our finalized tagless strategy protocol, the fact that the iTRAQ-based method was effective in these less than optimal circumstances was encouraging.

### REFERENCES

(1) Buchanan, M. V.; Larimer, F. W.; Wiley, H. S.; Kennel, S. J.; Squier, T. J.; Ramsey, J. M.; Rodland, K. D.; Hurst, G. B.; Smith, R. D.; Xu, Y.; Dixon, D.; Doktycz, M. J.; Colson, S.; Gesteland, R.; Giometti, C.; Young, M.; Giddings, M. Genomes to Life “Center for Molecular and Cellular Systems”: a research program for identification and characterization of protein complexes. Omics 2002, 6, 287-303.
(2) McHenry, C. S.; Crow, W. DNA polymerase III of Escherichia coli. Purification and identification of subunits. J Biol Chem 1979, 254, 1748-1753.
(3) Srere, P. A.; Mathews, C. K. In Guide to Protein Purification; Deutscher, M. P., Ed.; Academic Press: San Diego, 1990; Vol. 182, pp 539-551.
(4) Austin, R. J.; Biggin, M. D. Purification of the Drosophila RNA polymerase II general transcription factors. Proc Natl Acad Sci USA 1996, 93, 5788-5792.
(5) Link, A. J.; Fleischer, T. C.; Weaver, C. M.; Gerbasi, V. R.; Jennings, J. L. Purifying protein complexes for mass spectrometry: applications to protein translation. Methods 2005, 35, 274-290.
(6) Balbo, A.; Minor, K. H.; Velikovsky, C. A.; Mariuzza, R. A.; Peterson, C. B.; Schuck, P. Studying multiprotein complexes by multisignal sedimentation velocity analytical ultracentrifugation. Proc Natl Acad Sci USA 2005, 102, 81-86.
(7) Camacho-Carvajal, M. M.; Wollscheid, B.; Aebersold, R.; Steimle, V.; Schamel, W. W. Two-dimensional Blue native/SDS gel electrophoresis of multi-protein complexes from whole cellular lysates: a proteomics approach. Mol Cell Proteomics 2004, 3, 176-182.
(8) Rout, M. P.; Aitchison, J. D.; Suprapto, A.; Hjertaas, K.; Zhao, Y.; Chait, B. T. The yeast nuclear pore complex: composition, architecture, and transport mechanism. J Cell Biol 2000, 148, 635-651.
(9) Rigaut, G.; Shevchenko, A.; Rutz, B.; Wilm, M.; Mann, M.; Seraphin, B. A generic protein purification method for protein complex characterization and proteome exploration. Nat Biotechnol 1999, 17, 1030-1032.
(10) Puig, O.; Caspary, F.; Rigaut, G.; Rutz, B.; Bouveret, E.; Bragado-Nilsson, E.; Wilm, M.; Seraphin, B. The tandem affinity purification (TAP) method: a general procedure of protein complex purification. Methods 2001, 24, 218-229.
(11) Winkler, G. S.; Lacomis, L.; Philip, J.; Erdjument-Bromage, H.; Svejstrup, J. Q.; Tempst, P. Isolation and mass spectrometry of transcription factor complexes. Methods 2002, 26, 260-269.
(12) Burckstummer, T.; Bennett, K. L.; Preradovic, A.; Schutze, G.; Hantschel, O.; Superti-Furga, G.; Bauch, A. An efficient tandem affinity purification procedure for interaction proteomics in mammalian cells. Nat Methods 2006, 3, 1013-1019.
(13) Gavin, A. C.; Aloy, P.; Grandi, P.; Krause, R.; Boesche, M.; Marzioch, M.; Rau, C.; Jensen, L. J.; Bastuck, S.; Dumpelfeld, B.; Edelmann, A.; Heurtier, M. A.; Hoffman, V.; Hoefert, C.; Klein, K.; Hudak, M.; Michon, A. M.; Schelder, M.; Schirle, M.; Remor, M.; Rudi, T.; Hooper, S.; Bauer, A.; Bouwmeester, T.; Casari, G.; Drewes, G.; Neubauer, G.; Rick, J. M.; Kuster, B.; Bork, P.; Russell, R. B.; Superti-Furga, G. Proteome survey reveals modularity of the yeast cell machinery. Nature 2006, 440, 631-636.
(14) Krogan, N. J.; Cagney, G.; Yu, H.; Zhong, G.; Guo, X.; Ignatchenko, A.; Li, J.; Pu, S.; Datta, N.; Tikuisis, A. P.; Punna, T.; Peregrin-Alvarez, J. M.; Shales, M.; Zhang, X.; Davey, M.; Robinson, M. D.; Paccanaro, A.; Bray, J. E.; Sheung, A.; Beattie, B.; Richards, D. P.; Canadien, V.; Lalev, A.; Mena, F.; Wong, P.; Starostine, A.; Canete, M. M.; Vlasblom, J.; Wu, S.; Orsi, C.; Collins, S. R.; Chandran, S.; Haw, R.; Rilstone, J. J.; Gandi, K.; Thompson, N. J.; Musso, G.; St Onge, P.; Ghanny, S.; Lam, M. H.; Butland, G.; Altaf-Ul, A. M.; Kanaya, S.; Shilatifard, A.; O'Shea, E.; Weissman, J. S.; Ingles, C. J.; Hughes, T. R.; Parkinson, J.; Gerstein, M.; Wodak, S. J.; Emili, A.; Greenblatt, J. F. Global landscape of protein complexes in the yeast Saccharomyces cerevisiae. Nature 2006, 440, 637-643.
(15) Butland, G.; Peregrin-Alvarez, J. M.; Li, J.; Yang, W.; Yang, X.; Canadien, V.; Starostine, A.; Richards, D.; Beattie, B.; Krogan, N.; Davey, M.; Parkinson, J.; Greenblatt, J.; Emili, A. Interaction network containing conserved and essential protein complexes in Escherichia coli. Nature 2005, 433, 531-537.
(16) Dong, M.; Biggin, M. D.; Williams, K.; Dixon, S. E.; Yang, L. L.; Fisher, S. J.; Hall, C. S.; Jin, J.; Witkowska, H. E. Multi-dimensional Orthogonal Separation and iTRAQ™ Reagent Tracking: A Genome-wide “Tagless” Strategy for Isolation and Detection of Soluble Bacterial Protein Complexes. Presented at the 54 ASMS Conference on Mass Spectrometry and Allied Techniques, 28 May-1 Jun., 2006, Seattle, Wash.
(17) Ross, P. L.; Huang, Y. N.; Marchese, J. N.; Williamson, B.; Parker, K.; Hattan, S.; Khainovski, N.; Pillai, S.; Dey, S.; Daniels, S.; Purkayastha, S.; Juhasz, P.; Martin, S.; Bartlet-Jones, M.; He, F.; Jacobson, A.; Pappin, D. J. Multiplexed Protein Quantitation in Saccharomyces cerevisiae Using Amine-reactive Isobaric Tagging Reagents. Mol Cell Proteomics 2004, 3, 1154-1169.
(18) Andersen, J. S.; Wilkinson, C. J.; Mayor, T.; Mortensen, P.; Nigg, E. A.; Mann, M. Proteomic characterization of the human centrosome by protein correlation profiling. Nature 2003, 426, 570-574.
(19) Garczarek, F.; Dong, M.; Typke, D.; Witkowska, H. E.; Hazen, T. C.; Nogales, E.; Biggin, M. D.; Glaeser, R. M. Octomeric pyruvate-ferredoxin oxidoreductase from Desulfovibrio vulgaris. J Struct Biol 2007, 159, 9-18.
(20) Bradford, M. M. A rapid and sensitive method for the quantitation of microgram quantities of protein utilizing the principle of protein-dye binding. Anal Biochem 1976, 72, 248-254.
(21) Medzihradszky, K. F.; Campbell, J. M.; Baldwin, M. A.; Falick, A. M.; Juhasz, P.; Vestal, M. L.; Burlingame, A. L. The characteristics of peptide collision-induced dissociation using a high-performance MALDI-TOF/TOF tandem mass spectrometer. Anal Chem 2000, 72, 552-558.
(22) Keseler, I. M.; Collado-Vides, J.; Gama-Castro, S.; Ingraham, J.; Paley, S.; Paulsen, I. T.; Peralta-Gil, M.; Karp, P. D. EcoCyc: a comprehensive database resource for Escherichia coli. Nucleic Acids Res 2005, 33, D334-337.
(23) Zieske, L. R. A perspective on the use of iTRAQ reagent technology for protein complex and profiling studies. J Exp Bot 2006, 57, 1501-1508.
(24) Bondarenko, P. V.; Chelius, D.; Shaler, T. A. Identification and Relative Quantitation of Protein Mixtures by Enzymatic Digestion Followed by Capillary Reversed-Phase Liquid Chromatography-Tandem Mass Spectrometry. Anal. Chem. 2002, 74, 4741-4749.
(25) Wang, W.; Zhou, H.; Lin, H.; Roy, S.; Shaler, T. A.; Hill, L. R.; Norton, S.; Kumar, P.; Anderle, M.; Becker, C. H. Quantification of Proteins and Metabolites by Mass Spectrometry without Isotopic Labeling or Spiked Standards. Anal. Chem. 2003, 75, 4818-4826.
(26) Liu, H.; Sadygov, R. G.; Yates, J. R. A Model for Random Sampling and Estimation of Relative Protein Abundance in Shotgun Proteomics. Anal. Chem. 2004, 76, 4193-4201.
(27) Zhang, B.; VerBerkmoes, N.C.; Langston, M. A.; Uberbacher, E.; Hettich, R. L.; Samatova, N. F. Detecting differential and correlated protein expression in label-free shotgun proteomics. J Proteome Res 2006, 5, 2909-2918.
(28) Bublitz, R.; Kreusch, S.; Ditze, G.; Schulze, M.; Cumme, G. A.; Fischer, C.; Winter, A.; Hoppe, H.; Rhode, H. Robust protein quantitation in chromatographic fractions using MALDI-MS of tryptic peptides. Proteomics 2006, 6, 3909-3917.
(29) Bantscheff, M.; Schirle, M.; Sweetman, G.; Rick, J.; Kuster, B. Quantitative mass spectrometry in proteomics: a critical review. Anal Bioanal Chem 2007.
(30) Hartman, N. T.; Sicilia, F.; Lilley, K. S.; Dupree, P. Proteomic complex detection using sedimentation. Anal Chem 2007, 79, 2078-2083.
(31) Dunkley, T. P.; Watson, R.; Griffin, J. L.; Dupree, P.; Lilley, K. S. Localization of organelle proteins by isotope tagging (LOPIT). Mol Cell Proteomics 2004, 3, 1128-1134.
(32) Sadowski, P. G.; Dunkley, T. P.; Shadforth, I. P.; Dupree, P.; Bessant, C.; Griffin, J. L.; Lilley, K. S. Quantitative proteomic approach to study subcellular localization of membrane proteins. Nat Protoc 2006, 1, 1778-1789.
(33) Higgins, N. P.; Peebles, C. L.; Sugino, A.; Cozzarelli, N. R. Purification of subunits of Escherichia coli DNA gyrase and reconstitution of enzymatic activity. Proc Natl Acad Sci USA 1978, 75, 1773-1777.
(34) Eisen, M. B.; Spellman, P. T.; Brown, P. O.; Botstein, D. Cluster analysis and display of genome-wide expression patterns. Proc Natl Acad Sci USA 1998, 95, 14863-14868.
(35) Dong, M.; Liu, H.; Allen, S.; Hall, S.C. H.; Fisher, S. J.; C, H. T.; Geller, Jil T; Singer, M. E.; Yang, L. L.; Jin, J.; Biggin, M. D.; Witkowska, H. E. Methodological Refinements in iTRAQ™ Reagent-Based “Tagless” Strategy of Identification and Purification of Soluble Protein Complexes in Bacteria. Presented at the 8th International Symposium on Mass Spectrometry in the Health and Life Sciences: Molecular and Cellular Proteomics, Aug. 19-23, 2007, San Francisco, Calif.

### Example 13

### Survey of Large Protein Complexes in D. Vulgaris

As part of a larger program to characterize and image the ensemble of macromolecular complexes in Desulfovibrio vulgaris Hildenborough (PCAP at LBNL), a bacterium of potential use in bioremediation of soils contaminated by toxic heavy metals (8-10), we have undertaken a survey of the most abundant complexes that are large enough to be distinguishable from one another within tomographic reconstructions of single cells. A total of 15 different macromolecular complexes with particle weights of at least 400 kDa were isolated by a “tagless” strategy (11), which is “unbiased” in the sense that it makes no prior assumptions about which protein complexes should be purified. Instead, purification used a high-throughput pipeline that includes differential solubility in ammonium sulfate, ion-exchange chromatography, hydrophobic interaction chromatography, and size-exclusion chromatography. In addition, DvH ribosomes were isolated by a special-purpose protocol similar to that used for the purification of 70 S E. coli ribosomes.

Because the collection of multiprotein complexes within DvH had not been cataloged, we used a “tagless” method to purify, identify, and structurally characterize those complexes that remain stable upon cell lysis. This method makes no assumptions about what proteins might exist in the form of multiprotein complexes, or what the subunit stoichiometries and quaternary structures of these complexes should be. Instead, comigrating protein subunits are separated on the basis of their physical properties, and the constituent polypeptides are identified by mass spectroscopy. The resulting proteomic survey reported here is intentionally limited to complexes with molecular mass greater than; ≈400 kDa and copy number greater than ≈100 per cell, because these would be the easiest ones to identify in EM tomograms due to their size and abundance. One of the complexes (phosphoenolpyruvate synthase) that was isolated in this way proved, however, to be a 265 kDa homodimer, which eluted during size-exclusion chromatography (SEC) as an ≈370 kDa particle. Electron microscopy of this particle subsequently showed it to have an elongated shape, thus explaining its anomalously high apparent molecular weight in SEC.

The biochemical identities and subunit compositions of the 15 “largest, most abundant particles” that we found within DvH are given in Table 6. Three of this set proved to be homo-oligomeric complexes of proteins (DVU0631, DVU0671, and DVU1012, respectively) for which no biochemical function could be identified or for which only weak similarity to proteins with known functions could be detected. Ten of the remaining 12 protein complexes whose biochemical functions could be identified with confidence are ones involved either in energy metabolism or in pathways of intermediary metabolism. The two remaining particles, GroEL and RNA polymerase, were already expected to be among the set of abundant particles in the desired size range.

Three-dimensional reconstructions were obtained at a resolution of 3 nm or better for 70 S ribosomes in addition to 7 of 15 complexes purified by the high-throughput, tagless pipeline. In addition, the values of particle weight obtained by size-exclusion chromatography and native gel electrophoresis were used to estimate the subunit stoichiometries of those complexes for which single-particle EM reconstructions were not successful. Images of the eight 3D reconstructions that were successful, images not shown, illustrate the fact that each such particle has a characteristic size and shape by which it could be identified. The extent to which diverse particles can be distinguished on the basis of their sizes and shapes supports the proposal that it will be possible to identify and localize a large number of different macromolecular complexes within cryo-EM tomograms, provided that these are obtained with a resolution in the range of 3 nm or better.

The preparation of samples for electron microscopy does not always produce specimens suitable for obtaining three-dimensional reconstructions, and as a result structures were not obtained for 8 of 15 complexes purified by the tagless approach. In some cases it appeared that the particles might be inherently flexible or polymorphic in structure, but in other cases we believe that the particles were easily damaged at some step during preparation for electron microscopy. Our success rate in producing informative 3D reconstructions is nevertheless at least 10 times higher than that reported in an earlier survey of complexes in the yeast proteome (12), possibly because our focus on characterizing only the largest such complexes. In addition, we took further time to optimize the details of preparing EM grids for each type of protein whenever the initial results looked promising, but there nevertheless was more heterogeneity than expected. Although the fraction of purified complexes for which we were able to get good three-dimensional reconstructions was thus relatively high, we believe that generic improvements in preparing single-particle samples for electron microscopy (rather than further biochemical purification of samples) could further improve the success rate and throughput.

Apart from GroEL and the 70 S ribosome, all of the remaining complexes whose biochemical identities can be assigned with confidence were found to have subunit stoichiometries or quaternary structures that are not fully conserved, even within bacteria, as is shown in column 7 and 8 of Table 6. The extent to which quaternary structures vary between different bacteria is quite surprising, because tertiary structure is normally well conserved over great evolutionary distance and because the quaternary structures of some homomeric (e.g. GroEL) and heteromeric (e.g. RNAP core enzyme) protein complexes have been found to be conserved over long evolutionary distances.

The striking nature of our observation is highlighted by a further description of the following four examples. First, the majority of DvH RNAP II is purified as an unusual complex containing two copies of both the core enzyme and NusA (particle E shown in FIG. 1), a particle that has not been seen previously in other bacteria. Second, DvH pyruvate:ferredoxin oxidoreductase (PFOR) is an octomer (particle B in FIG. 1), but in another species of the same genus, Desulfovibrio africanus, it is a dimer (14). As we have reported previously (15), the insertion of a single valine residue into a surface loop of the dimer appears to account for the assembly of the DvH protein into the higher oligomer. Third, while lumazine synthase (also known as riboflavin Synthase® subunit) forms an icosahedral complex in DvH as it does in B. subtilis (16) and Aquifex aeolicus (17), the pentameric subunit is rotated by about 30 degrees relative to its orientation in the previously reported icosahedral structures, as is shown in FIG. 2. As a result of this rotation, the diameter of the DvH icosahedron is increased, and the interaction interface between pentamers is clearly not conserved. Instead, the vertices of the DvH pentamers make head-to-head contact with one another at the icosahedral 3-fold axis rather than the side-by-side contact between edges of the pentamers that is seen in the previously described structures. Fourth, a DvH homolog of the carbohydrate phosphorylase family is a ring-shaped complex, as is shown in FIG. 3. Although it was not possible to obtain a 3-D reconstruction for this particle at a resolution high enough to determine the subunit stoichiometry, its particle weight on size-exclusion chromatography suggests that it is at least a hexamer (Table 1). Since previously described members of the carbohydrate phosphorylase family are either monomers or dimers, these ring-shaped particles represent a novel quaternary structure for this family.

Cell culture and biomass production. Protein complexes were isolated from cells grown as mid-logarithmic cultures in 5-L or 400-L fermentors, which were run as turbidostats. As mentioned above, up to 4 orthogonal separation methods were used to purify multiprotein complexes solely on the basis of differences in their physical properties. The subunit compositions of samples containing purified complexes that ran on native-gel electrophoresis as predominantly a single band with Mr>400 k were characterized by SDS PAGE, and mass spectroscopy was used to identify the component proteins. Further details about cell growth, the purification of each respective complex, and the identification of proteins by mass spectroscopy are provided in Han et al., “Survey of large protein complexes in D. vulgaris reveals great structural diversity,” Proc Natl Acad Sci USA. 2009 Sep. 29; 106(39): 16580-16585, published online 2009 Sep. 11 hereby incorporated by reference in its entirety for all purposes.

D. vulgaris Hildenborough (DvH) (ATCC 29579) was obtained from the American Type Culture Collection (Manassas, Va.). A defined lactate-sulfate medium, LS4D (3) is used in all cultures. The medium is sterilized by autoclaving for 45 minutes at 121° C. Before inoculation, phosphate, vitamins and reducing agent (titanium citrate) are added to the medium. Stock cultures of DvH were prepared by growing the ATCC culture to log phase, and storing at −80° C. Starter culture is prepared inside an anaerobic chamber (Coy Laboratory Products, Inc., Grass Lake, Mich.) using stock culture at a ratio of 1 ml stock/100 ml LS4D. The starter culture is incubated at 30° C. and allowed to grow for 48 hrs to log phase (optical density at 600 nm of ˜0.3-0.4; ˜3×10₈ cells/ml). From the starter culture, a 10% subculture for inoculating the production culture is made in LS4D, in the anaerobic chamber, and incubated at 30° C. until log phase growth is reached (around 15 hours).

The production culture is grown in 5 L customized fermentors (Electrolab, Fermac 360, United Kingdom), run as turbidostats. PEEK headplates and agitators were specially manufactured so that there are no metallic wetted parts. The fermentor is autoclaved with 4.5 L LS4D medium and cooled on the bench under a nitrogen gas blanket. Once cooled, vitamins, phosphate and reducing agent are injected to the fermentor, followed by ten percent subculture (500 mL). The fermentor is continuously agitated at 200 rpm, maintained at 30° C., with nitrogen flowing through the headspace at 100 mL/min. Once log phase is reached, fresh medium is pumped to the fermentor at a dilution rate of 0.3 l/hr, maintaining an optical density of 0.6 (at 600 nm). The effluent passes through a chilling coil and is collected in a 20 L carboy where the temperature in maintained at 2-4° C. Effluent is collected over 12-15 hours, and then centrifuged at 11,000 g for 10 minutes, with refrigeration at 4° C. (Beckman Coulter, Avanti J-25). The supernatant is discarded, and the pellets are stored at −80° C. until further processing.

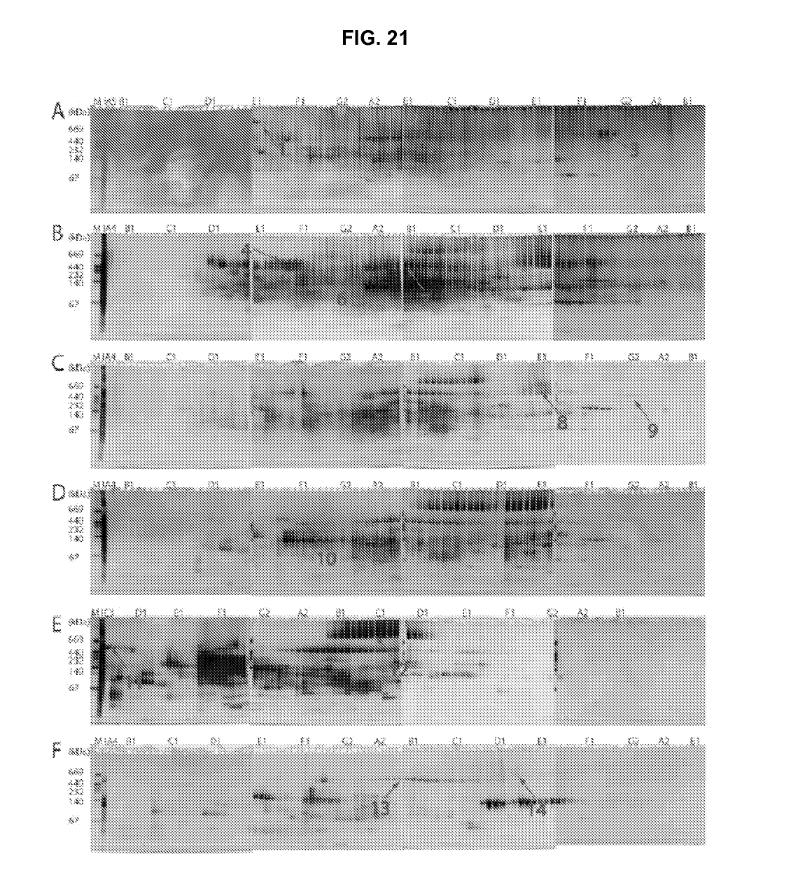

Purification of protein complexes Overview. The tagless purification strategy was based on previously described in the Examples above. All complexes were purified from cells derived from either a small scale culture of 20 L or a large scale culture of 400 L. Proteins were first bound to and then batch eluted from a QSepharose clean up column to remove many nonprotein impurities. 400 L scale preparations were then fractionated into six parts by ammonium sulfate precipitation. The ammonium sulfate fractions from the large preparation or the cleaned up small scale preparations were then fractionated by MonoQ chromatography. All the fractions from each MonoQ column were analyzed by both native and SDS PAGE to identify abundant protein bands that migrated at approximately 400 kDa or greater (FIG. 21). In addition, proteins that did not bind to the Q-Sepharose cleanup column were further fractionated by size exclusion chromatography (SEC) and then analyzed by SDS PAGE (FIG. 22). Fractions containing each putative protein complex were pooled and subjected to hydrophobic interaction chromatography (HIC) and/or SEC until sufficiently pure for EM analysis. 15 protein complexes were successfully purified to at least 75% purity as estimated by SDS PAGE (FIGS. 21 and 22); a further 5 complexes proved either to migrate at less than 300 kDa on an SEC column or to be duplicates of other protein bands and thus were not analyzed by EM. Suitable fractions were buffer exchanged into 10 mM HEPES, pH 7.6, 2 mM DTT, 0.01% NP-40 for EM as described previously.

Experimental Methods. Extracts were prepared as described previously in Garczarek F, et al. (2007) Octomeric pyruvate-ferredoxin oxidoreductase from Desulfovibrio vulgaris, Journal of Structural Biology 159(1):9-18 and hereby incorporated by reference. 20 L bacterial cultures yielded crude extracts of 340 mg of protein and 400 L cultures yielded 10 g of protein. Chromatography was done using a AKTA FPLC system. All chromatography columns and media were from GE Healthcare. All separations were performed at 4° C. except hydrophobic interaction chromatography (HIC), which was run at room temperature. The concentrations of proteins were monitored by UV light at 280 nm. Mixtures of two buffers were used for ion exchange chromatography (IEC) and HIC. For IEC, buffer A contained 25 mM HEPES pH 7.6, 0 M NaCl, 10% (v/v) glycerol, 2 mM DTT, 0.01% (v/v) NP-40 and buffer B contained buffer A plus 1 M NaCl. For HIC, buffer A′ contained 25 mM HEPES pH 7.6, 10% (v/v) glycerol, 2 mM DTT and buffer B′ contained buffer A′ plus 2 M (NH₄)₂SO₄. For SEC, the buffer used contained 25 mM HEPES pH 7.6, 0.05 M NaCl, 10% (v/v) glycerol, 2 mM DTT, 0.01% (v/v) NP-40.

Q-Sepharose clean-up: Protein extract supernatants were loaded onto either a 1.6×20 cm (small scale) or 5.0×30 cm (large scale) Q-Sepharose Fast Flow column equilibrated with 5% buffer B, and the bound proteins were eluted together with 50% buffer B. All fractions containing significant amounts of protein were pooled. The total protein amount obtained was 240 mg and 7 g for the small and large scale preparations respectively.

Ammonium sulfate precipitation: After the Q-Sepharose clean-up step, the large scale extract was fractionated into 6 parts by ammonium sulfate precipitation: 0-38%, 38-48%, 48-53%, 53-57%, 57-63% and greater than 63% ammonium sulfate saturation. Each cut, which contained between 568 mg to 1028 mg protein, was desalted into 5% 5 buffer B by buffer exchange using a G25 desalting column (5.0×30 cm).

Anion exchange chromatography: The post clean-up step small scale extracts were applied to a 20 ml 1.6×10 cm, 20 ml MonoQ column. Each desalted ammonium sulfate precipitation cut from large scale preparations was loaded to a 3.5×10 cm, 96 ml MonoQ column. All MonoQ columns were pre-equilibrated with 5% buffer B and developed with a linear gradient from 5% to 50% buffer B in 25 column volumes. For the 20 ml and 96 ml columns, the flow rates were 4 ml/min and 10 ml/min and fraction sizes were 4 ml and 24 ml respectively.

Protein complex survey: To quickly locate high abundance large molecular weight protein complexes, the Mono Q fractions were analyzed by native PAGE (e.g. FIG. 21). In addition, those proteins that did not bind the Q-Sepharose column were fractionated by SEC and the resulting fractions also analyzed by native PAGE (images not shown). 20 strong protein bands, which migrated at approximately 400 kDa or greater on native PAGE were picked and subjected for further purification. The fractions containing these chosen target complexes were further fractionated by HIC and/or SEC until EM grade purity were reached. Specific details of the HIC and SEC steps for each factor are described in Han et al, Proc Natl Acad Sci USA. 2009 Sep. 29; 106(39): 16580-16585.

Protein complex molecular weight calculation: The molecular weights of purified protein complexes were determined from their migration on a 1.0×30 cm Superose6 column or a 1.6×60 Superdex200 column in SEC buffer. The molecular weight standards used to calibrate the SEC column were BSA (67 kDa), aldolase (158 kDa), catalase (223 kDa), ferritin (440 kDa), and thyroglobulin (669 kDa).

Protein copy number estimation: The copy numbers of protein complexes per cell listed in Table 1 were estimated from the amount of protein in the flow through of the QSepharose cleanup column and the Mono Q fractions; the estimated yield of total protein present after chromatography; and the number of cells used in the preparation. The amount of each complex in the MonoQ fractions or the Q-Sepharose flow through was estimated from native PAGE by comparing the target protein bands with known amounts of a BSA standard.

Electrophoresis and silver staining: Chromatographic fractions were analyzed by PAGE using Criterion Precast gels (Bio-Rad): 4-15% gradient gels for native PAGE and 4-20% gradient gels for SDS PAGE. Gels were stained using a SilverQuest™ silver staining kit (Invitrogen)

Identification of protein components by mass spectroscopy. Reagents used ACS/HPLC grade acetonitrile (AcCN) and HPLC water were from Honeywell Burdick & Jackson; trifluoroacetic acid (TFA) was from Pierce, Suprapur formic acid was from EMD Biosciences; sequencing grade modified porcine trypsin was from Promega; C18 ziptips and MultiScreen IP 0.45 μm Clear Non-sterile plates were from Millipore; guanidine hydrochloride, [tris-(2-carboxyethyl)-phosphine], iodoacetamide, polyvinylpyrrilodone 360 and ammonium bicarbonate were from Sigma.

Protein digestion. In-gel digestion of candidate proteins was performed according to the established protocol (7). Modified porcine trypsin from Promega was used at a final concentration of 12.5 ng/μl. In few cases, polypeptide components of protein complexes were not separated on the gel but directly digested with trypsin utilizing a 98-well PVDF plate format that we have adapted from Papac et al. (8). Briefly, protein was captured onto PVDF membrane of a MultiScreen IP 0.45 μm Clear Non-sterile plate, thoroughly washed, reduced and alkylated with iodoacetamide. Membrane was then blocked with polyvinylpyrrolidone 360, trypsin was added and digestion proceeded at 37° C. for 4 hr. Mixtures of proteolytic peptides were desalted using C18 ziptips, peptides were eluted with 50% AcCN/0.1% TFA.

Sample preparation for MS. For peptide mass fingerprinting (PMF) (9-13) and MS/MS analyses, desalted mixtures of proteolytic peptides were mixed with matrix solution (α-cyano-4-hydroxycinnamic acid 5 mg/ml in 50% ACN/0.1% TFA/10 mM dibasic ammonium phosphate) at a 1:1 ratio directly on a stainless steel target. For MALDI LC MS/MS analysis, samples were separated off-line, as reported previously (4), with the modifications outlined below. The Ultimate 3000 HPLC (Dionex Corporation, Sunnyvale, Calif., USA) that was custom plumbed to accommodate a dual parallel column arrangement was employed. Tryptic digests were separated on monolithic columns (200 μm I.D., 5 cm length, LC Packings, Dionex Corporation, Sunnyvale, Calif., USA) that alternated between a separation and clean up/re-equilibration stage. Following a 5 min isocratic step at 0% B, a linear gradient of 0-70% B in 14 min at a flow rate of 2.5 μl/min was used (A: 0.05% TFA; B: 95% AcCN/0.05% TFA). A SunCollect spotter (SunChrom, Friedrichsdorf, Germany) was used to collect eluate at a rate of one fraction (spot) per five seconds; collection started at 9 min and ended at 19.8 min, counting from the point of injection (129 spots total). Matrix was delivered at a 2.5 μl/min rate and mixed with the column eluate right before spotting onto the MALDI target. MALDI TOF MS and MS/MS Applied Biosystems 4800 Proteomics Analyzer (AB 4800) mass spectrometer (Applied Biosystems, Foster City, Calif., USA/MDS Sciex, Concord, ON, Canada) equipped with TOF/TOF™ ion optics and a 200 Hz NdYag laser (14) and controlled by 4000 Series Explorer Software V3.5.28193 was utilized. MS settings were: m/z range=800-6000 Da; total shots per spectrum=800-1500; single shot protection on (signal 12 intensity range=0-95000); fixed laser intensity=3800-4500. MS/MS data were generated using collision-induced dissociation (CID). MS/MS settings were: m/z range=[60-(10% below the precursor m/z)]; resolution of precursor ion selector=400 FWHM; metastable suppressor: on; total shots per spectrum=1500-4000 with stop conditions (1500 shots in maximum collected for spectra containing>6 peaks with S/N>80); fixed laser intensity=4700-5500; the collision cell was floated at 1 kV; no collision gas was used. AB 4800 MS mode was externally calibrated using Plate Model and Default MS Calibration Update software and employed a combination of six peptide standards (des-Arg₁-bradykinin, angiotensin I, Glut-fibrinopeptide B and three ACTH clips: 1-17, 18-39 and 7-38) with the requirement of at least four standards passing the criteria of S/N of 300, mass tolerance of 0.5 Da, and maximum outlier error of 25 ppm. Default calibration of AB 4800 MS/MS data was based on minimum five matched fragment ions of angiotensin I detected with a minimum S/N of 120, mass tolerance of 2 Da and maximum outlier error of 20 ppm. Automated acquisition of MS and MS/MS data in the batch mode employed an interpretation method with the following settings: number of shots per spot=12; minimum S/N filter=50-80; minimum chromatogram peak width=1 fraction; resolution of precursor exclusion window=200 FWHM; trypsin autolysis peaks were excluded.

MS and MS/MS data analysis. PMF: Mass spectra were processed (baseline adjustment, noise filtering and monoisotopic peak filtering) using Data Explorer Software (Applied Biosystems, Foster City, Calif., USA/MDS Sciex, Concord, ON, Canada) to produce a list of monoisotopic molecular ion masses. Monoisotopic mass peak lists were submitted to the Aldente search engine (15, 16) for protein identification. A combination of two taxa; Desulfovibrio vulgaris subsp. vulgaris str. Hildenborough (DvH) and mammalia (taxon 40674) within UniProtKB/Swiss-Prot (Release 54.8 of 5 Feb. 2008) and UniProtKB/TrEMBL (Release 37.8 of 5 Feb. 2008) were searched using the following parameters: enzyme trypsin: one missed cleavage; fixed modification on Cys: carbamidomethyl (1 allowable; scoring factor 0.9), variable modification on Met: methionine sulfoxide (2 allowable; scoring factor 0.9); thresholds: shift=0.2, slope=200, error=25, minimum hits=4); mass range: 0-250,000 for all polypeptides but DVU101 for which mass range of 0-350,000 was used. Polypeptide identification was considered to be confident when its score was higher than a threshold value which was equal to a score generated by searching a random database, using pValue of 0.05 as a cutoff point; pValue was the probability of finding, for a given spectrum, a protein with the same score in a random protein database. Identities of selected polypeptides that demonstrated relatively low (DVU0460) or below-threshold scores (DVU3242) were confirmed by MS/MS.

MS/MS data were manually matched to the expected sequences. In accordance with the guidelines for publication of proteomics data (17), detailed information on MS-evidence leading to polypeptide identification is provided in Table 51 and Figures S20 to S26, as is indicated below, including PMF data on PMF-only identifications and MS/MS data on identifications based upon single peptides (“one hit wonders”).

LC MALDI MS/MS: Data analysis was performed using ProteinPilot software 13 (Version 2.0, Revision 50861, Applied Biosystems, Foster City, Calif., USA/MDS Sciex, Concord, ON, Canada) with Paragon search engine (18). The custom database that contained all DvH polypeptides and a selection of common contaminants, the latter from Applied Biosystems, was interrogated. The following parameters for ProteinPilot search were utilized: Sample Type: protein identification; Cys alkylation: iodoacetamide; ID Focus: biological modifications and amino acid substitutions; Species: none; Search Effort: thorough; Detection Protein Threshold: 1.3 (95%). Hits were considered to be of high confidence if at least one of at least two distinct peptides had a score of 2 (99% confidence). Polypeptides identified on the basis of less stringent criteria are also reported; their diagnostic MS/MS spectra are not shown.

Electron microscopy. Aliquots of the purified complexes were examined by singleparticle electron microscopy (EM) (29) of negatively stained samples. Uranyl acetate was used as the negative stain in the majority of cases, but ammonium molybdate was tried as a second choice when the results obtained with uranyl acetate were not acceptable. Particles were selected from areas of relatively thick stain in order to minimize the risk of flattening of particles, and images were recorded on film, using a JEOL 4000 microscope operated at 400 keV. Initial models of particle structures were obtained by the random conical tilt (RCT) method (30) whenever either low-pass filtered density maps of 13 homologous structures (e.g. the 70 S ribosome) or intuitive models were not an option. Further details are provided in Han et al, Proc Natl Acad Sci USA. 2009 Sep. 29; 106(39): 16580-16585, including polypeptide sequences, identifying spectra, representative micrographs, details of the reconstruction and refinement strategies, evaluation of the resolution of reconstructions by means of the FSC curve, and validation of results whenever possible by docking either known structures or homology models.

The above examples are provided to illustrate the invention but not to limit its scope. Other variants of the invention will be readily apparent to one of ordinary skill in the art and are encompassed by the appended claims. All references, publications, databases, and patents cited herein are hereby incorporated by reference for all purposes.

TABLE 3
Non-Ribosomal Proteins Detected by Tagless Strategy

| | | | | Poly- | |
|---|---|---|---|---|---|
| Poly- | | | | pep- | |
| pep- | | | | tide | ID |
| tide | | | Uniprot | code | cate- |
| ID# | Polypeptide name | Entry name | accesion# | name | gory^{a} |
| 1 | Pyruvate dehydrogenase E1 component | ODP1_ECOLI | P0AFG8 | AceE | [ID2+] |
| 2 | Dihydrolipoamide acetyltransferase | ODP2_ECOLI | P06959 | AceF | [ID2+] |
| | component of pyruvate dehydrogenase | | | | |
| | complex | | | | |
| 3 | Acyl carrier protein | ACP_ECOLI | P0A6A8 | AcpP | [ID1] |
| 4 | Aldehyde-alcohol dehydrogenase | ADHE_ECOLI | P0A9Q7 | AdhE | [ID1] |
| 5 | Aerobic respiration control protein | ARCA_ECOL6 | P0A9Q2 | ArcA | [ID2+] |
| | arcA | | | | |
| 6 | ATP synthase subunit alpha | ATPA_ECOLI | P0ABB0 | AtpA | [ID2+] |
| 7 | ATP synthase subunit beta | ATPB_ECOLI | P0ABB4 | AtpD | [ID2+] |
| 8 | Bacterioferritin | BFR_ECOLI | P0ABD3 | Bfr | [ID2+] |
| 9 | Lysine decarboxylase, inducible | LDCI_ECOLI | P0A9H3 | CadA | [ID2+] |
| 10 | carbon storage regulator | CSRA_ECOLI | P69913 | CsrA | [ID1] |
| 11 | Sulfite reductase [NADPH] | CYSI_ECOLI | P17846 | CysI | [ID2+] |
| | hemoprotein beta-component | | | | |
| 12 | Sulfite reductase [NADPH] | CYSJ_ECOLI | P38038 | CysJ | [ID2+] |
| | flavoprotein alpha-component | | | | |
| 13 | D-lactate dehydrogenase | DLD_ECOLI | P06149 | Dld | [ID1] |
| | (EC 1.1.1.28) (Respiratory | | | | |
| | D-lactate dehydrogenase) | | | | |
| 14 | DNA polymerase III alpha subunit | DPO3A_ECOLI | P10443 | DnaE | [ID2+] |
| 15 | Chaperone protein dnaJ | DNAJ_ECOLI | P08622 | DnaJ | [ID1] |
| 16 | Chaperone protein dnaK | DNAK_ECOLI | P0A6Y8 | DnaK | [ID2+] |
| 17 | DNA polymerase III subunit tau | DPO3X_ECOLI | P06710 | DnaX | [ID2+] |
| 18 | DNA protection during starvation | DPS_ECOLI | P0ABT3 | Dps | [ID1] |
| | protein | | | | |
| 19 | Probable GTP-binding protein engB | ENGB_ECOLI | P0A6P7 | EngB | [ID2+] |
| 20 | Ferritin-1 | FTNA_ECOLI | P0A998 | FtnA | [ID2+] |
| 21 | Cell division protein ftsY | FTSY_ECOLI | P10121 | FtsY | [ID2+] |
| 22 | Cell division protein ftsZ | FTSZ_ECOLI | P0A9A6 | FtsZ | [ID2+] |
| 23 | Galactitol-specific | PTKA_ECOLI | P69813 | GatA | [ID1] |
| | phosphotransferase | | | | |
| | enzyme IIA component | | | | |
| 24 | Putative tagatose 6-phosphate | GATZ_ECOLI | P37191 | GatZ | [ID1] |
| | kinase gatZ | | | | |
| 25 | Glycine dehydrogenase | GCSP_ECOLI | P33195 | GcvP | [ID1] |
| | [decarboxylating] | | | | |
| 26 | Glycolate oxidase subunit glcD | GLCD_ECOLI | P0AEP9 | GlcD | [ID2+] |
| 27 | glycerol dehydrogenase | GLDA_ECOLI | P0A9S5 | GldA | [ID2+] |
| 28 | Phosphoglucosamine mutase | GLMM_ECOLI | P31120 | GlmM | [ID2+] |
| 29 | glutamine synthetase | GLNA_ECOLI | P0A9C5 | GlnA | [ID2+] |
| 30 | Citrate synthase | CISY_ECOLI | P0ABH7 | GltA | [ID2+] |
| 31 | Glycyl-tRNA synthetase alpha chain | SYGA_ECOLI | P00960 | GlyQ | [ID1] |
| 32 | Glycyl-tRNA synthetase beta chain | SYGB_ECOLI | P00961 | GlyS | [ID2+] |
| 33 | 60 kDa chaperonin | CH60_ECOLI | P0A6F5 | GroL | [ID2+] |
| 34 | Protein grpE | GRPE_ECOLI | P09372 | GrpE | [ID2+] |
| 35 | Inosine-5′-monophosphate | IMDH_ECOLI | P0ADG7 | GuaB | [ID2+] |
| | dehydrogenase | | | | |
| 36 | GMP reductase | GUAC_ECOLI | P60560 | GuaC | [ID2+] |
| 37 | DNA gyrase subunit A | GYRA_ECOLI | P0AES4 | GyrA | [ID2+] |
| 38 | DNA gyrase subunit B | GYRB_ECOLI | P0AES6 | GyrB | [ID2+] |
| 39 | Delta-aminolevulinic acid | HEM2_ECOLI | P0ACB2 | HemB | [ID2+] |
| | dehydratase | | | | |
| 40 | GTP-binding protein hflX | HFLX_ECOLI | P25519 | HflX | [ID2+] |
| 41 | Histidine biosynthesis | HIS7_ECOLI | P06987 | HisB | [ID2+] |
| | bifunctional protein hisB | | | | |
| 42 | Protein transport protein hofQ | HOFQ_ECOLI | P34749 | HofQ | [ID1] |
| | [Precursor] | | | | |
| 43 | DNA polymerase III theta subunit | HOLE_ECOLI | P0ABS9 | HolE | [ID1] |
| 44 | ATP-dependent hsl protease ATP- | HSLU_ECOLI | P0A6H5 | HslU | [ID1] |
| | binding subunit hslU | | | | |
| 45 | Chaperone protein htpG | HTPG_ECOLI | P0A6Z3 | HtpG | [ID2+] |
| 46 | Isoaspartyl dipeptidase | IADA_ECOLI | P39377 | IadA | [ID2+] |
| 47 | Translation initiation factor | IF2_ECOLI | P0A705 | InfB | [ID2+] |
| | IF-2 | | | | |
| 48 | Translation initiation factor | IF3_ECOLI | P0A707 | InfC | [ID2+] |
| | IF-3 | | | | |
| 49 | Peroxidase/catalase HPI | CATA_ECOLI | P13029 | KatG | [ID2+] |
| 50 | Beta-Galactosidase | BGAL_ECOLI | P00722 | LacZ | [ID2+] |
| 51 | Maltoporin [Precursor] | LAMB_ECOLI | P02943 | LamB | [ID1] |
| 52 | GTP-binding protein lepA | LEPA_ECOL6 | P60786 | LepA | [ID1] |
| 53 | Dihydrolipoyl dehydrogenase | DLDH_ECOLI | P0A9P0 | LpdA | [ID2+] |
| 54 | Major outer membrane lipoprotein | LPP_ECOLI | P69776 | Lpp | [ID1] |
| | [Precursor] | | | | |
| 55 | Lysyl-tRNA synthetase, heat | SYK2_ECOLI | P0A8N5 | LysU | [ID2+] |
| | inducible | | | | |
| 56 | NADP-dependent malic enzyme | MAO2_ECOLI | P76558 | MaeB | [ID2+] |
| 57 | S-adenosylmethionine synthetase | METK_ECOLI | P0A817 | MetK | [ID2+] |
| 58 | Chromosome partition protein | MUKB_ECOLI | P22523 | MukB | [ID2+] |
| | mukB | | | | |
| 59 | Chromosome partition protein | MUKE_ECOLI | P22524 | MukE | [ID2+] |
| | mukE | | | | |
| 60 | Chromosome partition protein | MUKF_ECOLI | P60293 | MukF | [ID2+] |
| | mukF | | | | |
| 61 | Transcription elongation protein | NUSA_ECOLI | P0AFF6 | NusA | [ID2+] |
| | nusA | | | | |
| 62 | Peptidoglycan-associated | PAL_ECOLI | P0A912 | PaL | [ID2+] |
| | lipoprotein [Precursor] | | | | |
| 63 | Peptidase B | PEPB_ECOLI | P37095 | PepB | [ID2+] |
| 64 | Phenylalanyl-tRNA synthetase | SYFA_ECOLI | P08312 | PheS | [ID1] |
| | alpha chain | | | | |
| 65 | Polyribonucleotide | PNP_ECOLI | P05055 | Pnp | [ID2+] |
| | nucleotidyltransferase | | | | |
| 66 | Phosphoenolpyruvate carboxylase | CAPP_ECOLI | P00864 | Ppc | [ID2+] |
| 67 | Pyrroline-5-carboxylate reductase | P5CR_ECOLI | P0A9L8 | ProC | [ID2+] |
| 68 | Ribose-phosphate pyrophosphokinase | KPRS_ECOLI | P0A717 | Prs | [ID2+] |
| 69 | phage shock protein | PSPA_ECOLI | P0AFM6 | PspA | [ID2+] |
| 70 | Phosphate acetyltransferase | PTA_ECOLI | P0A9M8 | Pta | [ID2+] |
| 71 | Bifunctional protein putA | PUTA_ECOLI | P09546 | PutA | [ID2+] |
| 72 | Aspartate carbamoyltransferase | PYRB_ECOLI | P0A786 | PyrB | [ID1] |
| | catalytic chain | | | | |
| 73 | Aspartate carbamoyltransferase | PYRI_ECOLI | P0A7F3 | PyrI | [ID1] |
| | regulatory chain | | | | |
| 74 | Protein RecA | RECA_ECOLI | P0A7G6 | RecA | [ID2+] |
| 75 | Transcription termination factor | RHO_ECOLI | P0AG30 | Rho | [ID2+] |
| | rho (ATP-dependent helicase rho) | | | | |
| 76 | Ribosome modulation factor | RMF_EC057 | P0AFW3 | Rmf | [ID1] |
| | (Protein E). | | | | |
| 77 | Ribonuclease I [Precursor] | RNI_ECOLI | P21338 | RnA | [ID1] |
| 78 | Exoribonuclease 2 | RNB_ECOLI | P30850 | Rnb | [ID2+] |
| 79 | DNA-directed RNA polymerase | RPOA_ECOLI | P0A7Z4 | RpoA | [ID2+] |
| | alpha chain | | | | |
| 80 | DNA-directed RNA polymerase | RPOB_ECOLI | P0A8V2 | RpoB | [ID2+] |
| | beta chain | | | | |
| 81 | DNA-directed RNA polymerase | RPOC_ECOLI | P0A8T7 | RpoC | [ID2+] |
| | subunit beta′ | | | | |
| 82 | RNA polymerase sigma-subunit | RPOD_ECOLI | P00579 | RpoD | [ID2+] |
| 83 | DNA-directed RNA polymerase | RPOZ_ECOLI | P0A800 | RpoZ | [ID2+] |
| | omega chain | | | | |
| 84 | Ribosomal small subunit | RSUA_ECOLI | P0AA45 | RsuA | [ID2+] |
| | pseudouridine synthase A | | | | |
| 85 | Preprotein translocase subunit | SECA_ECOLI | P10408 | SecA | [ID2+] |
| | secA | | | | |
| 86 | Outer membrane protein slp | SLP_ECOLI | P37194 | Slp | [ID1] |
| | [Precursor] | | | | |
| 87 | 2-oxoglutarate dehydrogenase | ODO1_ECOLI | P0AFG3 | SucA | [ID2+] |
| | E1 component | | | | |
| 88 | Dihydrolipoamide succinyltransferase | ODO2_ECOLI | P07016 | SucB | [ID2+] |
| | component of 2-oxoglutarate | | | | |
| | dehydrogenase complex | | | | |
| 89 | Thiamine biosynthesis protein thil | THII_ECOLI | P77718 | Thil | [ID2+] |
| 90 | Trigger factor (TF) | TRIG_ECO57 | P0A850 | Tig | [ID2+] |
| 91 | Protein tolB [Precursor | TOLB_ECOLI | P0A855 | TolB | [ID2+] |
| 92 | Elongation factor Tu | EFTU_ECOLI | P0A6N1 | TufA | [ID1] |
| 93 | USG-1 protein | USG_ECOLI | P08390 | Usg | [ID2+] |
| 94 | Protein ybbN | YBBN_ECOLI | P77395 | YbbN | [ID2+] |
| 95 | PhoH-like protein | PHOL_ECOLI | P0A9K3 | YbeZ | [ID2+] |
| 96 | Cellulose synthesis regulatory | YEDQ_ECOLI | P76330 | YedQ | [ID1] |
| | protein | | | | |
| 97 | UPF0265 protein yeeX | YEEX_ECOLI | P0A8M6 | YeeX | [ID2+] |
| 98 | Probable aminotransferase yfbQ | YFBQ_ECOLI | P0A959 | YfbQ | [ID2+] |
| 99 | Hypothetical adenine-specific | YFCB_ECOLI | P39199 | YfcB | [ID2+] |
| | methylase yfcB | | | | |
| 100 | UPF0169 lipoprotein yfiO precursor | YFIO_ECOLI | P0AC02 | YfiO | [ID1] |
| 101 | Hypothetical GTP-binding protein yhbZ | YHBZ_ECOLI | P42641 | YhbZ | [ID2+] |
| 102 | Hypothetical protein yiiU | YIIU_ECOLI | P0AF36 | YiiU | [ID2+] |
| 103 | UPF0307 protein yjgA | YJGA_ECOLI | P0A8X0 | YjgA | [ID1] |

Legend
^{a}Protein identification based on one and two or more peptides for [ID1] and [ID2+] categories,
respectively. MS/MS spectra of polypeptides matched to a single peptide are shown in S [symbol]
^{b}Number of iTRAQ-labeled peptides that contributed to a calculation of average protein relative
ratios, observed in each of the five analyzed four-plexes: “-” marks four-plexes that wer [symbol]
in the course of a repeated LC MALDI TOF MS/MS analysis.
^{c}Unique non-overlapping peptides were used to calculate protein sequence coverage defined as
the ratio between the sum of amino acids encompassed by the confidently matched p [symbol]
and the number of amino acids in a polypeptide sequence. For polypeptides observed in
multiple four-plexes, the best four-plex data are shown.
^{d}GPS software (Applied Biosystems) score: confidence interval (% CI) above 95% signifies that
a given sequence match scored above the Mascot Significance Level calculated for p < [symbol]
i.e., it is considered to be statistically non-random.
^{e}Protein complex data based upon the content of the Encyclopedia of E. coli K-12 Genes and
Metabolism (http://biocyc.org/ECOLI/new-image? object = Protein-Complexes).
^{f}Protein-protein interaction data based upon the study of Butland et al.¹⁵
^{g}Elution profiles were compared using a modified Pearson's algorithm and clusters were defined
employing a threshold of 0.92. Cluster ID “0” means that no partners were found for a [symbol]
Non-Ribosom

[symbol]

| Poly- | | | | Best | | Peptide | | |
|---|---|---|---|---|---|---|---|---|
| pep- | | | Sequence | peptide | | mass | Com- | Inter- |
| tide | #iTRAQ | Polypeptide | coverage | “score” | Best | error | plex | action |
| ID# | peptides^{b} | MW (Da) | (%)^{c} | C.I. %^{d} | Peptide | (ppm) | ID^{e} | ID^{f} |
| 1 | 23/26/29/57/42 | 99,668 | 44.2 | 100 | DYGVGSDVYSVTSFTELAR | | I | |
| 2 | 0/0/9/38/30 | 66,096 | 47.8 | 100 | FNSSLSEDGQR | | I | |
| 3 | 0/0/1/1/1 | 8,640 | 11.5 | 99.996 | IIGEQLGVK | −0.2 | | 3 |
| 4 | 1/0/0/0/0 | 96,127 | 1.1 | 99.851 | DYVEGETAAK | −3.5 | | |
| 5 | 3/0/0/0/0 | 27,292 | 18.1 | 100 | EQANVALMFLTGR | | | |
| 6 | 0/2/0/-- | 55,222 | 1.8 | 95.782 | ASTISNVVR | | D | |
| 7 | 0/4/0/-- | 50,325 | 6.5 | 100 | DVLLFVDNIYR | | D | |
| 8 | 5/0/0/0/0 | 18,495 | 38.0 | 100 | EAIGYADSVHDYVSR | | | |
| 9 | 0/0/6/0/0 | 81,260 | 6.9 | 100 | VIYETQSTHK | | | |
| 10 | 0/0/1/0/0 | 6,856 | 9.8 | 97.677 | EEIYQR | −2.4 | | |
| 11 | 0/0/2/3/0 | 63,998 | 4.2 | 100 | FAGENTIYGSIR | | F | |
| 12 | 0/0/6/5/0 | 66,270 | 8.5 | 100 | VFIEHNDNFR | | F | |
| 13 | 1/0/0/-- | 64,612 | 3.3 | 100 | NQQVFYIGTNQPEVLTEIR | −26.3 | | |
| 14 | 0/2/0/-- | 129,905 | 1.2 | 99.999 | AADQHAK | | E | 6 |
| 15 | 0/1/1/-- | 41,044 | 2.7 | 99.999 | NQGDKEAEAK | 2.7 | C | 2 |
| 16 | 21/9/11/0/0 | 69,115 | 37.0 | 100 | VALQDAGLSVSDIDDVILVGGQTR | | C | 5 |
| 17 | 0/3/0/-- | 71,138 | 4.2 | 100 | ALSNPDLYEGDGELR | | E | 7 |
| 18 | 1/0/0/0/0 | 18,695 | 5.4 | 98.686 | YAIVANDVR | −3.6 | | |
| 19 | 3/0/0/0/0 | 23,561 | 16.2 | 100 | HLPSDTGIEVAFAGR | | | |
| 20 | 0/4/3/0/0 | 19,424 | 21.2 | 100 | LFDYLTDTGNLPR | | | |
| 21 | 0/4/6/-- | 54,513 | 11.7 | 100 | SVMLAAGDTFR | | | |
| 22 | 3/4/2/-- | 40,324 | 14.6 | 100 | GISLLDAFGAANDVLK | | | |
| 23 | 0/1/0/-- | 16,907 | 4.0 | 98.355 | TNLFVR | 2.2 | | |
| 24 | 1/1/0/-- | 47,109 | 1.4 | 99.847 | MVYEAHSTDYQTR | −3.1 | | |
| 25 | 1/0/0/0/0 | 104,376 | 0.9 | 97.62 | AEAAEINLR | −4.2 | K | |
| 26 | 5/0/0/0/0 | 53,812 | 9.6 | 100 | DIEADTPAR | | | |
| 27 | 0/0/2/0/0 | 38,712 | 4.4 | 100 | GLSQGSGVAFDNEK | | | |
| 28 | 4/4/11/3/0 | 47,544 | 25.4 | 100 | IVDAAGR | | | |
| 29 | 0/1/12/0/0 | 51,904 | 24.9 | 100 | AGGVFTDEAIDAYIALR | | | |
| 30 | 2/0/0/0/0 | 48015 | 3.5 | 100 | QLYTGYEK | | | |
| 31 | 1/0/0/-- | 44,716 | 3.3 | 99.953 | AVAEAYYASR | −42.6 | M | |
| 32 | 15/0/0/0/0 | 76,813 | 23.4 | 100 | EAQQLLALENPLPLPAYER | | M | |
| 33 | 0/0/19/38/21 | 57,329 | 45.6 | 100 | ANDAAGDGTTTATVLAQAIITEGLK | | | |
| 34 | 1/3/5/0/0 | 21,798 | 25.4 | 100 | IANLEAQLAEAQTR | | C | 5 |
| 35 | 5/0/0/0/0 | 52,022 | 12.7 | 100 | VGAAVGAGAGNEER | | | |
| 36 | 2/0/0/0/0 | 37,384 | 4.6 | 100 | HVGGVAEYR | | | |
| 37 | 0/0/23/0/0 | 96,964 | 25.4 | 100 | TAEDENVVGLQR | | A | 4 |
| 38 | 5/0/0/0/0 | 89,950 | 5.1 | 100 | AFIEENALK | | A | 4 |
| 39 | 5/0/0/0/0 | 35,625 | 11.7 | 100 | QALDAAGFK | | | |
| 40 | 2/0/0/0/0 | 48,327 | 5.3 | 100 | TGLILDIFAQR | | | |
| 41 | 2/0/0/0/0 | 40,278 | 4.5 | 100 | VEGDTLPSSK | | | |
| 42 | 0/1/0/-- | 44,716 | 1.7 | 99.601 | QEAEQAR | 10.6 | | |
| 43 | 0/1/0/-- | 8,846 | 9.2 | 97.521 | EQPEHLR | 2.1 | E | 6; 17 |
| 44 | 0/0/1/0/0 | 49,594 | 2.0 | 99.974 | FTEVGYVGK | −9.1 | | |
| 45 | 2/0/0/0/0 | 71,423 | 3.7 | 100 | ALSNPDLYEGDGELR | | | |
| 46 | 0/0/0/4/1 | 41,084 | 3.3 | 100 | NVPLFEQALEFAR | | | |
| 47 | 21/5/0/0/0 | 97,350 | 26.1 | 100 | GSSLQQGFQKPAQAVNR | | | |
| 48 | 2/0/0/0/0 | 20,564 | 11.1 | 100 | FRPGTDEGDYQVK | | | |
| 49 | 8/0/0/0/0 | 80,024 | 10.6 | 100 | AVAEVYASSDAHEK | | | |
| 50 | 0/0/9/0/0 | 116,483 | 8.6 | 100 | IDPNAWVER | | | |
| 51 | 0/0/1/1/1 | 49,912 | 3.1 | 100 | GLSQGSGVAFDNEK | 1.8 | | |
| 52 | 0/1/0/-- | 66,570 | 3.0 | 100 | VAEEIEDIVGIDATDAVR | −4.3 | | |
| 53 | 9/5/15/24/16 | 50,688 | 38.0 | 100 | TQVVVLGAGPAGYSAAFR | | I; | |
| | | | | | | | J; | |
| | | | | | | | K | |
| 54 | 1/0/0/0/0 | 8,323 | 15.4 | 99.504 | VDQLSNDVNAMR | −0.6 | B | |
| 55 | 2/0/0/0/0 | 57,827 | 5.0 | 100 | GANEAIDFNDELR | | | |
| 56 | 0/0/6/0/0 | 82,417 | 6.9 | 100 | VLTQEMVK | | | |
| 57 | 0/0/3/5/0 | 41,952 | 7.6 | 100 | QSPDINQGVDR | | | 1; 2 |
| 58 | 0/0/0/35/39 | 170,230 | 25.2 | 100 | SQLADYQQALDVQQTR | | H | 3; 24 |
| 59 | 0/0/0/4/3 | 28,178 | 8.6 | 100 | ITESVFR | | H | 24 |
| 60 | 0/0/0/13/12 | 50,597 | 9.3 | 100 | FTSEQAEGNAIYR | | H | |
| 61 | 0/0/0/12/0 | 54,871 | 16.8 | 100 | ELLEIEGLDEPTVEALR | | G | 9; 10; |
| | | | | | | | | 11; 12; |
| | | | | | | | | 13 |
| 62 | 3/0/2/0/1 | 18,824 | 21.4 | 100 | VTVEGHADER | | B | |
| 63 | 2/0/0/0/0 | 46,180 | 6.8 | 100 | DTINAPAEELGPSQLAQR | | | |
| 64 | 1/0/0/0/0 | 36,832 | 3.1 | 97.864 | EQVQQALNAR | 1.7 | | |
| 65 | 0/0/3/0/0 | 77,101 | 4.5 | 100 | IAATDGEK | | | |
| 66 | 17/0/0/0/0 | 99,063 | 20.7 | 100 | GGAPAHAALLSQPPGSLK | | | |
| 67 | 0/0/2/0/0 | 28,145 | 7.4 | 100 | FAAQAVMGSAK | | | |
| 68 | 1/4/12/6/0 | 34,218 | 43.8 | 100 | LFAGNATPELAQR | | | |
| 69 | 0/0/0/3/0 | 25,493 | 7.7 | 100 | SLDDQFAELK | | | |
| 70 | 20/0/0/0/0 | 77,172 | 31.2 | 100 | VAMLSYSTGTSGAGSDVEK | | | |
| 71 | 13/0/0/2/0 | 143,815 | 10.5 | 100 | GESNILLER | | | |
| 72 | 1/0/0/0/0 | 34,427 | 3.2 | 95.579 | VDEIATDVDK | 3.7 | L | |
| 73 | 1/0/0/0/0 | 17,121 | 6.5 | 99.837 | EFSHNVVLAN | −3.6 | L | |
| 74 | 5/0/1/0/0 | 37,973 | 3.1 | 100 | IVEIYGPESSGK | | | |
| 75 | 7/0/0/2/0 | 47,004 | 18.6 | 100 | GEVVASTFDEPASR | | G | |
| 76 | 1/0/0/0/0 | 6,507 | 16.4 | 99.999 | GYQAGIAGR | −9.4 | | |
| 77 | 0/0/0/0/1 | 29,618 | 2.2 | 99.568 | TFVIDK | −3.4 | | |
| 78 | 5/0/0/0/0 | 72,491 | 7.5 | 100 | ELDAQPTGFLDSR | | | |
| 79 | 0/0/8/14/7 | 36,512 | 27.7 | 100 | LVDIEQVSSTHAK | | G | 9; 14; |
| | | | | | | | | 15; 16; |
| | | | | | | | | 17 |
| 80 | 0/0/5/52/18 | 150,632 | 25.3 | 100 | AVLVAGGVEAEK | | G | 10; 14; |
| | | | | | | | | 18; 19; |
| | | | | | | | | 20 |
| 81 | 0/0/17/57/35 | 155,160 | 28.4 | 100 | LLDLAAPDIIVR | | G | 11; 15; |
| | | | | | | | | 18; 21; |
| | | | | | | | | 22 |
| 82 | 0/0/0/9/2 | 70,263 | 6.2 | 100 | SHATAQEEILK | | G | 12; 16; |
| | | | | | | | | 19; 21; |
| | | | | | | | | 23 |
| 83 | 0/0/2/4/2 | 10,237 | 44.0 | 100 | VTVQDAVEK | | G | 13; 17; |
| | | | | | | | | 20; 22; |
| | | | | | | | | 23 |
| 84 | 4/1/0/0/0 | 25,865 | 20.8 | 100 | LLPEHDVAYDGNPLAQQHGPR | | | |
| 85 | 21/0/0/0/0 | 102,023 | 27.5 | 100 | GEVLENLIPEAFAVVR | | | 1 |
| 86 | 1/0/0/0/0 | 20,964 | 9.6 | 98.813 | SFVAVHNQPGLYVGQQAR | 6.8 | | |
| 87 | 0/0/21/28/0 | 105,062 | 25.2 | 100 | FLSELTAAEGLER | | J | 8 |
| 88 | 0/0/13/18/4 | 44,011 | 39.5 | 100 | ESAPAAAAPAAQPALAAR | | J | 8 |
| 89 | 2/0/0/0/0 | 55,003 | 3.7 | 100 | HYDETLAVVR | | | |
| 90 | 3/0/0/0/0 | 48,193 | 6.5 | 100 | EKDGAVEAEDR | | | |
| 91 | 2/0/0/0/0 | 45,956 | 4.4 | 100 | LPATDGQVK | | B | |
| 92 | 1/0/0/0/0 | 43,314 | 4.1 | 99.332 | GITINTSHVEYDTPTR | 3.4 | | |
| 93 | 0/0/0/0/5 | 36,364 | 13.4 | 100 | AVDALAGQSAK | | | |
| 94 | 0/0/4/7/0 | 31,791 | 16.5 | 100 | KDLTAADGQTR | | | |
| 95 | 7/1/1/0/0 | 39,039 | 22.8 | 100 | AVITGDVTQIDLPR | | | |
| 96 | 0/1/1/-- | 64,283 | 1.2 | 99.841 | AQDVAGR | 7.2 | | |
| 97 | 0/1/2/3/3 | 12,778 | 24.8 | 100 | METTKPSFQDVLEFVR | | | |
| 98 | 0/0/0/2/0 | 45,517 | 2.5 | 100 | RLEEEGNK | | | |
| 99 | 0/0/0/2/0 | 35,002 | 2.9 | 99.99 | IPVAYLTNK | | | |
| 100 | 1/0/0/0/0 | 27,829 | 3.3 | 97.502 | AAFSDFSK | −5.6 | | |
| 101 | 0/0/0/4/2 | 43,286 | 5.1 | 100 | YSQDLAAKPR | | | |
| 102 | 0/0/3/4/3 | 9,635 | 35.8 | 100 | NNSLSQEVQNAQHQR | | | |
| 103 | 1/0/0/0/0 | 21,359 | 4.4 | 99.953 | IPLDADLR | −1.8 | | |

Legend
^{a}Protein identificupplemental FIG. 3.
^{b}Number of iTRAe not analyzed
^{c}Unique non-oveptides (% CI > 95%)
^{d}GPS software (0.05,
^{e}Protein complex
^{f}Protein-protein i [symbol]
^{g}Elution profiles given polypeptide.
Non-Ribosoma

[symbol]

| Polypeptide | Cluster |
|---|---|
| ID# | ID^{g} |
| 1 | 14 |
| 2 | 14 |
| 3 | 0 |
| 4 | 12 |
| 5 | 16 |
| 6 | 10 |
| 7 | 10 |
| 8 | 16 |
| 9 | 0 |
| 10 | 6 |
| 11 | 2 |
| 12 | 0 |
| 13 | 13 |
| 14 | 10 |
| 15 | 0 |
| 16 | 0 |
| 17 | 10 |
| 18 | 3 |
| 19 | 12 |
| 20 | 9 |
| 21 | 17 |
| 22 | 0 |
| 23 | 17 |
| 24 | 3 |
| 25 | 12 |
| 26 | 13 |
| 27 | 5 |
| 28 | 0 |
| 29 | 0 |
| 30 | 1 |
| 31 | 0 |
| 32 | 16 |
| 33 | 4 |
| 34 | 0 |
| 35 | 16 |
| 36 | 1 |
| 37 | 5 |
| 38 | 11 |
| 39 | 12 |
| 40 | 1 |
| 41 | 11 |
| 42 | 0 |
| 43 | 10 |
| 44 | 0 |
| 45 | 12 |
| 46 | 0 |
| 47 | 12 |
| 48 | 11 |
| 49 | 12 |
| 50 | 5 |
| 51 | 0 |
| 52 | 0 |
| 53 | 0 |
| 54 | 13 |
| 55 | 3 |
| 56 | 5 |
| 57 | 4 |
| 58 | 14 |
| 59 | 8 |
| 60 | 8 |
| 61 | 4 |
| 62 | 13 |
| 63 | 12 |
| 64 | 13 |
| 65 | 0 |
| 66 | 12 |
| 67 | 9 |
| 68 | 0 |
| 69 | 15 |
| 70 | 12 |
| 71 | 0 |
| 72 | 11 |
| 73 | 11 |
| 74 | 0 |
| 75 | 1 |
| 76 | 11 |
| 77 | 0 |
| 78 | 1 |
| 79 | 4 |
| 80 | 4 |
| 81 | 4 |
| 82 | 4 |
| 83 | 2 |
| 84 | 3 |
| 85 | 12 |
| 86 | 12 |
| 87 | 7 |
| 88 | 7 |
| 89 | 11 |
| 90 | 1 |
| 91 | 13 |
| 92 | 0 |
| 93 | 14 |
| 94 | 4 |
| 95 | 12 |
| 96 | 0 |
| 97 | 0 |
| 98 | 14 |
| 99 | 15 |
| 100 | 0 |
| 101 | 0 |
| 102 | 6 |
| 103 | 1 |

Legend
^{a}Protein identific [symbol]
^{b}Number of iTRA [symbol]
^{c}Unique non-ove [symbol]
^{d}GPS software ( [symbol]
^{e}Protein complex [symbol]
^{f}Protein-protein i [symbol]
^{g}Elution profiles [symbol] [symbol]
indicates data missing or illegible when filed

TABLE 4
Tagless Strategy-Detection of Reciprocal Protein-Protein Interactions That Were Previously Identified by TAP^{a}

| Inter- | Poly- | Poly- | | | | | | Apex | | |
|---|---|---|---|---|---|---|---|---|---|---|
| action | peptide | peptide | Uniprot | ID | Polypeptide | Sequence | Clustered^{d} | shared^{e} | Co-elution^{f} | All TAP |
| ID | ID# | code name | accesion# | category^{b} | MW (Da) | coverage (%)^{c} | Yes or No | Yes or No | Yes or No | interactions^{a,g} |
| 1 | 57 | MetK | P0A817 | [ID2+] | 41,952 | 7.6 | N | N | N | b9(2_2)_p19 |
| | 85 | SecA | P10408 | [ID2+] | 102,023 | 27.5 | N | N | N | b24(3_1)_p36 |
| 2 | 15 | DnaJ | P08622 | [ID1+] | 41,044 | 2.7 | N | N | N | b36(8_1)_p26 |
| | 57 | MetK | P0A817 | [ID2+] | 41,952 | 7.6 | N | N | N | b9(2_2)_p19 |
| 3 | 3 | AcpP | P0A6A8 | [ID1+] | 8,640 | 11.5 | N | N | Y | b35(6_1)_p1 |
| | 58 | MukB | P22523 | [ID2+] | 170,230 | 25.2 | N | N | Y | b31(1_1)_p5 |
| 4 | 37 | GyrA | P0AES4 | [ID2+] | 96,964 | 25.4 | N | N | N | gyrA_b23(2_1)_p8 |
| | 38 | GyrB | P0AES6 | [ID2+] | 89,950 | 5.1 | N | N | N | gyrB_b14(2_1)_p5 |
| 5 | 16 | DnaK | P0A6Y8 | [ID2+] | 69,115 | 37.0 | N | Y | | dnaK_b31(6_1)_p109 |
| | 34 | GrpE | P09372 | [ID2+] | 21,798 | 25.4 | N | Y | | grpE_b22(1_1)_p6 |
| 6 | 14 | DnaE | P10443 | [ID2+] | 129,905 | 1.2 | Y | Y | | dnaE_b29(3_1)_p2 |
| | 43 | HolE | P0ABS9 | [ID1+] | 8,846 | 9.2 | Y | Y | | holE_b21(4_2)_p2 |
| 7 | 17 | DnaX | P06710 | [ID2+] | 71,138 | 4.2 | Y | Y | | dnaX_b12(2_1)_p3 |
| | 43 | HolE | P0ABS9 | [ID1+] | 8,846 | 9.2 | Y | Y | | holE_b21(4_2)_p2 |
| 8 | 87 | SucA | P0AFG3 | [ID2+] | 105,062 | 25.2 | Y | Y | | sucA_b6(1_1)_p4 |
| | 88 | SucB | P07016 | [ID2+] | 44,011 | 39.5 | Y | Y | | sucB_b5(2_1)_p4 |
| 9 | 61 | NusA | P0AFF6 | [ID2+] | 54,871 | 16.8 | Y | Y | | nusA_b28(6_5)_p3 |
| | 79 | RpoA | P0A7Z4 | [ID2+] | 36,512 | 27.7 | Y | Y | | rpoA_b50(6_5)_p27 |
| 10 | 61 | NusA | P0AFF6 | [ID2+] | 54,871 | 16.8 | Y | Y | | nusA_b28(6_5)_p3 |
| | 80 | RpoB | P0A8V2 | [ID2+] | 150,632 | 25.3 | Y | Y | | rpoB_b46(6_5)_p27 |
| 11 | 61 | NusA | P0AFF6 | [ID2+] | 54,871 | 16.8 | Y | Y | | nusA_b28(6_5)_p3 |
| | 81 | RpoC | P0A8T7 | [ID2+] | 155,160 | 28.4 | Y | Y | | rpoC_b37(7_5)_p31 |
| 12 | 61 | NusA | P0AFF6 | [ID2+] | 54,871 | 16.8 | Y | Y | | nusA_b28(6_5)_p3 |
| | 82 | RpoD | P00579 | [ID2+] | 70,263 | 6.2 | Y | Y | | rpoD_b30(5_5)_p2 |
| 13 | 61 | NusA | P0AFF6 | [ID2+] | 54,871 | 16.8 | N | N | Y | nusA_b28(6_5)_p3 |
| | 83 | RpoZ | P0A800 | [ID2+] | 10,237 | 44.0 | N | N | Y | rpoZ_b23(6_5)_p0 |
| 14 | 79 | RpoA | P0A7Z4 | [ID2+] | 36,512 | 27.7 | Y | Y | | rpoA_b50(6_5)_p27 |
| | 80 | RpoB | P0A8V2 | [ID2+] | 150,632 | 25.3 | Y | Y | | rpoB_b46(6_5)_p27 |
| 15 | 79 | RpoA | P0A7Z4 | [ID2+] | 36,512 | 27.7 | Y | Y | | rpoA_b50(6_5)_p27 |
| | 81 | RpoC | P0A8T7 | [ID2+] | 155,160 | 28.4 | Y | Y | | rpoC_b37(7_5)_p31 |
| 16 | 79 | RpoA | P0A7Z4 | [ID2+] | 36,512 | 27.7 | Y | Y | | rpoA_b50(6_5)_p27 |
| | 82 | RpoD | P00579 | [ID2+] | 70,263 | 6.2 | Y | Y | | rpoD_b30(5_5)_p2 |
| 17 | 79 | RpoA | P0A7Z4 | [ID2+] | 36,512 | 27.7 | N | N | Y | rpoA_b50(6_5)_p27 |
| | 83 | RpoZ | P0A800 | [ID2+] | 10,237 | 44.0 | N | N | Y | rpoZ_b23(6_5)_p0 |
| 18 | 80 | RpoB | P0A8V2 | [ID2+] | 150,632 | 25.3 | Y | Y | | rpoB_b46(6_5)_p27 |
| | 81 | RpoC | P0A8T7 | [ID2+] | 155,160 | 28.4 | Y | Y | | rpoC_b37(7_5)_p31 |
| 19 | 80 | RpoB | P0A8V2 | [ID2+] | 150,632 | 25.3 | Y | Y | | rpoB_b46(6_5)_p27 |
| | 82 | RpoD | P00579 | [ID2+] | 70,263 | 6.2 | Y | Y | | rpoD_b30(5_5)_p2 |
| 20 | 80 | RpoB | P0A8V2 | [ID2+] | 150,632 | 25.3 | N | N | Y | rpoB_b46(6_5)_p27 |
| | 83 | RpoZ | P0A800 | [ID2+] | 10,237 | 44.0 | N | N | Y | rpoZ_b23(6_5)_p0 |
| 21 | 81 | RpoC | P0A8T7 | [ID2+] | 155,160 | 28.4 | Y | Y | | rpoC_b37(7_5)_p31 |
| | 82 | RpoD | P00579 | [ID2+] | 70,263 | 6.2 | Y | Y | | rpoD_b30(5_5)_p2 |
| 22 | 81 | RpoC | P0A8T7 | [ID2+] | 155,160 | 28.4 | N | N | Y | rpoC_b37(7_5)_p31 |
| | 83 | RpoZ | P0A800 | [ID2+] | 10,237 | 44.0 | N | N | Y | rpoZ_b23(6_5)_p0 |
| 23 | 82 | RpoD | P00579 | [ID2+] | 70,263 | 6.2 | N | N | Y | rpoD_b30(5_5)_p2 |
| | 83 | RpoZ | P0A800 | [ID2+] | 10,237 | 44.0 | N | N | Y | rpoZ_b23(6_5)_p0 |
| 24 | 58 | MukB | P22523 | [ID2+] | 170,230 | 25.2 | N | Y | | mukB_b31(2_1)_p5 |
| | 59 | MukE | P22524 | [ID2+] | 28,178 | 8.6 | N | Y | | mukE_b3(1_1)_p0 |

Legend
^{a}Protein-protein interaction data based upon the study of Butland et al.¹⁵
^{b}Protein identification based on one and two or more peptides for [ID1] and [ID2+] categories, respectively. MS/MS spectra of polypeptides matched to a single peptide are shown in Supplemental FIG. 3.
^{c}Unique non-overlapping peptides were used to calculate protein sequence coverage defined as the ratio between the sum of amino acids encompassed by the confidently matched peptides (% CI > 95%) and the number of amino acids in a polypeptide sequence. For polypeptides observed in more than one four-plex, the best four-plex data are shown.
^{d}Elution profiles were compared using modified Pearson's algorithm and clusters were defined employing a threshold of 0.92. Cluster ID “0” means that no partners were found for a given polypeptide.
^{e}Polypetides shared at least one apex of elution.
^{f}Polypeptides eluted in the same fractions.
^{g}A summary of all TAP-derived protein-protein interactions. The following format was used: bN(R_D)_pM, where, b = bait; N = number of interactions reported for the polypeptide acting as a bait; R = number of reciprocal interactions reported for the bait; D = number or reciprocal interactions with partners detected in our study; p = prey; M = number of interactions detected for the polypeptide as a prey only.

TABLE 5
Elution Profile Clustering of Non-Ribosomal Proteins Detected by Tagless Strategy

| Cluster | Polypeptide | Uniprot | Polypeptide | | Polypeptide | Sequence | | Interaction | | | |
|---|---|---|---|---|---|---|---|---|---|---|---|
| ID^{a} | ID# | accesion# | code name | ID category^{b} | MW (Da) | coverage (%)^{c} | Complex ID^{d} | ID^{e} | E7^{f} | E13 | F4 |
| 1 | 30 | P0ABH7 | GltA | [ID2+] | 48015 | 3.5 | | | 1.00 | 0.38 | 0.24 |
| 1 | 36 | P60560 | GuaC | [ID2+] | 37,384 | 4.6 | | | 1.00 | 0.67 | 0.66 |
| 1 | 40 | P25519 | HflX | [ID2+] | 48,327 | 5.3 | | | 1.00 | 0.58 | 0.35 |
| 1 | 75 | P0AG30 | Rho | [ID2+] | 47,004 | 18.6 | G | | 1.00 | 0.62 | 0.56 |
| 1 | 78 | P30850 | Rnb | [ID2+] | 72,491 | 7.5 | | | 1.00 | 0.18 | 0.17 |
| 1 | 90 | P0A850 | Tig | [ID2+] | 48,193 | 6.5 | | | 1.00 | 0.71 | 0.69 |
| 1 | 103 | P0A8X0 | YjgA | [ID1] | 21,359 | 4.4 | | | 1.00 | 0.10 | 0.09 |
| 2 | 11 | P17846 | CysI | [ID2+] | 63,998 | 4.2 | F | | | | |
| 2 | 83 | P0A800 | RpoZ | [ID2+] | 10,237 | 44.0 | G | 13; 17; 20; 22; 23 | | | |
| 3 | 18 | P0ABT3 | Dps | [ID1] | 18,695 | 5.4 | | | 1.00 | 0.67 | 0.84 |
| 3 | 24 | P37191 | GatZ | [ID1] | 47,109 | 1.4 | | | 0.54 | 0.44 | 0.60 |
| 3 | 55 | P0A8N5 | LysU | [ID2+] | 57,827 | 5.0 | | | 0.59 | 0.44 | 0.56 |
| 3 | 84 | P0AA45 | RsuA | [ID2+] | 25,865 | 20.8 | | | 0.21 | 0.27 | 0.42 |
| 4 | 33 | P0A6F5 | GroL | [ID2+] | 57,329 | 45.6 | | | | | |
| 4 | 57 | P0A817 | MetK | [ID2+] | 41,952 | 7.6 | | 1; 2 | | | |
| 4 | 61 | P0AFF6 | NusA | [ID2+] | 54,871 | 16.8 | G | 9; 10; 11; 12; 13 | | | |
| 4 | 79 | P0A7Z4 | RpoA | [ID2+] | 36,512 | 27.7 | G | 9; 14; 15; 16; 17 | | | |
| 4 | 80 | P0A8V2 | RpoB | [ID2+] | 150,632 | 25.3 | G | 10; 14; 18; 19; 20 | | | |
| 4 | 81 | P0A8T7 | RpoC | [ID2+] | 155,160 | 28.4 | G | 11; 15; 18; 21; 22 | | | |
| 4 | 82 | P00579 | RpoD | [ID2+] | 70,263 | 6.2 | G | 12; 16; 19; 21; 23 | | | |
| 4 | 94 | P77395 | YbbN | [ID2+] | 31,791 | 16.5 | | | | | |
| 5 | 27 | P0A9S5 | GldA | [ID2+] | 38,712 | 4.4 | | | | | |
| 5 | 37 | P0AES4 | GyrA | [ID2+] | 96,964 | 25.4 | A | 4 | | | |
| 5 | 50 | P00722 | LacZ | [ID2+] | 116,483 | 8.6 | | | | | |
| 5 | 56 | P76558 | MaeB | [ID2+] | 82,417 | 6.9 | | | | | |
| 6 | 10 | P69913 | CsrA | [ID1] | 6,856 | 9.8 | | | | | |
| 6 | 102 | P0AF36 | YiiU | [ID2+] | 9,635 | 35.8 | | | | | |
| 7 | 87 | P0AFG3 | SucA | [ID2+] | 105,062 | 25.2 | J | 8 | | | |
| 7 | 88 | P07016 | SucB | [ID2+] | 44,011 | 39.5 | J | 8 | | | |
| 8 | 59 | P22524 | MukE | [ID2+] | 28,178 | 8.6 | H | 24 | | | |
| 8 | 60 | P60293 | MukF | [ID2+] | 50,597 | 9.3 | H | | | | |
| 9 | 20 | P0A998 | FtnA | [ID2+] | 19,424 | 21.2 | | | | | |
| 9 | 67 | P0A9L8 | ProC | [ID2+] | 28,145 | 7.4 | | | | | |
| 10 | 6 | P0ABB0 | AtpA | [ID2+] | 55,222 | 1.8 | D | | | | |
| 10 | 7 | P0ABB4 | AtpD | [ID2+] | 50,325 | 6.5 | D | | | | |
| 10 | 14 | P10443 | DnaE | [ID2+] | 129,905 | 1.2 | E | 6 | | | |
| 10 | 17 | P06710 | DnaX | [ID2+] | 71,138 | 4.2 | E | 7 | | | |
| 10 | 43 | P0ABS9 | HolE | [ID1] | 8,846 | 9.2 | E | 6; I7 | | | |
| 11 | 38 | P0AES6 | GyrB | [ID2+] | 89,950 | 5.1 | A | 4 | 1.00 | 0.20 | 0.26 |
| 11 | 41 | P06987 | HisB | [ID2+] | 40,278 | 4.5 | | | 1.00 | 0.52 | 0.81 |
| 11 | 48 | P0A707 | InfC | [ID2+] | 20,564 | 11.1 | | | 0.79 | 0.71 | 1.00 |
| 11 | 72 | P0A786 | PyrB | [ID1] | 34,427 | 3.2 | L | | 1.00 | 0.40 | 0.64 |
| 11 | 73 | P0A7F3 | PyrI | [ID1] | 17,121 | 6.5 | L | | 1.00 | 0.57 | 0.79 |
| 11 | 76 | P0AFW3 | Rmf | [ID1] | 6,507 | 16.4 | | | 1.00 | 0.71 | 0.95 |
| 11 | 89 | P77718 | ThiI | [ID2+] | 55,003 | 3.7 | | | 1.00 | 0.20 | 0.30 |
| 12 | 4 | P0A9Q7 | AdhE | [ID1] | 96,127 | 1.1 | | | 0.98 | 0.62 | 1.00 |
| 12 | 19 | P0A6P7 | EngB | [ID2+] | 23,561 | 16.2 | | | 0.18 | 0.12 | 1.00 |
| 12 | 25 | P33195 | GcvP | [ID1] | 104,376 | 0.9 | K | | 0.35 | 0.37 | 1.00 |
| 12 | 39 | P0ACB2 | HemB | [ID2+] | 35,625 | 11.7 | | | 0.29 | 0.27 | 1.00 |
| 12 | 45 | P0A6Z3 | HtpG | [ID2+] | 71,423 | 3.7 | | | 0.18 | 0.25 | 1.00 |
| 12 | 47 | P0A705 | InfB | [ID2+] | 97,350 | 26.1 | | | 0.06 | 0.04 | 1.00 |
| 12 | 49 | P13029 | KatG | [ID2+] | 80,024 | 10.6 | | | 0.18 | 0.15 | 1.00 |
| 12 | 63 | P37095 | PepB | [ID2+] | 46,180 | 6.8 | | | 0.24 | 0.38 | 1.00 |
| 12 | 66 | P00864 | Ppc | [ID2+] | 99,063 | 20.7 | | | 0.15 | 0.11 | 1.00 |
| 12 | 70 | P0A9M8 | Pta | [ID2+] | 77,172 | 31.2 | | | 0.07 | 0.06 | 1.00 |
| 12 | 85 | P10408 | SecA | [ID2+] | 102,023 | 27.5 | | 1 | 0.20 | 0.42 | 1.00 |
| 12 | 86 | P37194 | Slp | [ID1] | 20,964 | 9.6 | | | 0.16 | 0.17 | 1.00 |
| 12 | 95 | P0A9K3 | YbeZ | [ID2+] | 39,039 | 22.8 | | | 0.20 | 0.15 | 1.00 |
| 13 | 13 | P06149 | Dld | [ID1] | 64,612 | 3.3 | | | 0.39 | 1.00 | 1.00 |
| 13 | 26 | P0AEP9 | GlcD | [ID2+] | 53,812 | 9.6 | | | 0.35 | 0.89 | 1.00 |
| 13 | 54 | P69776 | Lpp | [ID1] | 8,323 | 15.4 | B | | 0.98 | 0.89 | 1.00 |
| 13 | 62 | P0A912 | PaL | [ID2+] | 18,824 | 21.4 | B | | 0.96 | 0.90 | 1.00 |
| 13 | 64 | P08312 | PheS | [ID1] | 36,832 | 3.1 | | | 1.00 | 0.49 | 0.55 |
| 13 | 91 | P0A855 | TolB | [ID2+] | 45,956 | 4.4 | B | | 0.95 | 0.99 | 1.00 |
| 14 | 1 | P0AFG8 | AceE | [ID2+] | 99,668 | 44.2 | I | | 0.04 | 0.01 | 0.01 |
| 14 | 2 | P06959 | AceF | [ID2+] | 66,096 | 47.8 | I | | | | |
| 14 | 58 | P22523 | MukB | [ID2+] | 170,230 | 25.2 | H | 3; 24 | | | |
| 14 | 93 | P08390 | Usg | [ID2+] | 36,364 | 13.4 | | | | | |
| 14 | 98 | P0A959 | YfbQ | [ID2+] | 45,517 | 2.5 | | | | | |
| 15 | 69 | P0AFM6 | PspA | [ID2+] | 25,493 | 7.7 | | | | | |
| 15 | 99 | P39199 | YfcB | [ID2+] | 35,002 | 2.9 | | | | | |
| 16 | 5 | P0A9Q2 | ArcA | [ID2+] | 27,292 | 18.1 | | | 0.22 | 1.00 | 0.36 |
| 16 | 8 | P0ABD3 | Bfr | [ID2+] | 18,495 | 38.0 | | | 0.06 | 1.00 | 0.25 |
| 16 | 32 | P00961 | GlyS | [ID2+] | 76,813 | 23.4 | M | | 0.68 | 1.00 | 0.77 |
| 16 | 35 | P0ADG7 | GuaB | [ID2+] | 52,022 | 12.7 | | | 0.20 | 1.00 | 0.33 |
| 17 | 21 | P10121 | FtsY | [ID2+] | 54,513 | 11.7 | | | | | |
| 17 | 23 | P69813 | GatA | [ID1] | 16,907 | 4.0 | | | | | |
| 0 | 3 | P0A6A8 | AcpP | [ID1] | 8,640 | 11.5 | | 3 | | | |
| 0 | 9 | P0A9H3 | CadA | [ID2+] | 81,260 | 6.9 | | | | | |
| 0 | 12 | P38038 | CysJ | [ID2+] | 66,270 | 8.5 | F | | | | |
| 0 | 15 | P08622 | DnaJ | [ID1] | 41,044 | 2.7 | C | 2 | | | |
| 0 | 16 | P0A6Y8 | DnaK | [ID2+] | 69,115 | 37.0 | C | 5 | 0.42 | 1.00 | 0.67 |
| 0 | 22 | P0A9A6 | FtsZ | [ID2+] | 40,324 | 14.6 | | | 0.79 | 0.88 | 1.00 |
| 0 | 28 | P31120 | GlmM | [ID2+] | 47,544 | 25.4 | | | 0.12 | 0.25 | 0.34 |
| 0 | 29 | P0A9C5 | GlnA | [ID2+] | 51,904 | 24.9 | | | | | |
| 0 | 31 | P00960 | GlyQ | [ID1] | 44,716 | 3.3 | M | | 0.60 | 0.60 | 1.00 |
| 0 | 34 | P09372 | GrpE | [ID2+] | 21,798 | 25.4 | C | 5 | 0.12 | 0.20 | 0.20 |
| 0 | 42 | P34749 | HofQ | [ID1] | 44,716 | 1.7 | | | | | |
| 0 | 44 | P0A6H5 | HslU | [ID1] | 49,594 | 2.0 | | | | | |
| 0 | 46 | P39377 | IadA | [ID2+] | 41,084 | 3.3 | | | | | |
| 0 | 51 | P02943 | LamB | [ID1] | 49,912 | 3.1 | | | | | |
| 0 | 52 | P60786 | LepA | [ID1] | 66,570 | 3.0 | | | | | |
| 0 | 53 | P0A9P0 | LpdA | [ID2+] | 50,688 | 38.0 | I; J; K | | 0.07 | 0.07 | 0.06 |
| 0 | 65 | P05055 | Pnp | [ID2+] | 77,101 | 4.5 | | | | | |
| 0 | 68 | P0A717 | Prs | [ID2+] | 34,218 | 43.8 | | | 0.21 | 0.10 | 0.07 |
| 0 | 71 | P09546 | PutA | [ID2+] | 143,815 | 10.5 | | | 1.00 | 0.22 | 0.22 |
| 0 | 74 | P0A7G6 | RecA | [ID2+] | 37,973 | 3.1 | | | 0.70 | 0.77 | 1.00 |
| 0 | 77 | P21338 | RnA | [ID1] | 29,618 | 2.2 | | | | | |
| 0 | 92 | P0A6N1 | TufA | [ID1] | 43,314 | 4.1 | | | 0.72 | 0.74 | 1.00 |
| 0 | 96 | P76330 | YedQ | [ID1] | 64,283 | 1.2 | | | | | |
| 0 | 97 | P0A8M6 | YeeX | [ID2+] | 12,778 | 24.8 | | | | | |
| 0 | 100 | P0AC02 | YfiO | [ID1] | 27,829 | 3.3 | | | 0.82 | 1.00 | 1.00 |
| 0 | 101 | P42641 | YhbZ | [ID2+] | 43,286 | 5.1 | | | | | |

Legend
^{a}Elution profiles were compared using a modified Pearson's algorithm and clusters were defined employing a threshold of 0.92.
Cluster ID “0” means that no partners were found for a [symbol]
^{b}Protein identification based on one and two or more peptides for [ID1] and [ID2+] categories, respectively. MS/MS spectra of polypeptides matched
to a single peptide are shown in [symbol]
^{c}Unique non-overlapping peptides were used to calculate protein sequence coverage defined as the ratio between the sum of
amino acids encompassed by the confidently matched p [symbol] in a polypeptide sequence. For polypeptides observed in more than one four-plex,
the best four-plex data are shown.
^{d}Protein complex data based upon the content of the Encyclopedia of E. coli K-12 Genes and Metabolism
(http://biocyc.org/ECOLI/new-image?object=Protein-Complexes).
^{e}Protein-protein interaction data based upon the study of Butland et al.¹⁵
^{f}Relative abundance of each polypeptide detected in each of the analyzed fractions. Polypeptide elution profiles are derived from the iTRAQ-based
average relative abundance of eac [symbol] analyzed four-plexes. The results were subsequently equilized among all four-plexes using as reference points
the fractions that were shared between adjacent four-plexes Subsequently, the data were normalized by arbitrarily assigning a value of 1.0 to the fraction
that contained the highest amount of a polypeptide (the highest relative abunda

[symbol]

Elution Profile Clusterin

[symbol]

| Cluster | Polypeptide | | | | | | | | | | | | |
|---|---|---|---|---|---|---|---|---|---|---|---|---|---|
| ID^{a} | ID# | F10* | G1 | G7 | G13* | H4 | H10 | I1* | I7 | I13* | J4* | J10 | K1 |
| 1 | 30 | 0.29 | | | | | | | | | | | |
| 1 | 36 | 0.60 | | | | | | | | | | | |
| 1 | 40 | 0.31 | | | | | | | | | | | |
| 1 | 75 | 0.40 | | | | | | 0.14 | 0.23 | 1.00 | 0.09 | | |
| 1 | 78 | 0.13 | | | | | | | | | | | |
| 1 | 90 | 0.48 | | | | | | | | | | | |
| 1 | 103 | 0.09 | | | | | | | | | | | |
| 2 | 11 | | | | 0.09 | 0.09 | 0.27 | 0.35 | 0.56 | 1.00 | 0.08 | | |
| 2 | 83 | | | | 0.03 | 0.03 | 0.03 | 0.38 | 0.90 | 1.00 | 0.09 | 0.07 | 0.06 |
| 3 | 18 | 0.94 | | | | | | | | | | | |
| 3 | 24 | 1.00 | 0.32 | 0.36 | 0.24 | | | | | | | | |
| 3 | 55 | 1.00 | | | | | | | | | | | |
| 3 | 84 | 1.00 | 0.24 | 0.15 | 0.12 | | | | | | | | |
| 4 | 33 | | | | 0.00 | 0.01 | 0.01 | 0.09 | 1.00 | 0.26 | 0.04 | 0.01 | 0.00 |
| 4 | 57 | | | | 0.14 | 0.13 | 0.16 | 0.42 | 1.00 | 0.68 | 0.11 | | |
| 4 | 61 | | | | | | | 0.06 | 1.00 | 0.19 | 0.03 | | |
| 4 | 79 | | | | 0.06 | 0.11 | 0.09 | 0.40 | 1.00 | 0.56 | 0.09 | 0.06 | 0.05 |
| 4 | 80 | | | | 0.07 | 0.12 | 0.07 | 0.41 | 1.00 | 0.56 | 0.10 | 0.06 | 0.07 |
| 4 | 81 | | | | 0.06 | 0.07 | 0.06 | 0.44 | 1.00 | 0.59 | 0.11 | 0.01 | 0.03 |
| 4 | 82 | | | | | | | 0.08 | 1.00 | 0.57 | 0.07 | 0.06 | 0.06 |
| 4 | 94 | | | | 0.03 | 0.07 | 0.10 | 0.25 | 1.00 | 0.52 | 0.04 | | |
| 5 | 27 | | | | 0.10 | 0.10 | 1.00 | 0.09 | | | | | |
| 5 | 37 | | | | 0.08 | 0.08 | 1.00 | 0.13 | | | | | |
| 5 | 50 | | | | 0.13 | 0.13 | 1.00 | 0.21 | | | | | |
| 5 | 56 | | | | 0.16 | 0.16 | 1.00 | 0.44 | | | | | |
| 6 | 10 | | | | 0.67 | 0.60 | 0.72 | 1.00 | | | | | |
| 6 | 102 | | | | 0.03 | 0.08 | 0.09 | 1.00 | 0.65 | 0.32 | 0.06 | 0.01 | 0.01 |
| 7 | 87 | | | | 0.09 | 0.14 | 1.00 | 0.71 | 0.23 | 0.45 | 0.06 | | |
| 7 | 88 | | | | 0.07 | 0.11 | 1.00 | 0.74 | 0.18 | 0.23 | 0.03 | 0.04 | 0.02 |
| 8 | 59 | | | | | | | 0.22 | 0.72 | 1.00 | 0.71 | 0.64 | 0.18 |
| 8 | 60 | | | | | | | 0.24 | 0.72 | 1.00 | 0.58 | 0.49 | 0.10 |
| 9 | 20 | 0.03 | 0.03 | 0.09 | 1.00 | 0.14 | 0.14 | 0.10 | | | | | |
| 9 | 67 | | | | 1.00 | 0.31 | 0.10 | 0.24 | | | | | |
| 10 | 6 | 0.75 | 1.00 | 0.34 | 0.28 | | | | | | | | |
| 10 | 7 | 0.81 | 1.00 | 0.66 | 0.57 | | | | | | | | |
| 10 | 14 | 0.70 | 1.00 | 0.54 | 0.46 | | | | | | | | |
| 10 | 17 | 0.78 | 1.00 | 0.42 | 0.42 | | | | | | | | |
| 10 | 43 | 0.92 | 1.00 | 0.73 | 0.64 | | | | | | | | |
| 11 | 38 | 0.16 | | | | | | | | | | | |
| 11 | 41 | 0.28 | | | | | | | | | | | |
| 11 | 48 | 0.49 | | | | | | | | | | | |
| 11 | 72 | 0.32 | | | | | | | | | | | |
| 11 | 73 | 0.40 | | | | | | | | | | | |
| 11 | 76 | 0.63 | | | | | | | | | | | |
| 11 | 89 | 0.16 | | | | | | | | | | | |
| 12 | 4 | 0.66 | | | | | | | | | | | |
| 12 | 19 | 0.14 | | | | | | | | | | | |
| 12 | 25 | 0.40 | | | | | | | | | | | |
| 12 | 39 | 0.44 | | | | | | | | | | | |
| 12 | 45 | 0.39 | | | | | | | | | | | |
| 12 | 47 | 0.17 | 0.04 | 0.02 | 0.02 | | | | | | | | |
| 12 | 49 | 0.39 | | | | | | | | | | | |
| 12 | 63 | 0.48 | | | | | | | | | | | |
| 12 | 66 | 0.12 | | | | | | | | | | | |
| 12 | 70 | 0.09 | | | | | | | | | | | |
| 12 | 85 | 0.48 | | | | | | | | | | | |
| 12 | 86 | 0.16 | | | | | | | | | | | |
| 12 | 95 | 0.25 | | | | | | | | | | | |
| 13 | 13 | 0.28 | | | | | | | | | | | |
| 13 | 26 | 0.26 | | | | | | | | | | | |
| 13 | 54 | 0.40 | | | | | | | | | | | |
| 13 | 62 | 0.53 | | | 0.81 | 0.94 | 1.00 | 0.91 | | 1.00 | 0.15 | 0.13 | 0.12 |
| 13 | 64 | 0.25 | | | | | | | | | | | |
| 13 | 91 | 0.75 | | | | | | | | | | | |
| 14 | 1 | 0.06 | 0.14 | 0.03 | 0.04 | 0.04 | 0.06 | 0.05 | 0.11 | 1.00 | 0.05 | 0.02 | 0.02 |
| 14 | 2 | | | | 0.02 | 0.01 | 0.01 | 0.02 | 0.08 | 1.00 | 0.05 | 0.02 | 0.01 |
| 14 | 58 | | | | | | | 0.11 | 0.26 | 1.00 | 0.30 | 0.31 | 0.06 |
| 14 | 93 | | | | | | | | | 1.00 | 0.10 | 0.07 | 0.44 |
| 14 | 98 | | | | | | | 0.07 | 0.21 | 1.00 | 0.18 | | |
| 15 | 69 | | | | | | | 1.00 | 0.95 | 0.85 | 0.14 | | |
| 15 | 99 | | | | | | | 1.00 | 0.98 | 0.98 | 0.20 | | |
| 16 | 5 | 0.18 | | | | | | | | | | | |
| 16 | 8 | 0.11 | | | | | | | | | | | |
| 16 | 32 | 0.99 | | | | | | | | | | | |
| 16 | 35 | 0.21 | | | | | | | | | | | |
| 17 | 21 | 0.45 | 0.39 | 0.48 | 1.00 | 0.35 | 0.37 | 0.39 | | | | | |
| 17 | 23 | 1.00 | 0.54 | 0.57 | 0.71 | | | | | | | | |
| 0 | 3 | | | | 0.10 | 0.15 | 1.00 | 0.28 | 0.37 | 0.38 | 0.11 | 0.09 | 0.02 |
| 0 | 9 | | | | 1.00 | 0.76 | 0.36 | 0.30 | | | | | |
| 0 | 12 | | | | 0.11 | 0.19 | 0.33 | 0.67 | 0.30 | 1.00 | 0.08 | | |
| 0 | 15 | 1.00 | 0.69 | 0.47 | 1.01 | 0.64 | 0.30 | 0.43 | | | | | |
| 0 | 16 | 0.86 | 0.52 | 0.38 | 0.42 | 0.30 | 0.27 | 0.34 | | | | | |
| 0 | 22 | 0.67 | 0.48 | 0.41 | 0.34 | 0.30 | 0.30 | 0.36 | | | | | |
| 0 | 28 | 0.41 | 1.00 | 0.55 | 0.61 | 0.45 | 0.36 | 0.34 | 0.52 | 0.64 | 0.11 | | |
| 0 | 29 | 0.08 | 0.07 | 0.12 | 0.52 | 0.43 | 1.00 | 0.17 | | | | | |
| 0 | 31 | 0.93 | | | | | | | | | | | |
| 0 | 34 | 1.00 | 0.43 | 0.44 | 0.61 | 0.66 | 0.12 | 0.15 | | | | | |
| 0 | 42 | 1.00 | 0.79 | 0.82 | 0.51 | | | | | | | | |
| 0 | 44 | | | | 0.24 | 1.00 | 0.09 | 0.28 | | | | | |
| 0 | 46 | | | | | | | 0.24 | 0.58 | 1.00 | 0.42 | 0.10 | 0.02 |
| 0 | 51 | | | | 0.24 | 0.65 | 0.89 | 1.00 | 0.87 | 0.82 | 0.71 | 0.52 | 0.47 |
| 0 | 52 | 0.27 | 0.23 | 1.00 | 0.31 | | | | | | | | |
| 0 | 53 | 0.05 | 0.04 | 0.02 | 0.04 | 0.06 | 0.16 | 0.07 | 0.07 | 1.00 | 0.04 | 0.02 | 0.01 |
| 0 | 65 | | | | 0.80 | 0.80 | 1.00 | 0.65 | | | | | |
| 0 | 68 | 0.07 | 0.14 | 0.34 | 1.00 | 0.43 | 0.26 | 0.22 | 0.43 | 0.44 | 0.11 | | |
| 0 | 71 | 0.13 | | | | | | 0.23 | 1.00 | 0.46 | 0.08 | | |
| 0 | 74 | 0.81 | | | 0.53 | 0.55 | 0.40 | 1.00 | | | | | |
| 0 | 77 | | | | | | | | | 1.00 | 0.23 | 0.18 | 0.13 |
| 0 | 92 | 0.95 | | | | | | | | | | | |
| 0 | 96 | 1.00 | 0.69 | 0.83 | 0.61 | 0.58 | 0.78 | 0.52 | | | | | |
| 0 | 97 | 0.47 | 0.25 | 0.18 | 0.21 | 0.18 | 0.31 | 0.26 | 0.29 | 1.00 | 0.23 | 0.20 | 0.18 |
| 0 | 100 | 0.95 | | | | | | | | | | | |
| 0 | 101 | | | | | | | 0.15 | 0.33 | 1.00 | 0.85 | 0.08 | 0.09 |

Legend
^{a}Elution profiles were compagiven polypeptide.
^{b}Protein identification based [symbol] upplemental FIG. 3.
^{c}Unique non-overlapping pe [symbol] eptides (% CI > 95%) and the number of amino acids in a polypeptide s [symbol]
^{d}Protein complex data base [symbol]
^{e}Protein-protein interaction d [symbol]
^{f}Relative abundance of each [symbol] h polypeptide within each of the separately analyzed four-ple [symbol] , annotated by an asterisk. Subsequently, the [symbol] nce value) within its contiguous elution chromatogram. [symbol]
indicates data missing or illegible when filed

TABLE 6
Biochemical identity and composition of large macromolecular complexes purified from Desulfovibrio vulgaris
Hildenborough by the tagless strategy. Homologs from other bacteria listed in the rightmost column are members of the same Pfam
families (31) as the D. vulgaris protein.

| | | | | Particle weight | | | |
|---|---|---|---|---|---|---|---|
| | | | Molecular | estimated by SEC | Approximate | | |
| | | | weight of | (weight estimated | number | Stoichiometry | Examples |
| | | | polypeptide | from EM structure | of particles per | (symmetry when | of stoichiometry |
| Gene^{†} | Database annotation | EC number | (kDa) | when known) (kDa) | cell | known)^{#} | in other bacteria^{§} |
| DVU0460 | Predicted phospho-2-dehydro-3- | 2.5.1.54 | 28.4 | 530 | 200 | α₁₆₋₂₀ | α₁₀^{a} |
| | deoxyheptonate aldolase | or | | | | | |
| | | 4.1.2.13 | | | | | |
| DVU0631 | Putative protein | — | 55.7 | 600 | 100 | α₁₀₋₁₄ | — |
| DVU0671 | Putative protein | — | 59.1 | 440 | 700 | α_{8,} | — |
| | | | | (473) | | (D4) | |
| DVU1012 | Hemolysin-type calcium-binding | — | 316.4 | 800 | 1,400 | α₂₋₃ | — |
| | repeat protein | | | | | | |
| DVU1044 | Inosine-5′-monophosphate | 1.1.1.205 | 52.2 | 440 | 800 | α₈ | α₄ |
| | dehydrogenase | | | (418) | | (D4) | |
| DVU1198 | Riboflavin synthase β-subunit | 2.5.1.9 | 16.6 | 600 | 300 | α_{?}β₆₀ | α₃β₆₀; (16) |
| DVU1200 | Riboflavin synthase α-subunit | | 23.6 | (996)^{b} | | (I) | β₅; (32) |
| | | | | | | | β₁₀ (33) |
| DVU1329 | RNA polymerase β-subunit | 2.7.7.6 | 153.2 | 1,100 | 500 | [ββ′α₂ωNusA]₂ | ββ′α₂ω |
| DVU2928 | RNA polymerase (β′-subunit | | 154.8 | (885) | | (C2) | |
| DVU2929 | RNA polymerase α-subunit | | 38.9 | | | | |
| DVU3242 | RNA polymerase ω-subunit | | 8.8 | | | | |
| DVU0510 | NusA | | 47.8 | | | | |
| DVU1378 | Ketol-acid reductoisomerase | 1.1.1.86 | 36.1 | 370 | 600 | α₈₋₁₂ | α₄; |
| | | | | | | | α₁₂ (34) |
| DVU1833 | Phosphoenolpyruvate synthase | 2.7.9.2 | 132.6 | 370 | 1,200 | α₄ | α₂ |
| | | | | (530) | | (apparent D2) | |
| DVU1834 | Pyruvate carboxylase^{c} | 6.4.1.1 | 136.4 | 340 | 800 | [αβ]₂ or [αβ]₄^{d} | [αβ]₄; (35) |
| | | | | | | | [αβ]₄; (36) |
| | | | | | | | [αβ]₁₂ (37) |
| DVU1976 | 60 kDa chaperonin (GroEL) | — | 58.4 | 530 | 700^{e} | α₇ and [α₇]₂ | [α₇]₂ |
| | | | | (409 and 818) | | (C7 and D7) | |
| DVU2349 | Carbohydrate phosphorylase | 2.4.1.1 | 97.4 | 670 | 700 | α₆₋₇ | α₂ |
| | | | | (584) | | (Ring-shaped) | |
| DVU2405 | Alcohol dehydrogenase | 1.1.1.1 | 41.8 | 370 | 12,000 | α₉₋₁₀ | α₂ |
| DVU3025 | Pyruvate:ferredoxin | 1.2.7.1 | 131.5 | 1,000 | 4,000 | [αβδγ]₈, | [αβδγ]₂; |
| | oxidoreductase^{f} | | | (1,052) | | (D4) | [αβδγ]; (38) |
| DVU3319 | Proline dehydrogenase/delta-1- | 1.5.99.8 | 119.0 | 300 | 1100 | α₃ | α₂; |
| | pyrroline-5-carboxylate | and | | | | | α₂ or α₄ (39) |
| | dehydrogenase | 1.5.1.12 | | | | | |

^{†}Entries in bold font indicate protein complexes for which three-dimensional reconstructions were obtained by single-particle electron microscopy (EM) of negatively stained samples.
^{#}Stoichiometry is derived from EM data where we have determined the structure. In other cases, the stochiometry is derived from the SEC size estimation.
^{§}Unless indicated by a specific literature citation, information about subunit stoichiometry was obtained from http://biocyc.org
^{a}E. coli also contains three DAHP synthetases (AroF, AroH and AroG) with stoichiometry α_{2,} α₂ and α₄, respectively. M. tuberculosis AroG has stoichiometry α₅ ((32).) Although Pfam lists Class I aldolases such as DVU0460 in a different family than DAHP synthetases, they are all classified in the same superfamily (Aldolase) in SCOP (40), based on structural evidence of remote homology.
^{b}Contribution of the Riboflavin synthase α-subunit to the particle weight is not included.
^{c}Pyruvate carboxylase is present in some bacteria as a single polypeptide chain and in other bacteria as α and β chains that are homologous to the C- and N- terminal parts, respectively, of the single-chain form of the enzyme. In cases shown here, the α and β chains from other bacteria comprise the same Pfam domains as the single DvH protein. We use αβ to represent the single-chain form.
^{d}EM result indicates either a dimer or tetramer. Size-exclusion chromatography cannot distinguish between these possibilities.
^{e}Particle copy number estimated on the assumption that the protein is present in the cell as a D7 14-mer rather than as the C7 heptamer isolated in our standard buffer conditions.
^{f}Homologs of pyruvate ferredoxin oxidoreductase are sometimes fused and sometimes split into multiple chains. In the case shown here, the α, β, γ, and δ chains from T. maritima comprise the same Pfam domains as the single DvH protein. We use αβδγ to represent the single-chain form.

What is claimed is:

1. A high throughput gel electrophoresis system comprising:
an elution unit comprising a container having
(a) a conduit for liquid movement from the top of the container to the bottom, wherein the container has an opening on top for said conduit and said conduit having an upper region and a lower region, wherein the upper region is of larger diameter and tapers into the lower region, the lower region featuring conducting holes drilled perpendicular to the central axis of the tube to allow liquid communication between solution in the conduit and the container;
(b) a capillary tube with a sleeve inserted into and fitted to the lower region of the conduit, wherein the capillary tube and sleeve extend out through an opening at the bottom of the closed container,
(c) an inlet line to the container connected to a secondary buffer reservoir,
(d) an outlet line from the container for drainage;
(e) extended sidewalls on the top of the container for an upper buffer reservoir;
(f) a metal electrode that can contact with liquid in the container and liquid in the upper reservoir;
(g) a power supply; and
(h) a fraction collector.

2. The system of claim 1 further comprising a linear gel column tube in the upper region of said conduit.

3. The system of claim 2 further comprising a polymerized gel in the gel column tube and buffer solution.

4. The system of claim 1 further comprising:
a first gel segment container having an opening at the bottom of the container and (a) a first gel column tube inserted in the opening, wherein a gel can be formed and polymerized in the first gel column tube, wherein the first gel column tube can fit into the upper region of the conduit when the first gel segment container is placed on top of the elution unit; and (b) a metal electrode that can contact with liquid in the first gel segment container and the metal electrode on the elution unit.

5. The system of claim 4 further comprising:
a second gel segment container having an opening at the bottom of the container and (a) a gel column tube inserted in the opening, wherein a gel can be formed and polymerized in the gel column tube, wherein the first and second gel column tubes fit together end to end such that polymerized gels in the gel column tubes are stacked end to end; and (b) a metal electrode that can contact with liquid in the second gel container and connect to the metal electrode on the first gel segment container or the elution unit.

6. The system of claim 1 wherein the fraction collector is a multi-well plate on a controlled stage.

7. The system of claim 1 wherein the elution unit container and conduit comprising machined plastic or acrylic.

8. The system of claim 1 wherein the capillary tube and sleeve are a narrow-bore glass capillary tube and a PEEK sleeve.

9. The system of claim 1 wherein the metal electrode is platinum.

10. The system of claim 1 wherein the elution unit container having a plurality of opening to accommodate a plurality of conduits with each conduit having a capillary tube with a sleeve inserted into the lower region of the conduit and fitted to the lower region of the conduit, wherein the plurality of capillary tubes and sleeves extend out through a plurality of openings at the bottom of the closed container.

11. The system of claim 10 further comprising:
a first gel segment container having a plurality of openings at the bottom of the container and (a) a plurality of first gel column tubes inserted in the openings, wherein a gel can be formed and polymerized in each first gel column tube, wherein the first gel column tubes can fit into the upper regions of the conduits when the first gel segment container is placed on top of the elution unit; and (b) a metal electrode that can contact with liquid in the first gel segment container and the metal electrode on the elution unit.

12. The system of claim 11 further comprising
a second gel segment container having a plurality of openings at the bottom of the container and (a) a plurality of gel column tubes inserted in the openings, wherein a gel can be formed and polymerized in the gel column tubes, wherein the first and second gel column tubes fit together end to end such that polymerized gels in the gel column tubes are stacked end to end; and (b) a metal electrode that can contact with liquid in the second gel container and connect to the metal electrode on the first gel segment container or the elution unit.

13. A method for biomolecule size separation using electrophoresis comprising (a) providing a polymerized electrophoresis gel loaded with the biomolecules to be separated and purified; (b) performing electrophoresis on said gel to separate the biomolecules; (c) capturing the separated biomolecules as they migrate off the gel.

14. A high throughput method of identifying protein complexes in whole cells from any organism comprising:
passing cell lysate from whole cells through at least two orthogonal separations under conditions that preserve interactions among polypeptide components of protein complexes in the lysate,
collecting polypeptide components in separate elution fractions,
proteolytically digesting each fraction separately to produce a plurality of peptides;
analyzing the peptides in each fraction for peptide identity and abundance of the peptide in the fraction relative to the other fractions, and
identifying co-migrating polypeptides using mathematical analysis based on peptide distribution in the fractions,
wherein co-migrating polypeptides identify protein complexes in the cell.

15. The high throughput method of claim 14, wherein analyzing the peptides in each fraction for protein identity and abundance relative to the other fractions comprises analyzing by mass spectrometry.

16. The high throughput method of claim 14, wherein identifying co-migrating polypeptides comprises clustering.

17. The high throughput method of claim 14, wherein passing cell lysate from whole cells through at least two orthogonal separations comprises passing the cell lysate through a chromatographic separation.

18. The high throughput method of claim 14, further comprising determining a structure of at least one protein complex.

19. The high throughput method of claim 18, wherein determining the structure of at least one protein complex is accomplished by electron microscopy.

20. The high throughput method of claim 14, further comprising storing protein complex information from a plurality of whole cells in an interactive database accessible to a plurality of users, wherein the protein complex information comprises substantially all the protein complexes in the cell.

21. The high throughput method of claim 20, further comprising storing monomer and other single protein information

22. A high throughput method of identifying protein complexes in whole cells from any organism comprising:
providing whole cells,
separating cell lysate from said whole cells under conditions that preserve interactions among polypeptide components of protein complexes in the lysate,
collecting said polypeptide components in separate elution fractions,
proteolytically digesting each fraction separately to produce a plurality of peptides,
analyzing the peptides in each fraction for peptide identity and abundance of the peptide in the fraction relative to the other fractions, and
identifying co-migrating polypeptides using mathematical analysis based on peptide distribution in the fractions, wherein co-migrating polypeptides are protein complexes in the cell.
